SEQUENCE LISTING (42 sequences)

SEQ ID NO: 1 - protein, 1064 aa, Saccharomyces cerevisiae [sequence omitted]

SEQ ID NO: 2 - protein, 1147 aa, Saccharomyces cerevisiae
MPNPNTADYLVNPNFRTSKGGSLSPTPEAFNDTRVAAPATLRMMGKQSGPRNDQQQAPLMPPADIKQGKEQAAQRQNDASRPNGAVELRQFHRRSLGDWEFLETVGAGSMGKVKLVKHRQTKEICVIKIVNRASKAYLHKQHSLPSPKNESEILERQKRLEKEIARDKRTVREASLGQILYHPHICRLFEMCTMSNHFYMLFEYVSGGQLLDYIIQHGSLKEHHARKFARGIASALQYLHANNIVHRDLKIENIMISSSGEIKIIDFGLSNIFDYRKQLHTFCGSLYFAAPELLKAQPYTGPEVDIWSFGIVLYVLVCGKVPFDDENSSILHEKIKKGKVDYPSHLSIEVISLLTRMIVVDPLRRATLKNVVEHPWMNRGYDFKAPSYVPNRVPLTPEMIDSQVLKEMYRLEFIDDIEDTRRSLIRLVTEKEYIQLSQEYWDKLSNAKGLSSSLNNNYLNSTAQQTLIQNHITSNPSQSGYNEPDSNFEDPTLAYHPLLSIYHLVSEMVARKLAKLQRRQALALQAQAQQRQQQQQVALGTKVALNNNSPDIMTKMRSPQKEVVPNPGIFQVPAIGTSGTSNNTNTSNKPPLHVMVPPKLTIPEQAHTSPTSRKSSDIHTELNGVLKSTPVPVSGEYQQRSASPVVGEHQEKNTIGGIFRRISQSGQSQHPTRQQEPLPEREPPTYMSKSNEISIKVPKSHSRTISDYIPSARRYPSYVPNSVDVKQKPAKNTTIAPPIRSVSQKQNSDLPALPQNAELIVQKQRQKLLQENLDKLQINDNDNNNVNAVVDGINNDNSDHYLSVPKGRKLHPSARAKSVGHARRESLKFTRPPIPAALPPSDMTNDNGFLGEANKERYNPVSSNFSTVPEDSTTYSNDTNNRLTSVYSQELTEKQILEEASKAPPGSMPSIDYPKSMFLKGFFSVQTTSSKPLPIVRHNIISVLTRMNIDFKEVKGGFICVQQRPSIETAAVPVITTTGVGLDSGKAMDLQNSLDSQLSSSYHSTASSASRNSSIKRQGSYKRGQNNIPLTPLATNTHQRNSSIPMSPNYGNQSNGTSGELSSMSLDYVQQQDDILTTSRAQNINNVNGQTEQTNTSGIKERPPIKFEIHIVKVRIVGLAGVHFKKVSGNTWLYKELASYILKELNL

SEQ ID NO: 3 - protein, 423 aa, Schizosaccharomyces pombe
MKPNTTNLRNECWDTFSIPKRSQNIKINQSTKHQRSISDFVGTAGPGRQVGNWIIKKTIGAGSMGKVKLVVNILTGEKAALKMIPFTPNNTSQTVRVQREALLGRLLRHPNICRVIDCIRTPACTYILFEYVPGGQLLEYILARGKLDEDLARSFAMQLINALVYLHKNFIVHRDLKIENVLLTQDSRQVKLIDFGLSNFYSKDDLLRTYCGSLYFAAPELLDAKPYIGPEVDVWSLGVVIYVMVCGRVPFDDVSVPMLHSKIKSGKLEFPSYISEDCCSLIAAMLNVNPRKRCSLEQAAKFPWLKKNSFCLYLPIPLTSIPSTPSIRSHVFKPPFNLKVLQLLHEHGLASIPELKHELYMAYIERKTTSLVCLYLLGVESLAPALRIPTALPPVYSRHQRHHSEILGAMDLTEKITAMQCPP

SEQ ID NO: 4 - protein, 891 aa, Schizosaccharomyces pombe
MEYRTNNVPVGNETKSAALNALPKIKISDSPNRHHNLVDAFMQSPSYSTQPKSAVEPLGLSFSPGYISPSSQSPHHGPVRSPSSRKPLPASPSRTRDHSLRVPVSGHSYSADEKPRERRKVIGNYVLGKTIGAGSMGKVKVAHHLKTGEQFAIKIVTRLHPDITKAKAAASAEATKAAQSEKNKEIRTVREAALSTLLRHPYICEARDVYITNSHYYMVFEFVDGGQMLDYIISHGKLKEKQARKFVRQIGSALSYLHQNSVVHRDLKIENILISKTGDIKIIDFGLSNLYRRQSRLRTFCGSLYFAAPELLNAQPYIGPEVDVWSFGIVLYVLVCGKVPFDDQNMSALHAKIKKGTVEYPSYLSSDCKGLLSRMLVTDPLKRATLEEVLNHPWMIRNYEGPPASFAPERSPITLPLDPEIIREMNGFDFGPPEKIVRELTKVISSEAYQSLAKTGFYSGPNSADKKKSFFEFRIRHAAHDIENPILPSLSMNTDIYDAFHPLISIYYLVSERRVYEKGGNWNRIAKTPVSSVPSSPVQPTSYNRTLPPMPEVVAYKGDEESPRVSRNTSLARRKPLPDTESHSPSPSATSSIKKNPSSIFRRFSSRRKQNKSSTSTLQISAPLETSQSPPTPRTKPSHKPPVSYKNKLVTQSAIGRSTSVREGRYAGISSQMDSLNMDSTGPSASNMANAPPSVRNNRVLNPRGASLGHGRMSTSTTNRQKQILNETMGNPVDKNSTSPSKSTDKLDPIKPVFLKGLFSVSTTSTKSTESIQRDLIRVMGMLDIEYKEIKGGYACLYKPQGIRTPTKSTSVHTRRKPSYGSNSTTDSYGSVPDTVPLDDNGESPASNLAFEIYIVKVPILSLRGVSFHRISGNSWQYKTLASRILNELKL

SEQ ID NO: 5 - protein, 1212 aa, Candida albicans
MNNQDPDSQYHNKKVYPPNLPSIPPPPQQPLSGRPATPRMLRSISGTLKSKTELAHSDKGQESNNETKNSNSPHYVPDTHTRQPPPESLKSNIQAPTAVHGNQQKGSLLPPPSIPNPNTMKPAPTPTGVDQPPAKQKPSPAPKQPQPQQQQQQQQQQQFHRKSIGDWNFVKTIGAGSMGKVKLAQHNATHEICAVKIIPRAAKLYQRAHANDPPPQTTQEAAQRHKEFEKEVARDRRTIREGALGRLLYHPFICRLYEMVPMTNHYYMLFEYIEGGQMLDYIVAHGSLKERHARKFARGIASALDYCHRNNVVHRDLKIENIMINEKGDIKIIDFGLSNLYAPKNLLKTYCGSLYFAAPELLSAKPYIGPEVDVWSFGVVLYVLVCGKVPFDDQSVSVLHEKIKKGNVEYPAFLSRECVSLLSRMLVVDPTKRASLYEVCSHPWMNKGYDYKVNNYLPRREPLRLPLDPEIIKTIANFELGTVQGVADELTSILTSVEYQMSCENWYKITETGREYASSQNAQILPDPTGGFHPLVSIYYLVDEMRKRKKAKEEALKAQRRAQVPTIAVPTPKQQQQQQPQPAQPQPQPQPEVSQPLPEPKPVPPEEIINPAVATQAQANMTAPKIVETFSETPQRTLDPSKQSVDEKPSAPGPSIAVPEQAHTTSVPSSFVKTQTSIDEDQLSIPEQQSPRTSTPQTLDPAKVVGGSSGSAISAPNAGSGAGFNSLLRRLSSKKYKGASSPKRSTSPSPNVEGLSPQPTKADPMVRRGVSMKVTAKEKQTNTRPPKSELIKKKPQHGRSSSTSNKMQGFIPVEYLPPLPTIDTNTNTIVSDGAKQQNLTVPSTARHMHPTARAKSVGGGHMRKDSYGRVSHGSQNPLPPLPTSMASQNSQEVVGKDTSEGFFDDVQLDDVGYQEVPQLTESEIIEQYNISKPNSMPSIEHCKTLFLKGFFSVQTTSAKPLPVIRYNIINVLSKLGVKFQEVKGGFVCMHTPSVQPSHSNELDEENKLYGDAFKSKSSDSFEAAEPEGSKTPSRQPSLQLPSHTPTTPSGPKSHKSSNSIGSIGGNVPRRKFSIGNAFNTYRKKNGSQVMMPPNTPATAKVIHGLYDDDDKERNGEDDDDEDDYGYDDSADSLNGYGGGSDMLISSRIEQRAKHQRTVSSSSQKASKSPLKFEIHIVKVPLVGLYGVQFKKILGNTWNYKTLASQILNEMNL

SEQ ID NO: 6 - protein, 800 aa, Saccharomyces cerevisiae
MASVPKRHTYGGNVVTDRDRHSLQRNNEILHPIHKNQRKHATFGPYIIGSTLGEGEFGKVKLGWTKASSSNEVPKQVAIKLIRRDTIKKDADKEIKIYREINALKHLTHPNIIYLEEVLQNSKYIGIVLEFVSGGEFYKYIQRKRRLKESSACRLFAQLISGVNYMHYKGLVHRDLKLENLLLDKHENLVITDFGFVNEFFEDNELMKTSCGSPCYAAPELVVSTKAYEARKADVWSCGVILYAMLAGYLPWDDDHENPTGDDIARLYKYITQTPLKFPEYITPIPRDLLRRILVPNPRRRINLQTIKRHVWLKPHEAFLSIQPNYWDEHLQKERPKPPNKGDVGRHSTYSSSASSYSKSRDRNSLIIESTLEQHRMSPQLATSRPASPTFSTGSKVVLNDTKNDMKESNINGERTSASCRYTRDSKGNGQTQIEQVSARHSSRGNKHTSVAGLVTIPGSPTTARTRNAPSSKLTEHVKDSSQTSFTQEEFHRIGNYHVPRSRPRPTSYYPGLSRNTADNSLADIPVNKLGSNGRLTDAKDPVPLNAIHDTNKATISNNSIMLLSEGPAAKTSPVDYHYAIGDLNHGDKPITEVIDKINKDLTHKAAENGFPRESIDPESTSTILVTKEPTNSTDEDHVESQLENVGHSSNKSDASSDKDSKKIYEKKRFSFMSLYSSLNGSRSTVESRTSKGNAPPVSSRNPSGQSNRSNIKITQQQPRNLSDRVPNPDKKINDNRIRDNAPSYAESENPGRSVRASVMVSTLREENRSELSNEGNNVEAQTSTARKVLNFFKRRSMRV

SEQ ID NO: 7 - protein, 865 aa, Saccharomyces cerevisiae
MSYTNKRHTYYGGFTNDLSDTFQYPQRTDEQRRKHVTFGPYILGSTLGEGEFGKVKLGWPKNFSNSSNSTFDFPKQVAIKLIKRDSISNDYRKEVKIYREINALKHLSHPNIVKLEEVLQNSRYIGIVLEYACGGEFYKYIQKKRRLKEMNACRLFSQLISGVHYIHSKGLVHRDLKLENLLLDKNENLVITDFGFVNEFCSRNELMKTSCGSPCYAAPELVISAEPYEARKADIWSCGVILYAILAGYLPWDDDPNNPEGSDIGRLYNYINSTPLKFPDYILPIPRDLLRRMLVSDPKKRINLKQIKKHEWLKPHSSFLSITPDEWDKLNNTQSVFRLAKPRRRYGSRPQSSCSTSSLGSRSDKRDSLVIDSTLITFPAPPQESQNHIITRPASIASDQRLSPIRRSNRHNRSNSAASVALQAVVNADREYVLSHEQSLSPVQNIRQTTGNMTASLSPPPAISPGDIIIETTPIKRNTISGSSIVPSLEEESSTTMQTSKIQPNNMASSQNHQYNKNKTQNSLQSAKNFYRTSSSSHTKPRPTSYHPGSYTTPPYNSNTLSIYEINEKAKSSASSQTLNQRDTSPFDSTPYLALDTCITSSSSIESSPKLITHGQFSVAKPSVDLQSVSGDLIKYKRDADVVTRIYDEKYKQKRKSLRYSGIFSDISCDTVTEESDELRPPESPLQQHEGQESIDKAKTEDTSEKGSKSSNIAKATAQKHVNNHLERSLNEAESTKKRFSFLSLYSYDTSKSSLYSSMDSKRKPSPPSQRRPKKDDSYQTNSKNHYITASNMQTSHQVSKDLPAPTMVQNKCTLETKKAVRSNRSSIMVSEVNKASVDNKAAQSPEHSTAKRVLGFFKRRSMKI

SEQ ID NO: 8 - protein, 994 aa, Candida albicans
MSTIPSQVEINFNKIHQRSNSSSSTSSYRIPSGNSCIPRTVEMPSLPPTSTHHQHQQMPSSSSHAHIAKKIHREVRFGAYILGSTLGEGEFGKVKLGWRKDGKHPSQVAIKLIKRSTITKDSDSEIKIHREINSLKLLNHPNIVNLVEVMKSGKYIGIVLEYASGGELFDYILQHKYLKENVAKKLFAQLVSGVDYMHAKGLIHRDLKLENLLLDKHRNVIISDFGFVNSYNRDKNDLMKTSCGSPCYAAPELVLSQTAYEGRKVDIWSLGVILYAMLAGYLPFDDDPENEDGSDIIKLYHYICKTPLTFPEYVSPLARDLLRKIIVSDPKKRISIDDIRNHPWLSSHANLLSIRQPEWDKVHSEKQQPIAVEPPQPNKRYSMINERTNSSSLMSPAPRVTHTQPLSSHARSYSSTSISLLYSSPSATPSMANAVTNGEGATTTTTNGSINESNDTLQLSGTPSPKKPSTVSPVRGHQKSASISNSYSSASIALKAVVHEENRLHNHQQSQQYIPRSSTISTIVESPTKANTATETETTDGHKILLPPPSKDAQKLPHAAKKPRPTSYHPSSMSSALIHNNQNPTDVLKMPSPINFPMTQFISTSPPKSNGSLNDSGNFTNCSPKATSRRNSVVTHVHVNGVLSKENLIHSSSSPDNKRNSVLSYLEDKIDTLELTESHSPSKNTFEEIVDAAIATPEINQVPVFDSQTSPNGIGLDIKHKEFDETSLVVEKKSKETVSDSKSEESTKETQQQENVVMYEPIVPVEEYIKKPEEVNSVEAKQPEEVKSEDKSLQGQKSQQQQQQPEKHSADIGKTKVDLKKSASQKKKVKEESIKKQKDVDSKPIERRHTIAARRHHNDENKENKDVKKRNRFSLLSFYSSYNSSNSNVSLATSKVPSNSENNTTVLKPTSMNTTRKVLEPSNETNIMRKETKQTNSNGSTTSKSTTSSSSSSAPAASSSSSSSTSKRASTATKETSAARKVMDFFKRRSVRVG

SEQ ID NO: 9 - protein, 629 aa, Schizosaccharomyces pombe
MNAQPFHNNTSDVQSFQDIISNSYQKPLSLVDSTDRALPDSSLSSLSRSTFQFHKHHLSGNENPQPSSESPYFTNNERLNSSSFPQIHDNQLSPSFNTSYQAIPSSSSNRSRGGPYTPSIRDDSLLALLSFSSNHRLHSMPSQLQPFNNASSYTTPMAPFTASFSNKVSHSAYPTRRLPSQAKKTSAIERVPVNLNFLQSDNLVVQSSPQTNFENFEFPKKIPSKEDLETREVLLLPPQTSKLSNKNLDTKSFTDVNKISQQGFVEISSNSSKVTPNTSLHQSFGIASSSSNNYMQTSSELTSSTEKMNGSHPLQLSNKSLLSIHLMQSKNQGHVSMTGSDKLSSHVQSETENAPVSKPSKPNTLTEDEKPLQSTKLPGNSLTVGELYQEPKSIQLPELSVSRTTYSAQSSSVKNCNERIPSAKALKKQKHLVPENKSKLQYVWQKKESLPYANLTSASNTHFFLSENQNDTSERLTRTLRKSTKNYTFGSYILGRTIGTGEFGKVKLGWPLPKANSTIHRSTPQVVIKIVLSTKQNCQTSRLMREVAILKGLGNNHPHIVKYLDFVKTKHHFGIVLDYVNGGELFDYILARRRLEDSVACRLFAQLISGVAYLHSRGVVHRDPYSESY

SEQ ID NO: 10 - protein, 1142 aa, Saccharomyces cerevisiae
MAINGNSIPAIKDNTIGPWKLGETLGLGSTGKVQLARNGSTGQEAAVKVISKAVFNTGNVSGTSIVGSTTPDALPYGIEREIIIMKLLNHPNVLRLYDVWETNTDLYLVLEYAEKGELFNLLVERGPLPEHEAIRFFRQIIIGVSYCHALGIVHRDLKPENLLLDHKYNIKIADFGMAALETEGKLLETSCGSPHYAAPEIVSGIPYQGFASDVWSCGVILFALLTGRLPFDEEDGNIRTLLLKVQKGEFEMPSDDEISREAQDLIRKILTVDPERRIKTRDILKHPLLQKYPSIRDSKSIRGLPREDTYLTPLSESNSSIDATILQNLVILWHGRDPEGIKEKLREPGANAEKTLYALLYRFKCDTQKELIKQQQVKKRQSISSVSVSPSKKVSTTPQRRRNRESLISVTSSRKKPISFNKFTASSASSSNLTTPGSSKRLSKNFSSKKKLSTIVNQSSPTPASRNKRASVINVEKNQKRASIFSTTKKNKRSSRSIKRMSLIPSMKRESVTTKLMSTYAKLAEDDDWEYIEKETKRTSSNFATLIDEIFEYEKYEQIRKEKEELERKVREAKAREELERRRRKQEEKERARKLLEKEDLKRKQEELKKQIEIDISDLEQELSKHKEEKLDGNIRSISAPMENEEKNINHLEVDIDNILRRRNFSLQTRPVSRLDPGIMFSSPTEEVSPVEPKRTENERLTTEKKILETIRRSKFLGSSFNIDKELKLSKMEYPSIIAPQRLSEERVVSDSNDGYESLILPKDGNGVSQLKDSTATTAPVSDGRLRKISEIRVPQFTRKSRHFSESNKRLSVLSMYSTKESFTNLVDILKNGNLDVNNQQSQRIPTPRSADDSEFLFETVNEEAEYTGNSSNDERLYDVGDSTIKDKSALKLNFADRFNGSNEAKQTDNLHLPILPPLNGDNELRKQNSQEGDQAHPKIKSMIPESGSSSHTEKEEENEEKEEKKPEQHKQEEDQEKREKVVDDMEPPLNKSVQKIREKNAGSQAKDHSKDHLKEHKQDKNTAIGNGSFFRKFSKSSDKTMELYAKISAKQLFNGLEKLLRGWTQYGLKNIKSHPNNLTLTGKLSSDNIFSLRSTLFEVNIYPRGKMSVVQFKKVSGSFKAVKKLVNEVENVLNKEGVLQK

SEQ ID NO: 11 - protein, 1518 aa, Saccharomyces cerevisiae
MTGHVSKTSHVPKGRPSSLAKKAAKRAMAKVNSNPKRASGHLERVVQSVNDATKRLSQPDSTVSVATKSSKRKSRDTVGPWKLGKTLGKGSSGRVRLAKNMETGQLAAIKIVPKKKAFVHCSNNGTVPNSYSSSMVTSNVSSPSIASREHSNHSQTNPYGIEREIVIMKLISHTNVMALFEVWENKSELYLVLEYVDGGELFDYLVSKGKLPEREAIHYFKQIVEGVSYCHSFNICHRDLKPENLLLDKKNRRIKIADFGMAALELPNKLLKTSCGSPHYASPEIVMGRPYHGGPSDVWSCGIVLFALLTGHLPFNDDNIKKLLLKVQSGKYQMPSNLSSEARDLISKILVIDPEKRITTQEILKHPLIKKYDDLPVNKVLRKMRKDNMARGKSNSDLHLLNNVSPSIVTLHSKGEIDESILRSLQILWHGVSRELITAKLLQKPMSEEKLFYSLLLQYKQRHSISLSSSSENKKSATESSVNEPRIEYASKTANNTGLRSENNDVKTLHSLEIHSEDTSTVNQNNAITGVNTEINAPVLAQKSQFSINTLSQPESDKAEAEAVTLPPAIPIFNASSSRIFRNSYTSISSRSRRSLRLSNSRLSLSASTSRETVHDNEMPLPQLPKSPSRYSLSRRAIHASPSTKSIHKSLSRKNIAATVAARRTLQNSASKRSLYSLQSISKRSLNLNDLLVFDDPLPSKKPASENVNKSEPHSLESDSDFEILCDQILFGNALDRILEEEEDNEKERDTQRQRQNDTKSSADTFTISGVSTNKENEGPEYPTKIEKNQFNMSYKPSENMSGLSSFPIFEKENTLSSSYLEEQKPKRAALSDITNSFNKMNKQEGMRIEKKIQREQLQKKNDRPSPLKPIQHQELRVNSLPNDQGKPSLSLDPRRNISQPVNSKVESLLQGLKFKKEPASHWTHERGSLFMSEHVEDEKPVKASDVSIESSYVPLTTVATSSRDPSVLAESSTIQKPMLSLPSSFLNTSMTFKNLSQILADDGDDKHLSVPQNQSRSVAMSHPLRKQSAKISLTPRSNLNANLSVKRNQGSPGSYLSNDLDGISDMTFAMEIPTNTFTAQAIQLMNNDTDNNKINTSPKASSFTKEKVIKSAAYISKEKEPDNSDTNYIPDYTIPNTYDEKAINIFEDAPSDEGSLNTSSSESDSRASVHRKAVSIDTMATTNVLTPATNVRVSLYWNNNSSGIPRETTEEILSKLRLSPENPSNTHMQKRFSSTRGSRDSNALGISQSLQSMFKDLEEDQDGHTSQADILESSMSYSKRRPSEESVNPKQRVTMLFDEEEEESKKVGGGKIKEEHTKLDNKISEESSQLVLPVVEKKENANNTENNYSKIPKPSTIKVTKDTAMESNTQTHTKKPILKSVQNVEVEEAPSSDKKNWFVKLFQNFSSHNNATKASKNHVTNISFDDAHMLTLNEFNKNSIDYQLKNLDHKFGRKVVEYDCKFVKGNFKFKIKITSTPNASSVITVKKRSKHSNTSSNKAFEKFNDDVERVIRNAGRS

SEQ ID NO: 12 - protein, 1037 aa, Saccharomyces cerevisiae
MTVANTETHSAAKPSSTIGPWKLGETLGFGSTGKVQLAQHERTGHRTAVKVISKSIFNNNGNHSNDDSVLPYNIEREIVIMKLLSHPNVLSLYDVWETNNNLYLILEYAEKGELFNLLVDHGPLPEREAINCFRQIIIGISYCHALGIVHRDLKPENLLLDSFYNIKIADFGMAALQTDADLLETSCGSPHYAAPEIVSGLPYEGFASDVWSCGVILFALLTGRLPFDEENGNVRDLLLKVQKGQFEMPNDTEISRDAQDLIGKILVVDPRQRIKIRDILSHPLLKKYQTIKDSKSIKDLPRENTYLYPLADSNNHTSASIDDSILQNLVVLWHGRHADDIVSKLKENGTNKEKILYALLYRFKLDSVRGSNKKNRNKIKKTKKNKRSSTLSSSSSLLLNNRSIQSTPRRRTSKRHSREFSSSRKRSSFLLSSNPTDSSPIPLRSSKRITHINVASANTQATPSGVPNPHKRNSKKRSSKRLSYMPNTKRSSLTSKSLSNFTNLIDDDDWEYIEKDAKRTSSNFATLIDEIFEPEKFELAKREKAELQRKVQEAKRQSVNAQKINEDEFGSEVSDGMKELKKINDKVSSPLINYEFSQQELLQDIDTLLTNRYQLSSYTRPISRLDPGLTPVTETLPNNLKEKTALLQDTEKKIIETIRRSKFLGSLLNVRGGLSPGKSELAPIEESPIVSTTPLIYNDRMEPRRISDVEVPHFTRKSKHFTTANNRRSVLSLYAKDSIKDLNEFLIKEDPDLPPQGSTDNESRSEDPEIAESITDSRNIQYDEDDSKDGDNVNNDNILSDFPQGVGISQEYDMKDKNPNQSPISKSAEPTLVVKLPSLSSFQGKNASGLGLYQREPSKVTLPSLTSNNSSVGENIEDGAEKGTESEKIAASLSDDDLKEDNDKKDNDTVNAPTTVKKPPNSVLLKKFSKGKILELEIHAKIPEKRLYEGLHKLLEGWKQYGLKNLVFNITNMIITGKLVNDSILFLRSTLFEIMVLPNGDGRSLIKFNKKTGSTKTLTKLATEIQIILQKEGVLDK

SEQ ID NO: 13 - protein, 775 aa, Schizosaccharomyces pombe
MSTISEVGPWELGLSLGSGGPNSSRLAKHRETGQLAVVKPIVGWSELTSSQQARIEGELVLLRLIEHPNVLQLIDVISAQEQLFVVVEYMPGGELFDCMLRKGSFTEQDTAKFLWQILCGLEYCHKLHICHRDLKPENLYLDAHGSIKIGEFGMASIQQPGKLLQTSCGSPHYASPEIIMGRSYDGCASDIWSCGIIFFALLTGKLPFDDDNIRSLLLKVCQGQFEMPSNISPQAQHLLYRMLDVDSSTRITMEQIREHPFLSCFVHPNISIPIISAPIQPIDPLIVQHLSLVFRCSDDPMPLYEKLASQSPLVEKTLYTLLSRHLHPPSSAAVDRNRAVVDDLLGTAASNGQQMDEEEIEQAINIPTLAPYPISYAAESVPRPATSASPFLTPVTTSGTFNYSFNATNPQSILQRPATTSSAVPQLPKSVTPGLAYPHDSSMLSSNYRPPSALSPRNFNVSINDPEVQLSRRATSLDMSNDFRMNENDPSIVGNLAASNFPTGMGPPRKRVTSRMSEHTGNRVVSFPRGSAFNPRVTRFNVGNEQFSNNIDNNNYNQPYANATMNNSRRLRTPSGERSMRADLSQSPASYDSLNVPKHRRRQSLFSPSSTKKKLSGSPFQPKRSFLRRLFSSEPSCKCVYASLVASELEHEILEVLRRWQLLGIGIADIIYDSVSASISARIKRQNSLNLKPVRFRISVLAEFFGSQAVFVLESGSSTTFDHLATEFQLIFEDKGFLDNLELSYFQASASRPVSRMSVSSSPFAVFRQRQSVQS

SEQ ID NO: 14 - protein, 593 aa, Schizosaccharomyces pombe
MVKRHKNTIGVWRLGKTLGTGSTSCVRLAKHAKTGDLAAIKIIPIRYASIGMEILMMRLLRHPNILRLYDVWTDHQHMYLALEYVPDGELFHYIRKHGPLSEREAAHYLSQILDAVAHCHRFRFRHRDLKLENILIKVNEQQIKIADFGMATVEPNDSCLENYCGSLHYLAPEIVSHKPYRGAPADVWSCGVILYSLLSNKLPFGGQNTDVIYNKIRHGAYDLPSSISSAAQDLLHRMLDVNPSTRITIPEVFSHPFLMGCTSLSSMDSTTPPTPSLSIDEIDPLVVDCMCVLWKKSSSKKVVRRLQQRDDNDEKYVYKVLSEILRDDMLKKQRFDENKYLSLYDLIHDNNLFTKASISTTSLVKSNVSTNSRKSSNFEDELARRVSSPLSALNQMSQSPIPIRVSSDKDYDSYACHEVVSNPSTLDDDYNYMFVCPPEEYTYSTDNVRTDSLDLQSLPTPTLEQLESVPFNRYGYVRIFPSTTLSSTASGYYTPDSLSTPEPSIDGLTNLDDVQVGGFVQGSGNQNRRPISFPVISNMQPNITNVRSASAPLCSSPVPSRRYSQYATNARYTPRKVSSGSVLRKISSFFRKD

SEQ ID NO: 15 - protein, 1462 aa, Candida albicans
MSTVVNRRSSHQFDSPSNHLDHSSSMNVDKVVQSVTNATKRLSQISTNTNNSNKKRKTQNKIGPWKLGRTLGRGSTGRVRLAKNTTTGQLAAVKIVPKSNFKKLENPKYKRSKEDATRLPYGIEREIIIMKLISHPNIMGLYDVWENKNDLYLILEYIEGGELFDYLIKRGKLQEYEAINYFKQIINGINYLHQFNICHRDLKPENLLLDFNKNIKIADFGMAALEVKEKLLETSCGSPHYASPEIVAGKNYHGAPSDIWSCGIILFALLTGHLPFDDENIRKLLLKVQSGKFNMPPELSFEAKDLITKMLKVNPRERITIDAILTHPLLAKYPEPTVSYSSTTTLDINSINIKQIESVDKIDKEILKNLSVLFHNCDEKTIISRLLSPNRCPEKMFYYLLMKYRNEHLSNSNSFNSSNDVDSARSLPRSTSYVKTTVTDHATGEKHTTVKKIQQSSSIYSNRSLLKKSTSAKGNVLSNITNRPNTPKQFSASSSFNKKKALHSKTQIYASRSRNASSRSLKSNSSTGRNGNNASVTSVNKIPEITGATVLQPIPSMAMNRGDEQQNKTKKNLTGTFGNKSLLNFQLICEEVFENDKENSKPVSKTPVSQLPPPPPPPIETPTSRTNSVKRGKTWSLARRERELAEQVRQRNEARENKLKAEELARKELEQEKKRIAEEKKRLEQQERELDEKQKLQEKQKAALEKLQKHQSAHDFEGLFASNRRSVTDMAPSSGMSSLDPRAHMVSRANTIGSPNLSSSSVNIDENASKVLHKFGIDVAPSPKRFSRASKTSTSKNLSSFLAPTVSRNLSSQLKTSSSKNLAGYLHGTTDTNGSAIAAKKKDDSTNEALTIEEFNAKERTSMSPSISKASVNKRNSNQSSYYRSMFSDNGNDDNVTKVRTGESHLSVQEEEEMDMENAIDEDISLIPNPRFSRFSFGGLLGSNTVANEEGDWTIMNSTLNHSNTVVRGTHNKSSTMLGLGIKMRDTTTIKEDEEFEDEKPFISVPSSEDDEGNTHKNKRGGLRDSGNYDFDEEHSVASTANTEYSDVASQGQQMPGSHTIHQLETELSNFDLLSYRVADIGKVNKHKPSIVDSKETLLKNHSSDEATIEVKEDNNEHDFNDKIKQHYDDNGDSEEDDEDEDEEEEDDDDDDDARSSFEARPHSHNYSLAEITSESPVGGGYESPSIANDFKKSRHSTGIFSTTQFPRSPYVVNNNGDSNKDENSQQQTKHMLNDGHKGLITSPVQDTFGSKKPVESNSLFRRLSLNPNRAAPKAPAPPPPSAPISSAAKANISQPLSSPTKGHNRFSRISIGSKNMLQKEDKSTKSNWFKKFFHSLTTPSAKDQSGNSSSKVASKDIKIIDTSLTAAQLIRVIKYQLELKKIEGSISKVDIDEEFGLISGVIPSKFANGRKLKFKIEVIDLINSSSLHVIKMKGNDKGFQSLVNIVTFIIKKEEQDKISRR

SEQ ID NO: 16 - protein, 1349 aa, Candida albicans
MPHSRQPSISSSIMSQSNHNHPQKIGPWKLGKTLGRGATGRVLLATHQTTGQKAAVKVVSKSELQDEETEKNGDGLPYGIEREIIIMKLLTHPNVLRLYDVWETSKALYLVLEYVEGGELFDLLVERGPLPEVEAIKYFRQIILGTAYCHALGICHRDLKPENLLLDSQLNVKLADFGMAALESNGKLLETSCGSPHYAAPEIVSGLKYHGAASDVWSCGVILFALLTGRLPFDDENIRNLLLKVQAGNFEMPVDEVSREARDLIARMLEVDPMRRISTEKILRHPLLTKYPMSNEDLISEKSLPHPQTGYKSLGSVRNIDKQILSNLTILWNDRPEEEIVDCLLKDGSNPEKTFYALLMRYKHNQEDNTNNNSPKKSTSFNNKVVRSGSKYSLNGTPRRKRASHISVSRPTSFQYKSNPGAGATANRNSVARHSVASSANNSPRKSPYKSPYRSPYRSPYKSPSKRYSYNQSPTKSPYGRRSNSQRQFENEPLKAKPRNIYNEIVDAQSNFSLPPSLPPSLPSKDSRYMIDEPNQPQLQQPALSQVPENPIVDESPDLMQSAKISSGKRNSIIGKNNNNSNSNKRMSKRKSIRASMTTGLKRNSITMKLLSTYAKLSGDDDWEYMDKQTKRTSATFAALCDKIFNQEDYDEEDEQLVDPEEKEAKEYERLMELERKKHEAELKARRELEKKKRRQKRRSILSSKKLSIIVKNDADPNNSEQELVDEGIKQPKRQSKNLTALRALSEGNHASEELTLEDVENLKRRSASQPVPKRRQTPVLTRRPVSRLDPLWQAHENEQLDRAKDALEQEWRDSQKRSSTVSRKKVNRESMISVMDDIVEEDQGRVNRRSTRNTYYERERDYELPEPTVEDSNLTDDYMTEIRKSRLLNSQLNVRDPLNEKRKSEPKTLISNVQIPSVTRKSRNFTTSNKRLSVLSMYSTKESYRDLNSIINSPDENPEQHQNMNKPALRTSIADRLDKAGLAEPEYETETDGEDKVSVIDLDDHLADRRTSYYDGSGKRASRASTTKRYNVHSSSGQRPKSKVPDLPKNDYDDTFVSNSDEVHKRQYKSMVSDESSASDDVFDKIKLPDGKSTKSSIDELANGTSTSGHRKPKIRHSQPGPEMLIPHLNGGIESSQPMSKVRGNNSSGHDDSVPPPPPAHKVNKKPLDDKTNFPPPEVDPKRKGSFFRKLSWGSKKTIENNTNAATNTTTQQQLPSPAESKEEKPKSSFFRWFSSSNTPSAAEIRKFNTILPKHEMSTALFALLNSWSNFGLKDLRNDQVGYYITGAISKHNSFNLKSCKFRIKINQRDFNQKSEIVCVRVKGSKVTTDTLFSEIEKVLLKEGVLDK

SEQ ID NO: 17 - protein, 1029 aa, Saccharomyces cerevisiae
MTLDYEIYKEGGILNNRYQKIEDISEGSYGYVSLAKDVREKRLVAVKYIFKLEDDGQYDGPQDDENDCDSSDCDDDEDTKVDTDRHENENGNASSNNGSSREKKHNLYKHKKSLISSKVKSRLSNNICLEAMYEVDIQTKIGRHQNIAALLDFFDSYIIMEYCSGGDLYEAIKADAVPKKTKSITHIITQIMDAIEYVHNKGIYHRDIKPENILISGIDWTIKLTDWGLATTDKTSMDRNVGSERYMSPELFDSNLDIKERKEPYDCAKVDLWAMGIVFLNIVFHKNPFSIANQSDKSFCYFAANREALFDVFSTMAYDFFQVLRYSLTIDPANRDLKMMRTELQNLSEYTLDDEYYNNLDEGYEETMIDGLPPQPVPPSSAPVSLPTPISSSNKQHMPEFKKDFNFNNVNERKRSDVSQNQNVASGFFKKPSTQQQKFFNQGYNTTLSTHERAKSAPKFKFKKRNKYGRTDNQFSKPVNIEDRKKSKILKKSRKPLGIPTPNTHMNNFFHDYKARDEFNTRDFFTPPSVQHRYMEGFSNNNNKQYRQNRNYNNNNNNSNNNHGSNYNNFNNGNSYIKGWNKNFNKYRRPSSSSYTGKSPLSRYNMSYNHNNNSSINGYARRGSTTTVQHSPGAYIPPNARNHHVSPTNQFLRVPQSTAPDISTVLGGKPSYQEHYTQDSMDSEGDHDSDDVLFTLEEGDHDFVNGMDNLSINDHLPHTTVGSHNEVFVHASTNHNNNGNNNHIDTNSTTNQYHRQYIPPPLTTSLHINNNNNESNELPDLLKSPASSEAHLNLSSGPIDPILTGNIGNRYSHSSDSKELEQERRLSMEQKFKNGVYVPPHHRKSFNLGTQVPPMNMKTSNEATLSVSHNSVNFGGSYNSRRSSANESNPLHMNKALEKLSSSPGAKSSFVGFPKPLLPRNHSSTTIALQNEDVFADSNNDAIIFEDEEYEGESDKMAHGKMEGGDNESSSTSPDERQIFGPYEIYAQTFAGSTHDKKLGAGRKTSIQDEMVGSLEQYKNNWLILQQQD

SEQ ID NO: 18 - protein, 502 aa, Saccharomyces cerevisiae
MLSDCLLNNFRITAQIGSGAYGLVFHVVDILTSREYAVKTVFKSSSMDEFYNKNGLNNNSQVARTTLLQTQLYHFFKSFQKKLFLPSVDLDSILQLTENELNRLPHYREIAFQLRVQSHGNIVKIHQVLESSIATFIVMDYYDRDLFTSIVDDKHFVNHGILIKKVFLQLCSALDHCHRLGIYHCDIKPENVLLDRNDNAYLCDFGLSTKSKYLAPNVCVGSSYYMAPERILYCLNTTTNGIHVDECCSSLPTDTGDIWSLGIILINLTCIRNPWLKAHQKEDNTFQHFANDNNVLKKILPISDELFTVLTKILQLNPYTRIDMKTLMSEVSSLTSFTREGPLSQVPILSSEVYMTHIIRNENLFLSDLSHFSADQEQQQQQQQQQQQVQEQEQEQKQEQIQNQEQAQQQQEEEDAEPESDIPSTYNSDGSMEKYEYTNNHNNSTFLTSSMDSTPYQSDIDDVSASKDCKFQQDTLRNRLLCLQMNFSTLTDGPNEKWLPDY

SEQ ID NO: 19 - protein, 461 aa, Saccharomyces cerevisiae
MMMFHNCRINNYLITSQIGEGAYGLVYRALDIRTDRQYAIKAVVQSYGVSKEADMGNDKIHKNSVKLQKKLAKLFKESKNVVRVPSIDLESIENMSEEDFKKLPHYKEISLHLRVHHHKNIVTIHEVLQSAVCTFIVMDYYPTDLFTSIVDNRHFVTNGLLVKKVFLQICSALNYCHEHGIYHCDIKPENLLLDTEDNVFLCDFGLSTTSTYIKPNVCIGSSYYMPPERISFDGRVSSSKSGGHKLGKVCPSCNGDLWSLGIILINLTCIRNPWLKADKTEDNTYYYFTKDPNILKQILPLSDDFYSLLSKILQVNPKNRMSLQELMKEVSSITSFTNEGPLSKVPPLSKSVYEKFVSPVDNTNENLSPKSYVYMHDSKAAKNLSYTSSSEEEDGIKEGIDDDNGSRSGSFGTLDTDTGLHSSFTSTSCESDNECSKISNKFSLFEKKFNELRMSSSSLTN

SEQ ID NO: 20 - protein, 470 aa, Schizosaccharomyces pombe
MMRENPELLLGQVLGDSLRFVSIIGAGAYGVVYKAEDIYDGTLYAVKALCKDGLNEKQKKLQARELALHARVSSHPYIITLHRVLETEDAIYVVLQYCPNGDLFTYITEKKVYQGNSHLIKTVFLQLISAVEHCHSVGIYHRDLKPENIMVGNDVNTVYLADFGLATTEPYSSDFGCGSLFYMSPECQREVKKLSSLSDMLPVTPEPIESQSSSFATAPNDVWALGIILINLCCKRNPWKRACSQTDGTYRSYVHNPSTLLSILPISRELNSLLNRIFDRNPKTRITLPELSTLVSNCKNLTRRLRPAPLVSSRYLAYQQQQQQQQMNLQQGIQGYPHQGYMPTQNIGFPWPPTPQFVSNWNHCATPTIPVSLQVLTPNSSLKVDPTTPLTAPIHATESFWPSAAAAAAAVHNNANSYMPITPTPYPNNAKIFGYPNQPPLTPIPFTGFVLHPAPVGRAADAVDPSRKSL

SEQ ID NO: 21 - protein, 474 aa, Schizosaccharomyces pombe
MKLLQKKGYKVERPLNKGSYGTVVLAHRLFRTPRCKDLKYAIKCIKKPAYTFLQEVNILRQLSRSRHRNIIHFVESFEDNVYYYVVLEYCPLGDLYECILNNDFPNAKNQPEMIKNIFLQIIDGVAHLHSHGIYHRDLKPENFLLSLSEDGSELVVKISDFGLACRDKISYDFGTGSDRYMAPEQFEEVDGAGYSPRAADIWALGICLLNLIFARNPFTYPHEKDPIFADYMLDAMTLFDVFPTLSQDTYNVLRACLCVSPEKRSLAKTREAVLAVTKWTTDDEELESFVNEEEEFRASDFMPAEDNVRCTQSDREPLRTPSVLTPANTIQRGLLPSKLPALSDVDENISTSSSPRSPASLAPVNNSERSYDSGLGESLNNMHIGKSIATAVPVNTKRSPYSCSAPAIVFPNSIKGNKDHLKFGRSWCDMDEEDEEDIVSFGSNDDFGASDELSSKHIGLADDWNVLSQWNDNS

SEQ ID NO: 22 - protein, 413 aa, Pichia jadinii
MTHTDITGSLINEYRIVKLIGSGAYGLVYQAQNTVTGQQVAIKCISKKSNPSVKKQSDYLTTLLAEHLLERDFSLQGLREMSLKRLSMADNIPCPFVREISIHLQVHQHPNVISIHKILDSQVAVFVVMDYYPEGDLFVNIVDRQVYARSSGLIKDVFIQLIDVISYCHSKGIYHCDIKPENIMCANKGSKVVIGDFGLAVKSKYIQSKTCIGSSYYMAPERLCTMNHSLTRLEYPACKGDIWSLGVILINFCCTRNPWMKACEKDATYSAFKKDPKILMEILDISEELWNILCDCFREEPEERISLFELRDRVLKCRSFTVAGPLSRCDSYEQDMDDALECAVPANESSVGSNGSLDLPMDHIIEYAQYLQTLSSVKNTAAGNYTQNQFVLDNNIMDNVSIMSNKSFNMNFA

SEQ ID NO: 23 - protein, 506 aa, Nectria haematococca
MQHHAIFGYQTPPASPGFDNPKCTIQQPFAVPRHYPTRPLAPEERLGRVLEGTLQLTEILGTGAYGVVYLAVDLKTGGKYAVKCLSKFNADGTQLEPRQFAYQQREIRLHWKASNHANVVQMLKIVNDPDCIYVILEYCPEGDLFLNITERGQYVGKDELSRNIFLQILDAVEHCHNLGIYHRDLKPENILVTDRGDTVKLADFGLATSDDRSEDYGCGSTFYMSPECLDPSARKPYYMCAPNDVWSLGVILVNLTCGRNPWKQASFQDSTYRAYAGSKDFLKTILPLSDELNEILGRIFEPNPEQRITLNELRTRIMACSRFTMPAVSPPTPPASPDHTTQYVSTEDAIIDDYDYDSPLSPASSSDDEGSLTSSGSTIDDLDDDFDQERQMPQTPPEYAPHAFDPEEPKEHQLIYHSQEFVPQKYSGPVPVPVQVPVGVPPQPMLCQPVPVPIQAPVPIQAPCQQHKSYFPIWDMVKYVQHVPILQHHIPFHQQVPFMPTFQGCY

SEQ ID NO: 24 - protein, 315 aa, Candida albicans
DLCYANSIIDYNELHLVLIDFGLAMDSATICCNSCRGSSFYMAPERTTNYNTHRLINQLIDMNQYESIEINGTTVTKSNCKYLPTLAGDIWSLGVLFINITCSRNPWPIASFDNNQNNEVFKNYMLNNNKAVLSKILPISSQFNRLLDRIFKLNPNDRIDLPTLYKEVIRCDFFKDDHYYYAQHQHHHNHNQINNAYNHYQKQPNQARPTANQQLYTPPETTTYNSYASDMEEDEISDDEFYSDEEDEDIEDYEEEEEEYFGNEQQQQQQVTTVNGNFGQVKGTCYYDTKTKTTTYIKPPAAYTLETPSQSVEYC

SEQ ID NO: 25 - protein, 640 aa, Saccharomyces cerevisiae
MSPRQLIPTLIPEWAPLSQQSCIREDELDSPPITPTSQTSSFGSSFSQQKPTYSTIIGENIHTILDEIRPYVKKITVSDQDKKTINQYTLGVSAGSGQFGYVRKAYSSTLGKVVAVKIIPKKPWNAQQYSVNQVMRQIQLWKSKGKITTNMSGNEAMRLMNIEKCRWEIFAASRLRNNVHIVRLIECLDSPFSESIWIVTNWCSLGELQWKRDDDEDILPQWKKIVISNCSVSTFAKKILEDMTKGLEYLHSQGCIHRDIKPSNILLDEEEKVAKLSDFGSCIFTPQSLPFSDANFEDCFQRELNKIVGTPAFIAPELCHLGNSKRDFVTDGFKLDIWSLGVTLYCLLYNELPFFGENEFETYHKIIEVSLSSKINGNTLNDLVIKRLLEKDVTLRISIQDLVKVLSRDQPIDSRNHSQISSSSVNPVRNEGPVRRFFGRLLTKKGKKKTSGKGKDKVLVSATSKVTPSIHIDEEPDKECFSTTVLRSSPDSSDYCSSLGEEAIQVTDFLDTFCRSNESLPNLTVNNDKQNSDMKTDRSESSSHSSLKIPTPIKAMIRLKSSPKENGNRTHINCSQDKPSSPLMDRTVGKRTVNNSGARKLAHSSNILNFKAYINSEDSDIRETVEDVKTYLNFADNGQI

SEQ ID NO: 26 - protein, 1142 aa, Saccharomyces cerevisiae
MDRSDKKVNVEEVNVPSNLQIELEKSGTSSSVSLRSPTKSSATNLAGMAEGARDNASIASSSVDSLNMLLERQRVRQLNHPQHQQHISSSLAKTPTTTSSFCSSGSSKNKVKETNRISLTYDPVSKRKVLNTYEIIKELGHGQHGKVKLARDILSKQLVAIKIVDRHEKKQRKFFTFIKSSKISENDKIKREIAIMKKCHHKHVVQLIEVLDDLKSRKIYLVLEYCSRGEVKWCPPDCMESDAKGPSLLSFQETREILRGVVLGLEYLHYQGIIHRDIKPANLLISGDGTVKISDFGVSLAASSTNSSDSSESLDELELAKTVGTPAFFAPEMCLGEDAFTRYNLTKENLFRGSCISFMIDIWAVGVTLYCLLFGMLPFFSDFELKLFEKIVNDPLKFPTFKEIQSNKVSKVSCEEEYEMAKDLLLKLLEKNPQKRMTIPAIKKHPFVSWDFDHVPENDEKLLSSVLEQKLRFQCNQTDQFEPISISKHELKNAVSGVGKKIKESVLKSIPLKDPSDLSNKNYLHPTETTRGRGDANVIVSEGSVLSNIKELSANDGCLNTDSDTNININDDDHYSGDDNDGHLTKRELERELNKFDDKHEAGNMVNLPINSSFASLDSFYIDNFAMARMGMSSPEAGDSVSSVPNLPSAPSSTRLGRSPVFSGVTNQPSPIRPVLPQQKSSFCATGRYDKSHNSLLRNSSSHLTSYNSGRPSSRTGRMNSRNQNLPKIPNSLSKISTTKLTELRVPKDSEIPSPAKNPNADRLRRFPVKKNTKTPAIKDPPRININSSDKSGSKNSPIKSLYQRMKQSKDNSKTFEVRRGNFFSHFNGDDDDSSSQSSVTSSGSESDSELSSTSSSCTSGTQSRNSSNNNAYSETESLPFEFGVDSEDGSGVLLRDLPNEDQIRPFLDIQPCRRMKVKSSLNLEPPSVSSSSSSSSDEDELILNVGTAGHRRRHNSSKLSELSNSPQKGSNNFMYSNGSVHDSETTITPQNMDDLTLHQALSRSQPISKPGPLVLPKRLDQKKATTETSNLTDIVEFNGNNDHRKDKNFDKVLYSRDLLKDALSSTNAGRRRSIPSNKIRGRKDASITMSTNVGNDEHARNTSCHGDKGQENGAIKQRTHERSRSLTVAELNEEKRRSALP

SEQ ID NO: 27 - protein, 559 aa, Saccharomyces cerevisiae
MVLLKEPVQPLPRSSLLYNNASNSSSRIKETRKVKLLYNPLTKRQILNNFEILATLGNGQYGKVKLARDLGTGALVAIKILNRFEKRSGYSLQLKVENPRVNQEIEVMKRCHHENVVELYEILNDPESTKVYLVLEYCSRGPVKWCPENKMEIKAVGPSILTFQQSRKVVLDVVSGLEYLHSQGITHRDIKPSNLLISSNGTVKISDFGVAMSTATGSTNIQSSHEQLLKSRALGTPAFFAPELCSTEKEYSCSTHEIWSLGVTIYCLLFGKLPFNANSGLELFDSIINKPLEFPSYEEMLNGATSGITMEEYTDAKDLLKKLLQKDPDKRIKLADIKVHPFMCHYGKSDAASVLTNLETFHELKVSPPSSCKRVELVSLPVNSSFASLDSVYMENFDHNNLRTGADRNSTYSPSIYDANTLSPSAYHNIGSRESSYSSFSSFTSSTAFASQISIQDAPAIGDQQCLIGESGSSLRVNSCEFPQYTTMSPVGEYPFESTEASLSSTLTPVGNVPQRIKAHLVEGKSNSKDDLRIEADASLVFEASDAQRTRRRMSLYKL

SEQ ID NO: 28 - protein, 652 aa, Schizosaccharomyces pombe
MGSVNNEEKTLIEPQRLLRKNTWHPEVDDSEVPPSVFPEYPVHKAIQKTSDSFRKRNYSAGDYVIAPLGGEREGSSLTHSWTFQPGKHNQRLYSDNFQEAQRQWKRLQEWGEVKETKKIRKRFDRFSGRKYINHYEIIKELGRGMHGKVKLGRDTVTRELLAIKIIPKTERRPKLGRANASSQKEKVRREIAILKKCVHPNVVRLREVIDDPSSTKVYLVLEYMSGGEVPWTDCDSPVLSISEARQYFRDVVLGLEYLHYQGIIHRDIKPANLLLNSSNCVKISDFGVSYIANAGLNEDNDVELAKTVGTPAFFAPELCWTDLDRPRPKISEAIDVWALGVTLFCLLFGRCPFNASMEYELFDKIVNERLNIPSTPDIGEEGRDLLKRLLCKDPEQRITLVEVKLHPWTLDGLKDPEKWLQNTDPSTVSRVEVSTDEVASAISLVGRLRRKLGKLFRFRRPKARVFDSSSSVPSDSSICRPESSGNSSIGLSASELSDSFNRLAVNESQKDRERKQVHPVEMGRNSSEKKPRCDFGWDYEAFPNDNQDADDACSYNTGDSIPQVSKSINGHFETYSRTSMDTDDVASFESPNAKHEESGMPVVTFRNYENYDANPSNFHPVVPGFVSSPNLHLAGGSDTPIYCIEHSFTPTN

SEQ ID NO: 29 - protein, 1408 aa, Candida albicans
MSTSLSHQELSTEKGQSCPPIPDSNPLNPKSPALRTTSNSTILNSPIETINVNTSSKSNISGESTINGSAYSNSTTVVQPEVFGEAHTTTSTHNSASTRTERNEFPNSHLHQHQQESRNNGESNTPMTSPKHFPTDDLRHSLFYKAGSAAHHKSATSSKQSSTTSLKDGLNNANTYHFQNTFLDNNVMSDLEESPVPNERNPIQDTGLGPRSHATKFGVQSTTSLPTTISQSRLPNSNKSSFFPFKSYTSSPVKETKHVFLEYDPITRRKVLNTYEILREIGKGEHGKVKLARDLINNELVAIKIVNRKSRKERPSLRMRKNSSAPVINEYELKVKREIAIMKKCRHKHIVALREVLDDLNSLKIYLVLEYMEKGEIKWKKLQSDVAKPTANKCYDANDNEIPCCGNGRMQQRQQSLLTDEDLLSNEFSPNLTFKQSRKIFRDVLLGLEYLHMQGIVHRDIKPANLLVSADNIVKISDFGVSFATSLAENDEGYLVNELDLAKTAGTPAFFAPELCQFDDETATEKLSSSTESMAPPKIDYKIDIWALGVTLYCLLFGKVPFNADTEYDLFQVIVKEPLKFPNSIKAFNPPATVTEEEFELAKDLLSKMLDKNNRTRIEIQDIKEHPFTLMDLDNDVDGLHELFHLNGDNPVEPLSFDLDEHDIVSKDEVDNAVIGVGARIKRSLVRAIRAGGLKDGEIRNKFAALQLEHSRSENSEESSSGYSNYSSSTRLLGYQNGQNYSMILSEGLPVSSATPPPALLAAQQKRSSLLSPKSGGISEKNNPHFPSSLAHQIPNTSSPSTCSSSTSMAFQNHFSFAGMRESGKSLLHDMIESNSNNSSRRGSSAGITVSEAPQIETKRNVGGDLYLKNQSVVETFKGIQLQDDKRRRSSIFSLHSQIGTNSNKSSLSHELTPTQTGSGASTQQQHQHYSTNYSNIAAPIPVPAPRKQSTSDNEQDIKAPLLHQEKNVMGKPYLKIGPISIAREDEKSADDHPDSSIISLPLSESFASLDSINDDYLSRKYEEYTNNRKENSKSEGNVPVISLRRKSSLSESDLTRHVQLKDPFGKFKPDGSEIAEKFKAFNLGNLMKTGGKFALAEHNSSDSQQIKGNNYQSSDDGIAPKAIVPAISYSSSDSYSSCSSSSFDDEDESDDDEENLTLAFQSKVAPISRANFLSLTGRAKSHDSNLPTLRQNREKGRQLNPEFQGPIIFHDGLPEFEDVPDGLINSNPIGNNNVNGNSAFNSGYSYYDNVNPTVVSSNVSTATLTMGMAPSESVETNVEAPAQENAVKVSSPLNPHKNTSSRSDILKDLKKNTSISFGEILFNDQFNNHYKKDPVYSPFPSAKHLDNDQETIVKESASKFHEHRPTYYRSNSVTIGLLHRSTHREDDDDVSQTGNDLEQLITKEKQG

SEQ ID NO: 30 - protein, 855 aa, Saccharomyces cerevisiae
MGDEKLSRHTSLKRARSLSESIKGLFKPSGISGSNNAAAPSSRPGQDQAHSHQTARIITSNVSSPSISPVHSPVLQAAPKHHKLGVPNIAKLSLSPSREPSLNSENEMFSQESFISEKDEDEANLLEREDLQNKKEEKARAKHVRSKEAYVPHHRYTVGSDEVERQPRERLKNFPQNAGSSNPANSNANHVLDQENNFSIDAMLDYDEESKLRRRNSLGVRNHSNRTRSRKNSLSTPRSPPMKNGNGGMNSNATNNVGNGTGNRIYMRGRNHSDSISASSLPKFQEIECKCILDLGHFKVFENGYHEHSLRVLPIITNNKNVDSGDEKDADASVNSGDDGDNDSEANMHKQKSVFSLSGLFKSHKDGNQQQQQQQQQEENGEQINLEKAFSIIPSQRFIKSQTLKKSRTSNLKNGNNDELMKNDGKNIPQIVNPNAAVGVEELKLINALSEKIRKGLKSENTKGNNGEGRSNSNKQEDSDDTEGKAGTTNDDTSHKPCSQKYGKSIGVVGAGAYGVVKICARCKTAKDVLPYSTYSNGKKLFFAVKELKPKPGDQIDKFCTRLTSEFIIGHSLSHPHFEANAMIAGNVSRTTPPKHVFNAPNILKILDLMEYSNSFVEVMEFCASGDLYSLLTRNNISNESNNGSSRLIQTVKEGSGSPLHPLEADCFMKQLLNGVQYMHDHGIAHCDLKPENILFQPNGLLKICDFGTSSVFQTAWEKHVHFQSGAMGSEPYVAPEEFIRDAEYDPRLVDCWSCGIVYCTMVMGQYLWKIAIPEKDSLFKSFLSEIKDDGQFYLFEELRHVSSELNRLRKIALYRTFQVDPTKRITIEQLLQSSWMRKTKCCVVYRHLHTKVSK

SEQ ID NO: 31 - protein, 734 aa, Saccharomyces cerevisiae
MMDSKTLTASMVMQEEKKRQQPVTRRVRSFSESFKNLFRPPRSRDSSPINVTRIPYRSSSTSPKRSSEPPRRSTVSAQILDPKNSPIRQRSYTLKCCTPGLSHPFRQTGSGASNSPTRHRSISGEEQEIVNSLPEYKRSASHTFHGIRRPRSRSSSVSSCDSSNGTTSSSDSQWAMDSLLDDSDNDLTPYRGSNKDILKSKDRAPYNYIDDYNKKALRRATSYPNPLPSKQFYNERLYTRRSHPDEESLESLPRFAGADVQCIIEQNGFKVYEDGSHEHNIKLSGVIAKLEKGNSLPAHRQGSLSRPRLGITLSGLFKHHKNECDIENALSLLPNVEKSQTNHEKRTGQSPNDSNRSSPTQGREDYLKIVNPDASLGSDELKLINSLSSRIHKSLQNYLQEKNLKPAECIGEQAPTFQDNYGHPVGLVGAGAYGEVKLCARLRNEKDSPPFETYHDSKYIYYAVKELKPKPDSDLEKFCTKITSEFIIGHSLSHYHKNGKKPAPNILNVFDILEDSSSFIEVMEFCPAGDLYGMLVGKSKLKGRLHPLEADCFMKQLLHGVKFMHDHGIAHCDLKPENILFYPHGLLKICDFGTSSVFQTAWERRVHAQKGIIGSEPYVAPEEFVDGEYYDPRLIDCWSCGVVYITMILGHYLWKVASREKDMSYDEFYKEMQRKNQFRVFEELKHVNSELATNRKIALYRIFQWEPRKRISVGKLLDMQWMKSTNCCLIYDST

SEQ ID NO: 32 - protein, 789 aa, Saccharomyces cerevisiae
MSSLTRLLQEKRKNETSNSSPRTSADTLTTTPESQSLDLHSRNKSSSHIGSVSNSSSSDRNRANVPVPGSVTTVTQIYSEEDSSSTAGSSLDDRNQFSSSFLNANFAHTASFYGTSAQSRDRFGSLINDQGTAGLSSHGGSFAAQNRITSRLSTTSHTSGRAIPSLSSSIPYSVPNSNKDNNSSNSNSSSLSSSWLETYAGGMPNNITHESNVISSPKVDSVEPRFVISKQKLQKASMDSNNANATQSRSISRSGSFSSQLGNFFFSKNSKESSNSNSAGMSFSANSNGPSPNIKNPNVTNGSTPIPKPIRARQSSIYSASRQPTGSYTDNFYGSPSSVHDHLPPSQSVPRSQHSSIGDLKRFFKKSSNSNLSSNSNNVIPNGSPLSSGIAVPSHSHSSSHFAAGNNSYSTSYNGNGDTIYSHSHGGSGIPFSKRYIKTGADLGAGAGGSVKLAQRISDNKIFAVKEFRTKFENESKRDYVKKITSEYCIGTTLNHPNIIETIEIVYENDRILQVMEYCEYDLFAIVMSNKMSYEEICCCFKQILTGVQYLHSIGLAHRDLKLDNCVINEKGIVKLIDFGAAVVFSYPFSKNLVEASGIVGSDPYLAPEVCIFAKYDPRPVDIWSSAIIFACMILKKFPWKIPKLRDNSFKLFCSGRDCDSLSSLVTRTPDPPSYDESHSTEKKKPESSSNNVSDPNNVNIGPQRLLHSLPEETQHIVGRMIDLAPACRGNIEEIMEDPWIRSIDMCHLVEDGLSFKVVRGEDHHHTQVDQSEAHIAGLEKKKKKQNNQ

SEQ ID NO: 33 - protein, 699 aa, Saccharomyces cerevisiae
MSLSRILRYNQRNNKTTASLTAEHAYSDNWAYSVSLGDPTSVGVNMAAKTGEALNKSYDSVFSSLPVADSVPRTDFTASSRDDENTDVQKLTTSWMEKIDTKMPENISKIDSNIISSPMVSKVEARFIVPKGRLRKNSTDFTSSFSNSLSLPKSYGKLIFFTSKKNSSSTKKNLANDISDNKHNNNSSNTIGHNIPVTTATATCDEIACTSTEHEYNVYEEERMFTTRVYSLEDSVSSLSTNPLDDTYSEAVQVNTRHIEDTESTAHIRKHSYTTSLSSIKRLFKITSFSNNNSNSCDHQESTVADDCAISSSLKETTSSPVSTGSFSLMIENEDSDRDQIIQALYSNIEASTDLVSRKYRDLDVVLGEGSGGKVKLVQRVLDNKVFALKEYRSKKKRESERKYIKNIISEYCIASTLKNPNICETLEILYEKGKIFQILEYCEYDLFSLVMSEKMHYEEICCLFKQLINGVKYLHDIGLSHRDLKLDNCVVTRRGILKLIDFGASSVFHYPLSSQMIEANGIVGSDPYLSPEVFYFNEYDPRALDVWSVGIIFFCMITRRFPWKYPKVKDVQFKAFCSGRGVSSFKDLVTRPATDDSNNYDNDGYEEGVIDMGPNFILHRLPEETHKIMRRILEVSPFRRITINGILQDGWIKEIETCQVVGAASPNEASLRIINKGNHIHTNIDQRYAHIGGLHQRT

SEQ ID NO: 34 - protein, 560 aa, Saccharomyces cerevisiae
MTVSHNHSTKISQQPISSVSAFKFFGKKLLSSSHGNKLKKKASLPPDFHSTSTNDSESSSPKLPNSLKTSRRANSFAHTTNSKRSLSSASTKILPPAGSSTSISRGNRHSSTSRNLSNSKFSSERLVYNPYGVSTPSTSLSSVSTSMKKDPDLGFYLHDGDSKIRMLPIPIVDPNEYLPDEMKEASIQLSDNFVFDDENKTIGWGGSCEVRKIRSKYRKKDVFALKKLNMIYNETPEKFYNAAPKEFIIAKQLSHHVHITNTFLLVKVPTTVYTTRGWGFVMELGLRDLFAMIQKSGWRHVALAEKFCIFKQVACGVKFCHDQGIAHRDLKPENVLLSPDGVCKLTDFGISDWYHHGSTRPVQPCQEVRRDDRLAPYAPPEVMFYDSKKHYDTELQQPYDPRALDCYGLGIILMTLVNNVIPFLESCSFDTGFRDYCDAYENFIRLHDRAFRNRAITARGREWSITWLEISRTDMHLAWHGGSLTQKPPPATPSTTSSKTHGSKELRLVLMPTTNMCVRNLLSKPLRTRIRGVSISLQMLLQPHPPQTHSLRTESPSGQW

SEQ ID NO: 35 - protein, 818 aa, Saccharomyces cerevisiae
MAGNGKDKEVDKSPSVSTLKLLGKRLFNSSSHTDNSSLLLSAEQLGNGRSLRKRPTSPSISGSGSGGNSPSSSAGARQRSASLHRRKNNASVGFSNGSVSSHKSSVALQDLIKHNNNPYLNSPSDILGTGTGIASTRDRDRAVLDREKEKERARNKERNTHHAGLPQRSNSMASHHFPNENIVYNPYGISPNHARPDTAFADTLNTNKENDLSFYMHDGNSKIRMLPLPIANPNDFLPEDMKQYSVHLTDNFVFDTDNKPIGSGGSSEVRKVKSSYRQKDVYALKKLNMIYHESPEKFYKRCSKEFIIAKHLSHNVHITNTFYLLKVPTTTYTTRGWGFIMELGVKDLFQLMERTGWKNVPFNEKYCLFKQVAQGIKFCHDNGIAHRDLKPENVLISKEGICKLTDFGISDWYHVIPHDYTSPVKTCQGMIGSPPYTPPEVMYFDAKKHYPEKFQKPYNPLAMDSYALGIMLITMINNIIPFIDSCNTDARFREFEVSYDNFINHQNPHFRDKGCHKPGPGSEYSLARNFKNTDATRIAWRLADPNPATRYTMDDLFNDPFFQQIETCVEPNDDDLVRVPELRKSTSTNDFSENSLDAPHDQEVIHTSNPFLKKETLTSKPRSMLEIAESPSLKQKSKVKDSAKTKTHDVGDEGGNESTKPKQQDKKENLKKDEVKNGDKDKVIEEATTTNVDSILEKPTPTSTKVEDNLSEDDSTMKELKSMLNSTPTTPTHNGPTPLPAKAGTQLDKRMSDLSLKSETPASTKNFSAPNVSSSSNSLRSLGSPSVSSSKKKKVIHHHLDITNSVTNMSSVSAFISR

SEQ ID NO: 36 - protein, 603 aa, Saccharomyces cerevisiae
MTGMNDNNAAIPQQTPRKHALSSKVMQLFRSGSRSSRQGKASSNIQPPSNINTNVPSASKSAKFGLHTPTTATPRVVSNPSNTAGVSKPGMYMPEYYQSASPSHSSSSASLNNHIDINTSKSSSAASLTSSVSALSLSPTSAINISSKSLSPKFSHHSNSNTAITPAPTPTASNINNVNKITNTSAPICGRFLVHKDGTHEHHLKNAKRQEKLSTMIKNMVGASKLRGEAKSAVPDIIMDPKTTLKSNKNPPTLFAGFMKQVVDMDDKYPEGAPTSGALNCPERDIYRSDQKDSKNNTHNITTTKKDRQCFAEKYGRCQEVLGKGAFGVVRICQKKNVSSQDGNKSEKLYAVKEFKRRTSESAEKYSKRLTSEFCISSSLHHTNIVTTLDLFQDAKGEYCEVMEYCAGGDLFTLVVAAGKLEYMEADCFFKQLIRGVVYMHEMGVCHRDLKPENLLLTHDGVLKITDFGNSECFKMAWEKNIHLSGGVCGSSPYIAPEEYIKEEFDPRPVDIWACGVIYMAMRTGRQLWSSAEKDDPFYMNYLKGRKEKGGYEPIESLKRARCRNVIYSMLDPVPYRRINGKQILNSEWGREIKCCHNGRALK

SEQ ID NO: 37 - protein, 620 aa, Saccharomyces cerevisiae
MVKETPLHSSSSTSLSSLFRPTKLKNLSAKIFNGGGNQSYSKTDDVSRSSSRSSKKNTDSDQEDQIKYNKPNDRRSTIGKSPQGNGALSKESHVVASSTLTGISPTSAKKAPIDYSPSRPLPNNHNPVRTGHTVPHLPHSIHNPINYIHQGSKDAFHHPHPVRSTAHSNISTVSSAKSDTPSSNLSYQAHMHPVEILQKQIEDKHFMDSQASTPGSVELQHNSSSGSDDTSSRKKKSLRLTRFFKKIHNDYHDNHHHHHHHNRGSTPTKPKLNLNTNENIVESNGKALYETDNPVELLEKYGIPGRKLGEGASGSVSVVERTDGKLFACKMFRKPHLNNEGTNQSQLANYSKKVTTEFCIGSTLHHENIVETLDMLTEGDTYLLVMEYAPYDFFNLVMSNLMTQDEVNCYFKQLCHGVNYLHSMGLAHRDLKLDNCVVTKDGILKLIDFGSAVVFQYPYEDTIVKSHGIVGSDPYLAPELLKQTSYDPRVADVWSIAIIFYCMVLKRFPWKAPKKSFNSFRLFTEEPEDEDDIVRGPNKILRLLPRHSRTIIGRMLALEPKQRVLMNDVVKDDWLVSVPSCEVDPTSGDLVEKPKNHKHHLVTEEELNELTKQHGNKDSN

SEQ ID NO: 38 - protein, 759 aa, Saccharomyces cerevisiae
MPNLLSRNPFHGHHNDHHHDRENSSNNPPQLIRSSKSFLNFIGRKQSNDSLRSEKSTDSMKSTTTTTNYTTTNLNNNTHSHSNATSISTNNYNNNYETNHHHNISHGLHDYTSPASPKQTHSMAELKRFFRPSVNKKLSMSQLRSKKHSTHSPPPSKSTSTVNLNNHYRAQHPHGFTDHYAHTQSAIPPSTDSILSLSNNINIYHDDCILAQKYGKLGKLLGSGAGGSVKVLVRPTDGATFAVKEFRPRKPNESVKEYAKKCTAEFCIGSTLHHPNVIETVDVFSDSKQNKYYEVMEYCPIDFFAVVMTGKMSRGEINCCLKQLTEGVKYLHSMGLAHRDLKLDNCVMTSQGILKLIDFGSAVVFRYPFEDGVTMAHGIVGSDPYLAPEVITSTKSYDPQCVDIWSIGIIYCCMVLKRFPWKAPRDSDDNFRLYCMPDDIEHDYVESARHHEELLKERKEKRQRFLNHSDCSAINQQQPAHESNLKTVQNQVPNTPASIQGKSDNKPDIVEEETEENKEDDSNNDKESTPDNDKESTIDIKISKNENKSTVVSANPKKVDADADADCDANGDSNGRVDCKANSDCNDKTDCNANNDCSNESDCNAKVDTNVNTAANANPDMVPQNNPQQQQQQQQQQQQQQQQQQQQHHHHQHQNQDKAHSIASDNKSSQQHRGPHHKKIIHGPYRLLRLLPHASRPIMSRILQVDPKKRATLDDIFNDEWFAAIAACTMDSKNKVIRAPGHHHTLVREENAHLETYKV

SEQ ID NO: 39 - protein, 636 aa, Schizosaccharomyces pombe
MGEKDKLHEISSKFASLGLGSLKSTPKARETTEPPPPSSQQPPSTPNGKEAASPSALKQNVRPSLNSVQQTPASIDAVASSSNVSLQSQQPLSKPVVSSKPNQTTAMPPPSNNPSRHVSSTSNKPAAVSPNPAAHHAELPSGSVPPSASVSRANSTATTTPHKAGVVSNPAAANVHVLSVAASPNPSTPSNGPAPVSTTATPSRNPVTRLQRIFSQNSVSRQNSRTGRGAAVANTEETNSTGGSETGGAANSSSTSNPSSAKWSRFTVYDDASHTHQLRPARRQEKLGKMLKDFLAGNSKKREEERIAKEAADAQHQLSLVQSWINGYGQEKLADKKDPAKVSASFVEKYGRCQEVIGRGAFGVVRIAHKVDPQNSGSETLYAVKEFRRKPAESQKKYTKRLTSEFCISSSLRHPNVIHTLDLIQDGKGDYCEVMELCSGGDLYTLIMAAGRLEPMEADCFFKQLMRGVDYLHDMGVAHRDLKPENLLLTVSGSLKITDFGNGECFRMAWEKEAHMTCGLCGSAPYIAPEEYTESEFDPRAVDVWACGVIYMAMRTGRHLWRVAKKSEDEYYSRYLMDRKNESGYEPIEMLERSRCRNTLYNILHPNPTYRLTAKQIMKSEWVRSITLCEAGNAGL

SEQ ID NO: 40 - protein, 650 aa, Schizosaccharomyces pombe
MSVTPPNVQFNLNGDSDHKSDNSSSSLENKLDTELKITSPPRNPPQRLHPVDFSEHADTDDDMNHPLPRVQSPVHIKNHIDPKLAEDRYRSSAARHFEPISIPPSAITSEDEDDYHGSANSSTVLPPRTENALHAASPKPSGSTGYTSPALSQNSGSGGEGESDEGSFNTQHHRSPIFQAYPSSEDLVGDPNDPYRRTRRAPIKTNPHDIPSQFIFRKLGLHHGKHGHHGHSGSLSLKSLVPNHHDKHDKHDKHEKHHSSLDLRRFFKSHQKTDKEKKPSVSKSKSSANLQDDHFGLFKKYGKFGRMLGSGAGGSVRIMKRSSDGKIFAVKEFRARRPTETEREYARKVTAEFCIGSALHHTNIIETLDIVEENKKFYEVMEYAPYDMFSIVMSGKMTMPEVYCCFKQLLSGVAYLHSMGLAHRDLKLDNLVVDSNCFVKIIDFGSAVVFKYPFEADIVEATGVVGSDPYLAPETLVRKLYDPRAVDIWSSAIIFCCMALRRFPWKYPKLSDNSFRLFCMKQPSNDAESPSDILADIKKQRLVEQGCEPIRKTDESHSPNSKTDNSSTHKQELYGPWRLLRLLPRETRAVIAHMLELDPVKRYDIHRVFADNWINDISMCHMENGKVIHSPTHVHNLVASEESPAPPAKH

SEQ ID NO: 41 - protein, 812 aa, Candida albicans
MTKEHSIRNIFKKDKTPDNGSATATPSSSHTGLSKLFHKESKPITPPMKRTPSVSSLKRRNTNPSQTSGISLNHNHHHHQDSQNHNDATTSGGNIHSSTPVNRSRSNSDRLGHVPPTGRKVLSKAETFTHLQQLDTRNAAKNQLRNHRIPSNHLSSPLSAAPHSDKIVYNPYGLNKTATQERPKNTSFYLSGVNDGERVLSNPVASPNDYLPAELQQQHVNLLEDFEIDVGTKKLGDGGSSDVRIINSCHHKKDLYALKKFTLLSKETDEDFYKRVSEEYKIHRKAAISRHVVDAFAILRIQSQSNLTRGWGMVMEFCGGGDLFSVIVKPGWKSTPLAEKYCLFKQIAYGVKFLHDHDIVHRDLKPENVLLDANGLAKLCDFGVSEFGHEVPEDFSSPVKLSTAYVGSPPYAPPEVMLLKEKSSTEIKAFAYDPFKMDCWGLGMLLFCLVYGGVPFQQSSPNDHAFRDYKFSHKRFCTDHHTFKSNQGYPRGPGSEFKLAAKFENNGASRVAWKLCDPSENTRYTMDMLFDDPWFQSVEMCIYESPDQEVNPFVLPGTGENIDTHSVSGYSSVNNSQAPSRRGTFTSRPVGSGAGSGYNSHDESSNGLSSSFRSMLDLKDIPQKITKTDEPLPSNSSVHSNDSSSARAKSKLDHPSSPGSLLSPSTPALQSIPADRVAQTTSPINASLPAVEESDIEHESESEIQGDETESSGLQVLPPIDDVVAASPSSLTHEPQEQVLDSVESCISLPPNRDQAFAGKDGEMCSLVDLKPAALKSATDLQLGADGMCNLGYKIKKHHHTEVSNVSNSSRR

SEQ ID NO: 42 - protein, 800 aa, Candida albicans
MSSLTKLLHESTSTLASPVLSRNTSEVTFKDQGRRTPEILNISDTVDAKSPGITIDVSKPKPSPIDTDGMNVEPQAVHGFDPSPNTKVSFSSPFSPTSPFTRQSSNSFSSNQAFQNTRGAVASPRYIKNNSVSHSSVFMGGESLSSSIPYSAPGGGRGNPASHSGNTGSLQRENSFSSLNTSDSNSSAHIPNLPNGQPINSIHIQSPQVSASSIDSRFVVSKQRIAQAQAQASLSSSQRSNSQSGLSFFFSQKSKPAVKRDSTTDLGAFYNNSYQDRDAPIVSGSPNSLSSAESTVSYGSSAPTRHNSMANLKRFFKKSTPTTSQPVGTSNLSSSLRSASSGASGAMNIPNSLNGQTNNGYQSPSSFSASTSNTSYSQSPGTNSSSVTRSSTLQNKVNYHERRQSVSGIVNNSQQLPFSKRYHSKNAESLGAGAGGSVRLLTRVSDGLTFAVKEFRAKYQNESKRDYAKKITGEYCIGSTLKHPNIIETVEICYENERIHQVMEYCDFDLFAIVMSNKMSREEINCCFKQILAGVHYLHSMGLAHRDLKLDNCVIDKRGIVKIIDFGSAVVFSYPFTKTLIEAQGIVGSDPYLAPEVCVFNKYDPRPVDVWSVAIIYCCMMLKKFPWKVPKLSDSSFKLFASRGEFIPISEMLKKTPNDMEKSNSNGSSGGGLSNLEDISEALEDEITAGAKQKPSTTGQNGATANGKDHTSSETGANRLLLALPEDCRRLIGRMVELAPACRITIDEVLNDSWLKSVNMCTVEESSPGVFEVIKCEDHEHTQVDQSKAHIAAFEKNKKK

(19) United States
(12) Patent Application Publication
(10) Pub. No.: US 2004/0077524 A1
(43) Pub. Date: Apr. 22, 2004

(54) Method for selectively inhibiting fungal growth

(75) Inventors: Gerard Manning, Menlo Park, CA (US); Sucha Sudarsanam, Greenbrae, CA (US)

Correspondence Address: FOLEY AND LARDNER, SUITE 500, 3000 K STREET NW, WASHINGTON, DC, 20007, US

(73) Assignee: SUGEN, INC.

(21) Appl. No.: 10/618,581

(22) Filed: Jul. 15, 2003

Related U.S. Application Data
(60) Provisional application No. 60/395,624, filed on Jul. 15, 2002.

(51) Int. Cl. C12Q 1/48; A61K 31/00

(52) U.S. Cl. 514/1; 435/32

(57) ABSTRACT

The present invention relates to the isolation of members of fungal-specific kinase families and their use in identifying compounds that modulate kinase activity and the use of those compounds as antifungal agents for therapeutic treatments and medicaments in animals and plants.

### CROSS-REFERENCE TO RELATED PATENT APPLICATIONS

This application is a Non-Provisional of U.S. Application No. 60/395,624, filed Jul. 15, 2002, incorporated herein by reference in its entirety.

### FIELD OF THE INVENTION

The present invention relates to the isolation of fungal-specific kinases and the identification of compounds and compositions that modulate the activity of fungal-specific kinase enzymes and their use as antifungal agents.

### BACKGROUND OF THE INVENTION

More than a million species of fungi belong to the Kingdom Fungi, but only about four hundred are known to cause diseases that afflict humans, animals, and plants. Factors that predispose individuals to the development of fungal diseases include neutropenia, the use of immunosuppressive agents during organ transplantation, intensive chemotherapy and irradiation for hematopoietic malignancies or solid tumors, use of corticosteroids, extensive surgery and prosthetic devices, indwelling venous catheters, hyperalimentation and intravenous drug use, as well as when the balance of the normal flora is altered through antimicrobial therapy.

The majority of such pathogenic fungal species are classified within the Phyla Zygomycota, Basidiomycota, Ascomycota, or the form group Fungi Imperfecti, but in general, are considered as either yeasts or molds. Yeasts are typically solitary rounded forms that reproduce by budding or fission. Mold spores, on the other hand, germinate to produce branching hyphae filaments that may be uninucleate, binucleate, or multinucleate.

Mold-like fungi called dermatophytes cause athlete's foot, groin-related infections and ringworm of the skin or scalp. These fungi live on dead tissues of hair, nails and the outer layer of skin. Poor hygiene, continually moist skin and minor skin or nail injuries increases an individual's susceptibility to fungal infections. Ringworm symptoms are itchy, red, scaly, slightly raised, expanding rings on the trunk, face or groin and thigh. Pets also can transmit the fungus to humans.

Aspergillosis is a name given to a wide range of diseases caused by the fungus, Aspergillus, now also known as Emericella. Members of this genus that cause disease in humans include Aspergillus flavus, Aspergillus fumigatus, Aspergillus glaucus, Aspergillus nidulans, Aspergillus niger and Aspergillus terreus. Three principal diseases are allergic bronchopulmonary aspergillosis, pulmonary aspergilloma and invasive aspergillosis. Furthermore, colonization of the respiratory tract is also common. For instance, colonization of the sinuses and lungs, toxicoses, allergic bronchopulmonary aspergillosis, pulmonary aspergilloma, invasive aspergillosis, pulmonary aspergillosis, CNS aspergillosis, sinonasal aspergillosis, osteomyelitis, endophthalmitis, endocarditis, renal abscesses, otomycosis, exogenous endophthalmitis, allergic fungal sinusitis, and urinary tract fungus balls, are among of some of the other diseases caused by Aspergillus infections. Aspergillus also frequently are secondary opportunistic pathogens in patients with tuberculosis, bronchiectasis, other mycoses and carcinoma. Similarly, Aspergillus fungal infections can be a complication resulting from burns, post surgical wounds and intravenous injections.

Another group of fungi of the genus, Blastomyces dermatitidis, cause blastomycosis-related diseases. The infection is acquired via inhalation of asexual spores. After 30 to 45 days an acute pulmonary disease indistinguishable from a bacterial pneumonia may occur. Most cases become manifest during a chronic and indolent phase that may affect the lungs, the skin, the bones, the genitourinary tract and other reticuloendothelial organs.

Yet another major infectious fungus is Candida, which are thin-walled, small fungi that reproduce by budding. Even though there are more that 150 species of Candida, no more than ten cause disease in humans with any frequency. Of these, Candida albicans causes almost 100% of cases of oropharyngeal candidiasis and at least 90% of cases of Candida vulvovaginitis. When Candida produce invasive candidiasis, infection by the other species of Candida are observed. Invasive means the fungus has infected tissues or the blood. Invasive candidiasis, which is also known as systemic candidiasis, is typically seen in individuals that have reduced immunogenecity or weakened immune systems. Almost any organ of the body may be involved.

Some fungi, however, infect only animals and not humans. For instance, the dermatophyte Microsporum gallinae brings about disease in chickens, but not humans. Ringworm in pets and livestock is not uncommon. For example, the dermatophyte Microsporum canis may cause ringworm in a variety of mammals, such as cats, dogs and humans. Similarly, cryptococcosis occurs in cats and humans.

There are a myriad of other fungi that attack and infect plants, such as crops and trees. For example cruciferous crops like cabbage, cauliflower, canola and rutabaga are susceptible to a number of fungal diseases. Pythium and Rhizoctonia fungi rot seeds and older seedlings; Phoma lingam (Leptosphaeria macutans) often kills seedlings or stunts the growth of surviving plants; Plasmodiophora brassicae is a destructive soil-borne disease which affects nearly all cultivated, as well as many wild and weed members of the cabbage family; Fusarium oxysporum yellows or wilts plants; Peronospora parasitica causes downy mildew. Particularly susceptible hosts include canola, cabbage, broccoli, Brussels sprouts, kale, cauliflower, rutabaga, radish, horseradish, Chinese cabbage and mustards, ornamentals such as stock, wallflower, and aubretia, and many cruciferous weeds. There also exists Common Root Rot caused by Fusarium and Helminthosporium; Septoria Leaf Blotch occurs in wheat and Pyrenophora trichostoma infects Spring wheat. Other fungal-induced plant diseases include gray mould and ghost spot diseases of practically all plants by Botrytis cinerea. Leaf mould is caused by the fungus, Fulvia fulva, also known as Cladosporium fulverum. Powdery mildew is a very common disease caused by the fungus Oidiopsis taurica, also known as Leveillula taurica. Late Blight is a very devastating disease of tomato, potato, and eggplant, caused by the fungus Phytophthora infestans.

Even though fungi and mammalian cells are both eukaryotic cells, the differences that exist between them can be sufficiently distinct that they form the basis of targets for fungal-specific drug interactions. The most convenient and effective approach for treating fungal infections therefore involves administering a drug or compound that targets a unique feature of a fungal cell. In this regard, fungi possess a number of biological traits that distinguish them from other organisms. They possess, for example, chitin, ergosterol, a unique lysine biosynthesis pathway, soluble carbohydrates, unstacked Golgi cisternae and unique microtubules. They also differ from other organisms in a range of biochemical and molecular features such as the regulation of some enzymes and some aspects of mitochondrial codon usage.

Thus, drugs that specifically target fungal cell wall synthesis, fungal DNA synthesis or enzymes in key fungal biological pathways are extremely useful in destroying or eradicating an infecting fungus, while having minimal toxicity, if any, to the affected subject. Accordingly, there exist established and developing drug treatments used to combat and treat a wide variety of immunological responses, symptoms and diseases caused by fungal infection in mammals. To this end, there are, in general, half a dozen or so groups of compounds, drugs and chemicals that have proven useful in treating certain fungal infections. Allylamines, for example, are a group of drugs that inhibit ergosterol biosynthesis. This sterol occurs in fungi, bacteria, algae, and plants, and is converted into vitamin D2 by ultraviolet light. Such allyamines include amorolfine, butenafine, naftifine and terbinafine. The latter agent, for example, acts by inhibiting squalene epoxidase, an enzyme involved in ergosterol synthesis.

There also exist azole-based antifungal agents such as fluconazole, itraconazole, ketoconazole, posaconazole, ravuconazole, voriconazole, clotrimazole, econazole, miconazole, oxiconazole, sulconazole, terconazole and tioconazole. These azole antifungal agents also inhibit the synthesis of ergosterol, but by blocking the action of 14-alpha-demethylase.

Other antifungal agents inhibit 1,3-beta glucan synthase or other enzymes involved in fungal cell wall synthesis. Illustrative of such “glucan synthesis” inhibitors are caspofungin, micafungin, and anidulafungin. There also are agents that target fungal cell membranes, causing the fungus to leak electrolytes. Exemplary of such “polyenes” are Amphotericin B (AmB), AmB lipid complex, AmB colloidal dispersion, liposomal AmB, AmB oral suspension, liposomal nystatin, topical nystatin and pimaricin ophthalmic. Yet other drugs include griseofulvin, which inhibits fungal mitosis; the antimetabolite, flucytosine, which is a DNA substrate analog that leads to incorrect DNA synthesis; and topical drugs such as ciclopirox olamine, haloprogin, tolnaftate, and undecylenate.

Nevertheless, development of new antifungal agents is difficult because there are relatively few key fungal genes and proteins that are not present in the human genome. Therefore, a need exists to identify fungal genes that are sufficiently distinct from any human counterpart that they can form the basis of fungal-specific drug interactions, and specifically be used to identify drugs targeted against fungi. A number of kinase families have now been identified which are present only in fungal genomes.

### SUMMARY OF THE INVENTION

In one aspect of the present invention, a method (“method 1”) for identifying an antifungal agent that inhibits a fungal-specific kinase in a sample is provided. This method comprises determining the activity of the fungal-specific kinase in the sample before and after exposing the sample to a test compound. In one embodiment, the sample is a fungus. In another embodiment, the sample is a preparation of a fungus extract. In another embodiment, the sample is a cell or culture of cells. In another embodiment, the cell or culture of cells may be suspected of containing fungal cells. To this end, the cells may be mammalian, bacterial, insecticidal, or fungal cells. In yet another embodiment, the sample is an isolated and/or purified preparation of the kinase. In a further embodiment, the kinase is recombinantly produced.

In yet another embodiment, the fungal-specific kinase is selected from the group consisting of KIN1, KIN4, GIN4, RAN, ELM and HAL kinases. In a preferred embodiment, the kinase domain of the kinase has at least 46% sequence identity to a kinase domain of any one of SEQ ID NOs. 1-5. Preferably, the kinase domain has a percentage sequence identity to a kinase domain of any one of SEQ ID NOs. 1-5 of 50%, of 55%, of 60%, of 65%, of 70%, of 75%, of 80%, of 85%, of 90%, or of 95%. In another preferred embodiment, the kinase domain of the kinase has at least 55% sequence identity to a kinase domain of any one of SEQ ID NOs. 6-9. Preferably, the kinase domain has a percentage sequence identity to a kinase domain of any one of SEQ ID NOs. 6-9 of 60%, of 65%, of 70%, of 75%, of 80%, of 85%, of 90%, or of 95%. In a further embodiment, the kinase domain of the kinase has at least 55% sequence identity to a kinase domain of any one of SEQ ID NOs. 10-16. Preferably, the kinase domain has a percentage sequence identity to a kinase domain of any one of SEQ ID NOs. 10-16, of 60%, of 65%, of 70%, of 75%, of 80%, of 85%, of 90%, or of 95%. In one other embodiment, the kinase domain of the kinase has at least 55% sequence identity to a kinase domain of any one of SEQ ID NOs. 17-24. Preferably, the kinase domain has a percentage sequence identity to a kinase domain of any one of SEQ ID NOs. 17-24, of 60%, of 65%, of 70%, of 75%, of 80%, of 85%, of 90%, or of 95%. In another embodiment, the kinase domain of the kinase has at least 38% sequence identity to a kinase domain of any one of SEQ ID NOs. 25-29. Preferably, the kinase domain has a percentage sequence identity to a kinase domain of any one of SEQ ID NOs. 25-29, of 40%, of 45%, of 50%, of 55%, of 60%, of 65%, of 70%, of 75%, of 80%, of 85%, of 90%, or of 95%. In yet another embodiment, the kinase domain of the kinase has at least 30% sequence identity to a kinase domain of any one of SEQ ID NOs. 30-42. Preferably, the kinase domain has a percentage sequence identity to a kinase domain of any one of SEQ ID NOs. 30-42, of 60%, of 65%, of 70%, of 75%, of 80%, of 85%, of 90%, or of 95%.

In another embodiment, a reduction in fungal-specific kinase activity after the sample is exposed to the test compound indicates that the test compound is an antifungal agent. In a preferred embodiment, the test compound does not inhibit kinase activity of a kinase endogenous to a non-fungal organism. In a preferred embodiment, the non-fungal organism is a mammal, animal, tree or plant. In a more preferred embodiment, the mammal is a goat, sheep, cattle, horse, cat, dog, pig, rat, mouse, primate, or a human. In a most preferred embodiment, the mammal is a human. In one other embodiment, the non-fungal organism is a fish, bird, or a reptile. In another embodiment, the non-fungal organism is a plant selected from the group consisting of barley, wheat, corn, rice, cotton, oak, tomato, potato, Dutch elm, and Chestnut.

In another embodiment, the test compound identified by method 1 as an antifungal agent reduces fungal growth when applied to a living fungus. In yet another embodiment, fungal growth is reduced in the fungus to which the antifungal agent is applied and/or to fungal progeny. In yet another embodiment, the test compound eradicates a fungus to which it is applied.

In one other embodiment, kinase activity is determined by comparing protein phosphorylation patterns of the sample in which the fungal-specific kinase is present in the presence and absence of the test compound.

Another aspect of the present invention is a method (“method 2”) for identifying a compound that has antifungal properties. This method comprises (i) selecting and culturing a fungus sample that contains a kinase having a minimum sequence identity to any one of SEQ ID NOs. 1-42; (ii) treating the fungus sample with a test compound; and (iii) determining, after the treating of step, the level of activity of the fungus in comparison to an untreated control fungus sample. In one embodiment, a decrease in the level of fungus activity of the treated fungus, compared with the control sample, indicates that the test compound is a compound that has antifungal properties.

In one embodiment of the present invention, a “minimum sequence identity” is the minimum sequence identity that a kinase must have to a kinase domain of the present invention so as to be classified as a fungal-specific kinase.

Thus, in one embodiment, the minimum sequence identity is at least 46% to KIN1, more preferably to any one of SEQ ID NOs. 1-5. In another embodiment, the percentage sequence identity of a kinase to any one of KIN1, preferably to any one of SEQ ID NOs. 1-5, is 50%, 55%, 60%, 65%, 70%, 75%, 80%, 85%, 90%, or 95%.

Similarly, in another embodiment, the minimum sequence identity is at least 55% to KIN4, more preferably to any one of SEQ ID NOs. 6-9. In another embodiment, the percentage sequence identity of a kinase to any one of KIN4, preferably to any one of SEQ ID NOs. 6-9, is 60%, 65%, 70%, 75%, 80%, 85%, 90%, or 95%.

In another embodiment, the minimum sequence identity is at least 55% to GIN4, more preferably to any one of SEQ ID NOs. 10-16. In another embodiment, the percentage sequence identity of a kinase to any one of GIN4, preferably to any one of SEQ ID NOs. 10-16, is 60%, 65%, 70%, 75%, 80%, 85%, 90%, or 95%.

In another embodiment, the minimum sequence identity of RAN is at least 55% sequence identity to SEQ ID NOs. 17-24. In another embodiment, the percentage sequence identity of a kinase to any one of RAN, preferably to any one of SEQ ID NOs. 17-24, is 60%, 65%, 70%, 75%, 80%, 85%, 90%, or 95%.

In another embodiment, the minimum sequence identity is at least 38% to ELM, more preferably to any one of SEQ ID NOs. 25-29. In another embodiment, the percentage sequence identity of a kinase to any one of ELM, preferably to any one of SEQ ID NOs. 25-29, is 40%, 45%, 50%, 55%, 60%, 65%, 70%, 75%, 80%, 85%, 90%, or 95%.

In another embodiment, the minimum sequence identity is at least 30% to HAL, more preferably to any one of SEQ ID NOs. 30-42. In another embodiment, the percentage sequence identity of a kinase to any one of HAL, preferably to any one of SEQ ID NOs. 30-42, is 35%, 40%, 45%, 50%, 55%, 60%, 65%, 70%, 75%, 80%, 85%, 90%, or 95%.

In one embodiment, the step of determining fungal activity in the fungus sample comprises at least one of determining chitin content or performing an agar dilution assay. In another embodiment, a decrease in chitin staining or reduction in optical density indicates a reduction in growth of the fungus in the fungus sample. The fungus samples described herein that are treated with a test compound or are used in a screening assay for compounds that inhibit endogenous kinases, can be intact fungi or extracts prepared from a fungus.

The present invention also encompasses the use of a recombinantly produced kinase in a screening assay for compounds that inhibit that kinase. Thus, according to such a method, a recombinantly produced kinase, such as a KIN1, KIN4, GIN4, RAN, ELM, or HAL kinase is exposed to a compound that may or may not affect the kinase activity of that kinase.

Yet another aspect of the present invention is a method (“method 3”) that comprises administering to a non-fungal organism, a compound capable of inhibiting a kinase in a fungus living in or on the non-fungal organism. In one embodiment, the kinase comprises an amino acid sequence selected from the group consisting of SEQ ID NOs. 1-42. In another embodiment, the kinase comprises an amino acid sequence that has minimum sequence identity of at least 46%, 50%, 55%, 60%, 65%, 70%, 75%, 80%, 85%, 90%, or 95% to a KIN1 family member, or more preferably has minimum sequence identity of at least 46%, 50%, 55%, 60%, 65%, 70%, 75%, 80%, 85%, 90%, or 95% to any one of SEQ ID NOs. 1-5. In another embodiment, the kinase comprises an amino acid sequence that has minimum sequence identity of at least 55%, 60%, 65%, 70%, 75%, 80%, 85%, 90%, or 95% to a KIN4, a GIN4 or a RAN family member, or more preferably has minimum sequence identity of at least 55%, 60%, 65%, 70%, 75%, 80%, 85%, 90%, or 95% to any one of SEQ ID NOs. 6-24. In yet another embodiment, the kinase comprises an amino acid sequence that has minimum sequence identity of at least 38%, 40%, 45%, 50%, 55%, 60%, 65%, 70%, 75%, 80%, 85%, 90%, or 95% to an ELM family member, or more preferably has minimum sequence identity of at least 38%, 40%, 45%, 50%, 55%, 60%, 65%, 70%, 75%, 80%, 85%, 90%, or 95% to any one of SEQ ID NOs. 25-29. Furthermore, in another embodiment, the kinase comprises an amino acid sequence that has minimum sequence identity of at least 30%, 35%, 40%, 45%, 50%, 55%, 60%, 65%, 70%, 75%, 80%, 85%, 90%, or 95% to a HAL family member, or more preferably has minimum sequence identity of at least 30%, 35%, 40%, 45%, 50%, 55%, 60%, 65%, 70%, 75%, 80%, 85%, 90%, or 95% to any one of SEQ ID NOs. 30-42.

In a preferred embodiment, the compound does not inhibit a kinase endogenous to the non-fungal organism.

In a preferred embodiment, the compound reduces growth of the fungus or eradicates the fungus living in or on the non-fungal organism. In another embodiment, the compound is administered to the non-fungal organism by spraying, injecting, ingesting, inhaling, swallowing or applying a topical cream, gel, liquid, powder, pellet, aerosol or fluid suspension containing the compound to the fungus living in or on the non-fungal organism.

In a preferred embodiment, the test compound does not inhibit kinase activity of a kinase endogenous to a non-fungal organism. In a preferred embodiment, the non-fungal organism is a mammal, animal, tree or plant. In a more preferred embodiment, the mammal is a goat, sheep, cattle, horse, cat, dog, pig, rat, mouse, primate, or a human. In a most preferred embodiment, the mammal is a human. In one other embodiment, the non-fungal organism is a fish, bird, or a reptile. In another embodiment, the non-fungal organism is a plant selected from the group consisting of barley, wheat, corn, rice, cotton, oak, tomato, potato, Dutch elm, and Chestnut.

Yet another aspect of the present invention involves identifying kinases (“method 4”) that are fungal-specific and which can be targeted by compounds that inhibit their activity. This method comprises comparing the amino acid sequence of a protein with the kinase domains of SEQ ID NOs. 1-42. In a preferred embodiment, the protein belongs to a fungal-specific kinase family of the present invention if the protein contains a sequence that has a minimum sequence identity (as defined above) to a kinase domain of any one of SEQ ID NOs. 1-42. In a preferred embodiment, the fungal-specific kinase family consists of KIN1, KIN4, GIN4, RAN, ELM and HAL kinase members. In one embodiment the amino acid sequence of the protein is obtained from a public database or a proprietary database. In another embodiment the protein sequence is obtained by sequencing a DNA clone that encodes the protein.

Accordingly, the present invention provides a method for identifying a compound that inhibits the activity of at least one of KIN1 kinase, KIN4 kinase, GIN4 kinase, RAN kinase, ELM kinase, or HAL kinase in a fungus comprising determining the activity of the kinase before and after exposing the fungus to a test compound, wherein a reduction in kinase activity in the presence of the test compound indicates that the test compound is an antifungal agent, wherein the test compound has minimal toxicity to a non-fungal organism, and wherein the kinase domain of the KIN1 kinase has at least 46% sequence identity to a kinase domain of any one of SEQ ID NOs. 1-5, wherein the kinase domain of the KIN4 kinase has at least 55% sequence identity to a kinase domain of any one of SEQ ID NOs. 6-9, wherein the kinase domain of the GIN4 kinase has at least 55% sequence identity to a kinase domain of any one of SEQ ID NOs. 10-16, wherein the kinase domain of the RAN kinase has at least 55% sequence identity to a kinase domain of any one of SEQ ID NOs. 17-24, wherein the kinase domain of the ELM kinase has at least 38% sequence identity to a kinase domain of any one of SEQ ID NOs. 25-29, and wherein the kinase domain of the HAL kinase has at least 30% sequence identity to a kinase domain of any one of SEQ ID NOs. 30-42.

In another embodiment, the kinase domain of KIN1 kinase, KIN4 kinase, GIN4 kinase, RAN kinase, ELM kinase, and HAL kinase has between 80-90% sequence identity to a kinase domain of any one of SEQ ID NOs. 1-5, 6-9, 10-16, 17-24, 25-29, 30-42 respectively.

In any of the methods described herein, the test compound reduces fungal growth or eradicates the fungus. In another embodiment, the kinase activity is determined by comparing protein phosphorylation patterns in the fungus in the presence and absence of the test compound.

In another aspect, a method of identifying a compound having antifungal properties is provided, comprising (a) culturing a fungus sample; (b) treating the fungus sample with a test compound; (c) determining, after the treating of step (b), the level of activity of the fungus the sample in comparison to an untreated control fungus sample, wherein a decrease in the level of fungus activity of the treated fungus, compared with the control sample, indicates that the test compound is an antifungal agent, wherein the fungus sample is a fungus or a fungus extract, and wherein the fungus comprises at least one of a KIN1 kinase, a KIN4 kinase, a GIN4 kinase, a RAN kinase, an ELM kinase, or a HAL kinase.

In a preferred embodiment, the kinase domain of the KIN1 kinase has at least 46% sequence identity to a kinase domain of any one of SEQ ID NOs. 1-5, wherein the kinase domain of the KIN4 kinase has at least 55% sequence identity to a kinase domain of any one of SEQ ID NOs. 6-9, wherein the kinase domain of the GIN4 kinase has at least 55% sequence identity to a kinase domain of any one of SEQ ID NOs. 10-16, wherein the kinase domain of the RAN kinase has at least 55% sequence identity to a kinase domain of any one of SEQ ID NOs. 17-24, wherein the kinase domain of the ELM kinase has at least 38% sequence identity to a kinase domain of any one of SEQ ID NOs. 25-29, and wherein the kinase domain of the HAL kinase has at least 30% sequence identity to a kinase domain of any one of SEQ ID NOs. 30-42.

In one embodiment, the fungus of the present invention is an Ascomycetes, Zygomycota, Deuteromycota, Mycophycophyta, Ascomycota, Gasteromycetes, Myxomycota, Oomycota or Hymenomycetes fungus. In a further embodiment, the fungus is an Aspergillus flavus, Aspergillus fumigatus, Aspergillus glaucus group, Aspergillus nidulans, Aspergillus niger, Aspergillus terreus group, Blastomyces dermatitidis, Candida albicans, Candida tropicalis, Candida glabrata, Candida parapsilosis, Candida krusei, Candida lusitaniae, Coccidioides immitis, Histoplasma capsulatum var. capsulatum, Paracoccidioides brasiliensis, Sporothrix schenckii, Absidia, Apophysomyces, Cokeromyces, Cunninghamella, Mucor, Rhizomucor, Rhizopus, Saksenaea, Syncephalastrum, Mortierella, Basidiobolus, Conidiobolus, Trichophyton, Microsporum gallinae, Microsporum canis mycorrhiza, arbuscular mycorrhiza, vesicular-arbuscular mycorrhiza or Ectomycorrhiza.

In another aspect of the present invention, a pharmaceutical composition is provided for administration to a non-fungal organism. In one embodiment the pharmaceutical composition comprises a compound that inhibits activity of a kinase in a fungus but does not inhibit any kinase that is endogenous to the non-fungal organism infected with the fungus.

Thus, in one embodiment, the compound in the pharmaceutical composition inhibits a kinase that has kinase domain amino acid sequence that has (i) at least 46% sequence identity to a kinase domain of any one of SEQ ID NOs. 1-5, or (ii) at least 55% sequence identity to a kinase domain of any one of SEQ ID NOs. 6-9, or (iii) at least 55% sequence identity to a kinase domain of any one of SEQ ID NOs. 10-16, or (iv) at least 55% sequence identity to a kinase domain of any one of SEQ ID NOs. 17-24, or (v) at least 38% sequence identity to a kinase domain of any one of SEQ ID NOs. 25-29, and or (vi) at least 30% sequence identity to a kinase domain of any one of SEQ ID NOs. 30-42.

### DETAILED DESCRIPTION OF THE PREFERRED EMBODIMENTS

The present invention contemplates the use of compounds that only target and inhibit the function of fungal kinases and not those endogenous to the infected host. Thus, a suitable antifungal agent of the invention is one that destroys, retards the growth of, or decreases the viability of, a wide range of fungi, while having minimal effects upon the infected, non-fungal organism.

Definitions

Antifungal agent: a compound that has antifungal properties is one which destroys or inhibits the growth, reproduction, or other function of a fungus or fungi. An antifungal agent of the invention may exert its antifungal properties upon the fungus to which it is applied, or to subsequent progeny or generations of that fungus. An antifungal agent is also one that inhibits the activity of a kinase endogenous to a fungus or fungal species. Thus, an antifungal agent may exert an effect upon the molecular constituents of a fungus, i.e., a kinase, rather than the macroscopic attributes of the fungus itself. An antifungal agent may be a nucleic acid that inhibits gene expression or causes the degradation of an mRNA transcript associated with a gene product. Thus, the present invention envisions the use of single- and double-stranded DNA and RNA molecules to inhibit gene transcription and mRNA translation. Accordingly, an antifungal agent may act to inhibit or down-regulate gene expression of a fungal-specific kinase by gene silencing, RNA interference or antisense- or sense-technologies. An antifungal agent may include antibodies or peptides. An antifungal agent may also include small chemical compounds such as indolines.

Ascomycetes: these fungi grow as hyphae with cross-walls (septa) or yeasts; sexual reproduction is by fusion of modified hyphae (or yeasts), sometimes by fusion of a “male” spore (spermatium) with a “female” receptive hypha (trichogyne), leading to development of an ascus containing ascospores.

Basidiomycota: these fungi grow as hyphae or yeasts; asexual spores are relatively rare; sexual reproduction is by fusion of compatible hyphae, leading ultimately to production of basidiospores on basidia, sometimes on or in a fruiting body (e.g., toadstool).

Chytridiomycota: typically unicellular, or with primitive chains of cells attached to a food base by tapering rhizoids; sexual reproduction is by fusion of motile gametes; asexual reproduction is by cytoplasmic cleavage in a sporangium, producing motile, uniflagellate zoospores.

Deuteromycota: these fungi grow as hyphae (with septa) or yeasts; sexual reproduction is absent, rare or unknown; asexual spores (conidia) are formed in various ways from hyphae but never by cytoplasmic cleavage in a sporangium.

Eradicat s: to “eradicate” is understood to mean to get rid of completely or destroy, so that no detectable level of the material in question (e.g., a living fungus) remains using standard technology.

External surface: an “external surface” is any surface of an animal or plant that is exposed to, or can be exposed to, the atmosphere, or is not internal of the body or structure of the animal or plant. For instance, skin, hair, eyes, nails, claws, talons, teeth, gums, lips, tongue, the inside of a mouth, hide, fur, scales, bark, stems, leaves, roots, petals, fruit surfaces, or buds.

Fungus: a “fungus” is any of numerous eukaryotic organisms of the kingdom Fungi, which lack chlorophyll and vascular tissue and range in form from a single cell to a body mass of branched filamentous hyphae that often produce specialized fruiting bodies. The kingdom includes the yeasts, molds, smuts, and mushrooms.

Fungal activity: the “activity” of a fungus can be assessed by using methods well known in the art, such as monitoring, for example, its viability, growth status, rate of hyphal development or amount. Fungal activity can be measured before and after treatment with a test compound. Any one of these measurements is an indicator of the relative “activity” of a fungal sample.

Fungal growth: the “growth” of a fungi can be measured, or considered in terms of, its rate of reproduction, hyphae development, or general mass proliferation. For instance, fungal growth can be determined by the rate of apical growth; that is, the rate of growth of the tips of hyphae. The rate of hyphae tip extension, for example, can be extremely rapid and easily quantifiable, growing at rates up to 40 μm per minute. Other assays for fungal cell growth include determining changes in fungal cell mass, volume and number.

Fungal infection: an “infection” is an invasion by and multiplication of a fungus or fungi in or on a bodily part or tissue, which may produce subsequent tissue injury and progress to disease through a variety of cellular or toxic mechanisms. A fungus may also reside in earth or soil.

Infected host: an “infected host” is a non-fungal organism that contains a fungus in or on a bodily part or tissue, that is not normally associated with the host; or a host that contains a fungus associated with a non-disease state of the host but the growth and/or abnormally high level of the fungus, or production of chemicals by the fungus, creates an abnormal or diseased state in the host. Examples of an infected host are, but are not limited to, mammals such as goats, sheep, cattle, horses, cats, dogs, pigs, and humans; fish, birds, or reptiles; plants, such as crop plants, trees, shrubs, ornamentals, and grasses.

Inhibit: to “inhibit” as used herein, means to prevent or decrease the rate of a particular chemical reaction in a fungus or to decrease, limit, or block the action or function of an enzyme endogenous to, or body part of, a fungus. With respect to inhibiting a fungal enzyme, the enzyme may reside within a fungal cell or be in purified or isolated preparation outside of the fungus.

Internal: a fungus may also infect, reside or be present within the body, tissues or organs of an infected host. Thus, an antifungal agent may target a fungus that infects the blood, for example.

Exposing (to test compound): a fungus can be exposed to a test compound by directly contacting any part of the fungus or fungal cell to the test compound. The test compound may be a solid, fluid or aerosol that is in contact with an outer surface of the fungus. Alternatively, a test compound may be injected, swallowed, inhaled, topically applied or infused into the infected host, whereupon the compound targets the fungus in vivo.

Kinase: a kinase is any one of several enzymes that catalyzes the transfer of a phosphate group from one molecule to another. A protein kinase phosphorylates amino acid residues in proteins.

Kinase activity: the “activity” of a kinase refers to the rate of catalytic or enzymatic function of the enzyme in utilizing a substrate or in phosphorylating, for example, a substrate. Those of skill in the art will recognize that there are a variety of methods for determining kinase activity. See, for example, the methods described below.

Minimal toxicity: an antifungal agent that has “minimal toxicity” upon a non-fungal organism is one that does not adversely affect, in any way, the function, life or biological processes of the non-fungal organism. That is, the antifungal agent does not, for example, inhibit the activity of a kinase that is endogenous to the non-fungal organism. Minimal toxicity means that the antifungal agent does not kill or inhibit the growth of the non-fungal organism, even though the antifungal agent may kill or inhibit the growth of a fungus living in or on the non-fungal organism.

Minimum sequence identity: refers to the percentage of sequence identity that is needed for a kinase to be classified to a particular fungal family according the amino acid sequence of its kinase domain. Thus, a kinase is a KIN1 family member if its kinase domain has at least 46% sequence identity to a KIN1 kinase domain, i.e., to any one of SEQ ID NOs. 1-5. Similarly, additional KIN4, GIN4 and RAN family members must have a “minimum sequence identity” of 55% to any one of SEQ ID NOs. 6-24 to be classified accordingly. An ELM family member must have at least 38% sequence identity to an ELM family member kinase domain, as described, for example in any one of SEQ ID NOs. 25-29. A new HAL family member requires a sequence identity of only 30% to any one of SEQ ID NOs. 30-42. Classification of a kinase into one of these fungal families indicates that the kinase most likely does not have a non-fungal analog.

Modulating a kinase: a kinase enzyme can be modulated such that its catalytic or enzymatic properties, or its rate of activity, are reduced or increased in the presence of a compound, such as an antifungal agent. Preferably, the term “modulate” refers to an inhibition of kinase activity.

Non-fungal organism: Examples of a “non-fungal organism” include, but are not limited to, mammals such as goats, sheep, cattle, horses, cats, dogs, pigs, and humans; fish, birds, or reptiles; and plants, such as crop plants, trees, shrubs, ornamentals, and grasses.

Test Compound: a “test compound” is a compound that effects the desired aims of the present invention, i.e., a test compound inhibits the growth of, or outright kills, an infectious fungus and/or its subsequent progeny. Examples of test compounds include peptide mimetics and ATP mimetics.

Toxic: a “toxic” effect is one that is capable of causing injury, retardation of growth or death, for example, by chemical means. An antifungal agent of the present invention should ideally be toxic against a fungus or fungi and not toxic, or minimally toxic, against the infected host. When a fungus is in soil or earth, an antifungal agent should be not be toxic or should be minimally toxic to the environment surrounding the fungus. For instance, a desirable antifungal treatment would eradicate, or inhibit the growth of, a fungus growing on a tree or plant, but would not detrimentally affect the growth of the tree or plant.

Zygomycota: typically grow as hyphae without cross-walls (aseptate); sexual reproduction is by fusion of sex organs (gametangia) leading to thick-walled resting spores (zygospores); asexual reproduction is by cytoplasmic cleavage in a sporangium, producing non-motile spores.

The present invention is directed to the identification of kinases that are unique to fungi by comparing their kinase domains to any of a number of established fungal-specific kinase families. Accordingly, the present invention provides forty-two kinases, belonging to different fungal families, that can be used to identify other kinases that are unique to fungi. That is, additional fungal kinases can be identified based upon their homology or similarity to the forty-two kinases described herein. These, as well as the inventive forty-two kinases can be used to screen for test compounds that inhibit kinase activity. Compounds that inhibit any of these kinases can be used in pharmaceutical or agricultural formulations to inhibit kinases in infectious or undesirable fungi without modulating or inhibiting the activity of a kinase in an infected or non-fungal organism. Accordingly, the present invention provides antifungal agents useful for treating mammals, preferably humans, having fungal infections, as well as agriculturally- and ornamentally-important crops and plants.

In order to determine which kinase families and kinase members of kinase families are unique to fungi, the amino acid sequences of kinases encoded by genes endogenous to the yeast Saccharomyces cerevisiae genome were compared with the sequences of kinases of higher eukaryotes; specifically, Drosophila melanogaster, Caenorhabditis elegans and human. S. cerevisiae sequences that had no close non-fungal homologs were subsequently identified and were classified into fungal-specific protein kinase families. These kinase sequences were then compared to those of Schizosaccharomyces pombe, a fungus that is, in evolutionary terms, separated by a billion years from the evolution of S. cerevisiae. The S. cerevisiae sequences were conserved in S. pombe. Since members of each analyzed fungal-specific kinase family were also found in S. pombe, it is likely that these kinase families are widely distributed in fungi and are broadly required for fungal survival.

The invention contemplates that conserved kinase sequences between S. cerevisiae and S. pombe can be used to design inhibitors to target only these fungal-specific kinases. Since no other close homologs or isoforms were identified in the human genome, such inhibitors would be predicted not to target kinases in human cells.

The yeast S. cerevisiae fungal kinases identified by the present inventors are SEQ ID NOs. 1-2 (belonging to the “KIN1” fungal family); SEQ ID NOs. 6-7 (belonging to the “KIN4” fungal family); SEQ ID NOs. 10-12 (belonging to the “GIN4” fungal family); SEQ ID NOs. 17-19 (belonging to the “RAN” fungal family); SEQ ID NOs. 25-27 (belonging to the “ELM” fungal family); and SEQ ID NOs. 30-38 (belonging to the “HAL” fungal family). Other fungal kinases, which share sequence homology with the endogneous S. cerevisiae kinase families include, but are not limited to those isolated from S. pombe and C. albicans. Thus, the “Kin1-like” (SEQ ID NO. 3) kinase of S. pombe belongs in the same fungal kinase family as the “kin 1” (SEQ ID NO.1) kinase of S. cerevisiae because of their amino acid sequence composition.

Accordingly, kinases from other fungal species can be classified being fungal-specific with no non-fungal homologs by virtue of the similarities in sequence between their kinase domain and any one of the members of the fungal specific families KIN1, KIN4, GIN4, RAN, ELM and HAL described below. KIN1 family

The KIN1 family comprises the Kin1 and Kin2 genes of Saccharomyces cerevisiae, at least two genes (Kin 1 and gi|19113449) from Schizosaccharomyces pombe and orf6.8762 from Candida albicans.

S. pombe mutants of Kin1 display defects in cell polarity and morphology. Mutants in S. cerevisiae Kin2 have delayed entry into stationary phase when nutrients are withdrawn, which may be a pathological condion in natural growth. S. cerevisiae Kin1 has been implicated in vesicle transport within the cell and may be involved in budding or cell wall formation. The null S. cerevisiae mutant is viable; this may be due to redundancy between Kin1 and Kin2, whose kinase domains are more than 90% identical. Thus, an inhibitor of either of these kinases would likely inhibit both, and thus block any vital function redundantly supplied by both genes. Such functions may include response to nutritional stress, cell wall structure or function, and cytokinesis.

The KIN1 fungal kinases include, in S. cerevisiae, kin1 (SEQ ID NO.1) and Kin2 (SEQ ID NO. 2); in S. pombe, Kin1-like (SEQ ID NO. 3) and Kin1 (SEQ ID NO. 4); and in C. albicans, orf6.8762 (SEQ ID NO. 5). The kinase amino acid sequences of these particular family members appear below. Where appropriate, the public database accession number for a kinase is included so as to indicate its source.

The bold, underlined portion of each family member denotes its kinase domain. Accordingly, the present invention allows the skilled artisan to identify and classify newly-identified kinases from other fungi as belonging to the KIN1 family if the newly-identified kinase domains share at least 46% amino acid sequence identity with any one of the underlined kinase domains of an indicated family member listed below. Preferably, a KIN1 kinase domain has a percentage sequence identity to a kinase domain of any one of SEQ ID NOs. 1-5 of 50%, of 55%, of 60%, of 65%, of 70%, of 75%, of 80%, of 85%, of 90%, or of 95%.

| | |
|---|---|
| S. cerevisiae kin1 | |
| MDDYHVNTAFSMGRGNQQDDGNSESNSMHTQPSTMAPATLRMMGKSPQQQQQQNTPLMPPADIKYANNG | (SEQ ID NO. 1) |
| NSHQAEQKERQVELEGKSRENAPKPNTTSQSRVSSSQGMPKQFHRKSLGDWEFVETVGAGSMGKVKLAK | |
| HRYTNEVCAVKIVNRATKAFLHKEQMLPPPKNEQDVLERQKKLEKEISRDKRTIREASLGQILYHPHIC | |
| RLFEMCTLSNHFYHLFEYVSGGQLLDYIIQHGSIREHQARKFARGIASALIYLHANNIVHRDLKIENIM | |
| ISDSSEIKIIDFGLSNIYDSRKQLHTFCGSLYFAAPELLKANPYTGPEVDVWSFGVVLFVLVCGKVPFD | |
| DENSSVLHEKIKQGKVEYPQHLSIEVISLLSKMLVVDPKRRATLKQVVEHHWMVRGFNGPPPSYLPKRV | |
| PLTIEMLDINVLKEMYRLEFIDDVEETRSVLVSIITDPTYVLLSRQYWTLAAKMNAESSDNGNAPNITE | |
| SFEDPTRAYHPMISIYYLTSEMLDRKHAKIRNQQQRQSHENIEKLSEIPESVKQRDVEVNTTANKSEPE | |
| ATLATKDTSVPFTPKNSDGTEPPLHVLIPPRLAMPEQAHTSPTSRKSSDNQRREMEYALSPTPQGNDYQ | |
| QFRVPSTTGDPSEKAKFGNIFRKLSQRRKKTIEQTSVNSNNSINKPVQKTHSRAVSDFVPGFAKPSYDS | |
| NYTMNEPVKTNDSRGGNKGDFPALPADAENMVEKQREKQIEEDIMKLHDINKQNNEVAKGSGREAYAAQ | |
| KFEGSDDDENHPLPPLNVAKGRKLHPSAPAKSVGHARRESLKYMRPPMPSSAYPQQELIDTGFLESSDD | |
| NKSDSLGNVTSQTNDSVSVHSVNAHINSPSVEKELTDEEILQEASRAPAGSMPSIDFPRSLFLKGFFSV | |
| QTTSSKPLPIVRYKIMFVLRKMNIEFKEVKGGFVCMQRFSSNNVAAKREGTPRSIMPLSHHESIRRQGS | |
| NKYSPSSPLTTNSIHQRKTSITETYGDDKHSGTSLENIHQQGDGSEGMTTTEKEPIKFEIHIVKVRIVG | |
| LAGVHFKKISGNTWLYKELASSILKELKL | |
| S. cerevisiae Kin2 | |
| MPNPNTADYLVNPNFRTSKGGSLSPTPEAFNDTRVAAPATLRMMGKQSGPRNDQQQAPLMPPADIKQGK | (SEQ ID NO. 2) |
| EQAAQRQNDASRPNGAVELRQFHRRSLGDWEFLETVGAGSMGKVKLVKHRQTKEICVIKIVNRASKAYL | |
| HKQHSLPSPKMESEILERQKRLEKEIARDKRTVREASLGQILYHPHICRLFEMCTMSNHFYMLFEYVSG | |
| GQLLDYIIQHGSLKEHHARKFARGIASALQYLHAnNIVHRDLKIENIMISSSGEIKIIDFGLSNIFDYR | |
| KQLHTFCGSLYFAAPELLKAQPYTGPEVDIWSFGIVLYVLVCGKVPFDDENSSILHEKIKKGKVDYPSH | |
| LSIEVISLLTRMIVVDPLRRATLKNVEHPWMNRGYDFKAPSYVPNRVPLTPEMIDSQVLKEMYRLEFI | |
| DDIEDTRRSLIRLVTEKEYIQLSQEYWDKLSNAKGLSSSLNNNYLNSTAQQTLIQNHITSNPSQSGYNE | |
| PDSNFEDPTLAYHPLLSIYHLVSEMVARKLAKLQRRQALALQAQAQQRQQQQQVALGTKVALNNNSPDT | |
| MTKNRSPQKEVVPNPGIFQVPAIGTSGTSNNTNTSNKPPLHVMVPPKLTIPEQAHTSPTSRKSSDIHTE | |
| LNGVLKSTPVPVSGEYQQRSASPVVGEHQEKNTIGGIFRRISQSGQSQHPTRQQEPLPEREPPTYMSKS | |
| NEISIKVPKSHSRTISDYIPSARRYPSYVPNSVDVKQKPAKNTTIAPPIRSVSQKQNSDLPALPQNAEL | |
| IVQKQRQKLLQENLDKLQINDNDNNNVNAVVDGINNDNSDHYLSVPKGRKLHPSARAKSVGHARRESLK | |
| FTRPPIPAALPPSDMTNDNGFLGEANKERYNPVSSNFSTVPEDSTTYSNDTNNRLTSVYSQELTEKQIL | |
| EEASKAPPGSMPSIDYPKSMFLKGFFSVQTTSSKPLPIVRHNIISVLTRMNIDFKEVKGGFICVQQRPS | |
| IETAAVPVITTTGVGLDSGKAMDLQNSLDSQLSSSYHSTASSASRNSSIKRQGSYKRGQNNIPLTPLAT | |
| NTHQRNSSIPMSPNYGNQSNGTSGELSSMSLDYVQQQDDILTTSRAQNINNVNGQTEQTNTSGIKERPP | |
| IKFEIHIVKVRIVGLAGVHFKKVSGNTWLYKELASYILKELNL | |
| S. pombe Kin1-like (gi\|19113449) | |
| MKPNTTNLRNECWDTFSIPKRSQNIKINQSTKHQRSISDFVGTAGPGRQVGNWIIKKTIGAGSMGKVKL | (SEQ ID NO. 3) |
| VVNILTGEKAALKMIPFTPNNTSQTVRVQREALLGRLLRHPNICRVIDCIRTPACTYILFEYVPGGQLL | |
| EYILARGKLDEDLARSFAMQLINALVYLHKNFIVHRDLKIENVLLTQDSRQVKLIDFGLSNFYSKDDLL | |
| RTYCGSLYFAAPELLDAKPYIGPEVDVWSLGVVIYVMVCGRVPFDDVSVPMLHSKIKSGKLEFPSYISE | |
| DCCSLIAAMLNVNPRKRCSLEQAAKFPWLKKNSFCLYLPIPLTSIPSTPSIRSHVFKPPFNLKVLQLLH | |
| EHGLASIPELKHELYMAYIERKTTSLVCLYLLGVESLAPALRIPTALPPVYSRHQRHHSEILGAMDLTE | |
| KITAMQCPP | |
| S. pombe Kin1 (gi\|3560139) | |
| MEYRTNNVPVGNETKSAALNALPKIKISDSPNRHHNLVDAFMQSPSYSTQPKSAVEPLGLSFSPGYISP | (SEQ ID NO. 4) |
| SSQSPHHGPVRSPSSRKPLPASPSRTRDHSLRVPVSGHSYSADEKPRERRKVIGNYVLGKTIGAGSMGK | |
| VKVAHHLKTGEQFAIKIVTRLHPDITKAKAAASAEATKAAQSEKNKEIRTVREAALSTLLRHPYICEAR | |
| DVYITNSHYYMVFEFVDGGQMLDYIISHGKLKEKQARKFVRQIGSALSYLHQNSVVHRDLKIENILISK | |
| TGDIKIIDFGLSNLYRRQSRLRTFCGSLYFAAPELLNAQPYIGPEVDVWSFGIVLYVLVCGKVPFDDQN | |
| MSALHAKIKKGTVEYPSYLSSDCKGLLSRHLVTDPLKRATLEEVLNHPWMIRNYEGPPASFAPERSPIT | |
| LPLDPEIIREMNGFDFGPPEKIVRELTKVISSEAYQSLAKTGFYSGPNSADKKKSFFEFRIRHAAHDIE | |
| NPILPSLSMNTDIYDAFHPLISIYYLVSERRVYEKGGNWNRIAKTPVSSVPSSPVQPTSYNRTLPPMPE | |
| VVAYKGDEESPRVSRNTSLARRKPLPDTESHSPSPSATSSIKKNPSSIFRRFSSRRKQNKSSTSTLQIS | |
| APLETSQSPPTPRTKPSHKPPVSYKNKLVTQSAIGRSTSVREGRYAGISSQMDSLNMDSTGPSASNMAN | |
| APPSVRNNRVLNPRGASLGHGRMSTSTTNRQKQILNETMGNPVDKNSTSPSKSTDKLDPIKPVFLKGLF | |
| SVSTTSTKSTESIQRDLIRVMGMLDIEYKEIKGGYACLYKPQGIRTPTKSTSVHTRRKPSYGSNSTTDS | |
| YGSVPDTVPLDDNGESPASNLAFEIYIVKVPILSLRGVSFHRISGNSWQYKTLASRILNELKL | |
| C. albicans orf6.8762 | |
| MNNQDPDSQYHNKKVYPPNLPSIPPPPQQPLSGRPATPRMLRSISGTLKSKTELAHSDKGQESNNETKN | (SEQ lID NO. 5) |
| SNSPHYVPDTHTRQPPPESLKSNIQAPTAVHGNQQKGSLLPPPSIPNPNTMKPAPTPTGVDQPPAKQKP | |
| SPAPKQPQPQQQQQQQQQQQFHRKSIGDWNFVKTIGAGSMGKVKLAQHNATHEICAVKIIPRAAKLYQR | |
| AHANDPPPQTTQEAAQRHKEFEKEVARDRRTIREGALGRLLYHPFICRLYEMVPMTNHYYMLFEYIEGG | |
| QMLDYIVAHGSLKERHARKFARGIASALDYCHRNNVVHRDLRIENIMINEKGDIKIIDFGLSNLYAPKN | |
| LLKTYCGSLYFAAPELLSAKPYIGPEVDVWSFGVVLYVLVCGKVPFDDQSVSVLHEKIKKGNVEYPAFL | |
| SRECVSLLSRMLVVDPTKRASLYEVCSHPWMNKGYDYKVNNYLPRREPLRLPLDPEIIKTIANFELGTV | |
| QGVADELTSILTSVEYQMSCENWYKITETGREYASSQNAQILPDPTGGFHPLVSIYYLVDEMRKRKKAK | |
| EEALKAQRRAQVPTIAVPTPKQQQQQQPQPAQPQPQPQPEVSQPLPEPKPVPPEEIINPAVATQAQANM | |
| TAPKIVETFSETPQRTLDPSKQSVDEKPSAPGPSIAVPEQAHTTSVPSSFVKTQTSIDEDQLSIPEQQS | |
| PRTSTPQTLDPAKVVGGSSGSAISAPNAGSGAGFNSLLRRLSSKKYKGASSPKRSTSPSPNVEGLSPQP | |
| TKADPMVRRGVSMKVTAKEKQTNTRPPKSELIKKKPQHGRSSSTSNKMQGFIPVEYLPPLPTIDTNTNT | |
| IVSDGAKQQNLTVPSTARHMHPTARAKSVGGGHMRKLSYGRVSHGSQNPLPPLPTSMASQNSQEVVGKD | |
| TSEGFFDDVQLDDVGYQEVPQLTESEIIEQYNISKPNSMPSIEHCKTLFLKGFFSVQTTSAKPLPVIRY | |
| NIINVLSKLGVKFQEVKGGFVCMHTPSVQPSHSNELDEENKLYGDAFKSKSSDSFEAAEPEGSKTPSRQ | |
| PSLQLPSHTPTTPSGPKSHKSSNSIGSIGGNVPRRKFSIGNAFNTYRKKNGSQVMMPPNTPATAKVIHG | |
| LYDDDDKERNGEDDDDEDDYGYDDSADSLNGYGGGSDMLISSRIEQRAKHQRTVSSSSQKASKSPLKFE | |
| IHIVKVPLVGLYGVQFKKILGNTWNYKTLASQILNEMNL | |

KIN4 family

The KIN4 family comprises two genes in S. cerevisiae, Kin4 and YPL141 C; one known gene in S. pombe, gi|10185124 (SPAC140.05), and one from C. albicans, orf6.4215. While KIN4 family members are related to the eukaryotic AMP-activated protein kinase (AMPK) family, their sequence similarity is sufficiently distinct so as to distinguish the two families.

YPL141 C is implicated in the cell cycle because the production of its mRNA transcript correlates with control of the cell cycle, i.e., the levels of YPL141 C mRNA peaks at M phase, or at S/G2 phase. See, for instance, Spellman et al., Molecular Biology of the Cell, 9 (12): 3273-97, 1998.

Mutants in both S. cerevisiae KIN4 genes are viable. Due to the high similarity between Kin4 and YPL141 C kinase domains (77% sequence identity and 87% sequence similarity) these two S. cerevisiae KIN4 family members may serve mutually redundant functions. As such, an inhibitor of one gene would likely inhibit both, possibly revealing vital functions of this family.

The KIN4 family members include, in S. cerevisiae, Kin4 (SEQ ID NO. 6) and YPL141C (SEQ ID NO. 7); in S. pombe, SPAC140.05 (SEQ ID NO. 9); and C. albicans orf6.4215 (SEQ ID NO. 8). The kinase amino acid sequences of these particular family members appear below. Where appropriate, the public database accession number for a kinase is included so as to indicate its source.

The bold, underlined portion of each family member denotes its kinase domain. Accordingly, the present invention allows the skilled artisan to identify and classify newly-identified kinases from other fungi as belonging to the KIN4 family if the newly-identified kinase domains share at least 55% amino acid sequence identity with any one of the kinase domains of an indicated family member listed below. Preferably, the KIN4 kinase domain has a percentage sequence identity to a kinase domain of any one of SEQ ID NOs. 6-9 of 60%, of 65%, of 70%, of 75%, of 80%, of 85%, of 90%, or of 95%.

| | |
|---|---|
| S. cerevisiae Kin4 | |
| MASVPKRHTYGGNVVTDRDRHSLQRNNEILHPIHKNQRKHATFGPYIIGSTLGEGEFGKVKLGWTKASS | (SEQ ID NO. 6) |
| SNEVPKQVAIKLIRRDTIKKDADKEIKIYREINALKHLTHPNIIYLEEVLQNSKYIGIVLEFVSGGEFY | |
| KYIQRKRRLKESSACRLFAQLISGVNYMHYKGLVHRDLKLENLLLDKHENLVITDFGFVNEFFEDNELM | |
| KTSCGSPCYAAPELVVSTKAYEARKADVWSCGVILYAMLAGYLPWDDDHENPTGDDIARLYKYITQTPL | |
| KFPEYITPIPRDLLRRILVPNPRRRINLQTIKRHVWLKPHEAFLSIQPNYWDEHLQKERPKPPNKGDVG | |
| RHSTYSSSASSYSKSRDRNSLIIESTLEQHRMSPQLATSRPASPTFSTGSKVVLNDTKNDMKESNINGE | |
| RTSASCRYTRDSKGNGQTQIEQVSARHSSRGNKHTSVAGLVTIPGSPTTARTRNAPSSKLTEHVKDSSQ | |
| TSFTQEEFHRIGNYHVPRSRPRPTSYYPGLSRNTADNSLADIPVNKLGSNGRLTDAKDPVPLNAIHDTN | |
| KATISNNSIMLLSEGPAAKTSPVDYHYAIGDLNHGDKPITEVIDKINKDLTHKAAENGFPRESIDPEST | |
| STILVTKEPTNSTDEDHVESQLENVGHSSNKSDASSDKDSKKIYEKKRFSFMSLYSSLNGSRSTVESRT | |
| SKGNAPPVSSRNPSGQSNRSNIKITQQQPRNLSDRVPNPDKKINDNRIRDNAPSYAESENPGRSVRASV | |
| MVSTLREENRSELSNEGNNVEAQTSTARKVLNFFKRRSMRV | |
| S. cerevisiae YPL141C | |
| MSYTNKRHTYYGGFTNDLSDTFQYPQRTDEQRRKHVTFGPYILGSTLGEGEFGKVKLGWPKNFSNSSNS | (SEQ ID NO. 7) |
| TFDFPKQVAIKLIKRDSISNDYRKEVKIYREINALKHLSHPNIVKLEEVLQNSRYIGIVLEYACGGEFY | |
| KYIQKKRRLKEMNACRLFSQLISGVHYIHSKGLVHRDLKLENLLLDKNENLVITDFGFVNEFCSRNELM | |
| KTSCGSPCYAAPELVISAEPYEARKADIWSCGVILYAILAGYLPWDDDPNNPEGSDIGRLYNYINSTPL | |
| KFPDYILPIPRDLLRRMLVSDPKKRINLKQIKKHEWLKPHSSFLSITPDEWDKLNNTQSVFRLAKPRRR | |
| YGSRPQSSCSTSSLGSRSDKRDSLVIDSTLITFPAPPQESQNHIITRPASIASDQRLSPIRRSNRHNRS | |
| NSAASVALQAVVNADREYVLSHEQSLSPVQNIRQTTGNMTASLSPPPAISPGDIIIETTPIKRNTISGS | |
| SIVPSLEEESSTTMQTSKIQPNNMASSQNHQYNKNKTQNSLQSAKNFYRTSSSSHTKPRPTSYHPGSYT | |
| TPPYNSNTLSIYEINEKAKSSASSQTLNQRDTSPFDSTPYLALDTCITSSSSIESSPKLITHGQFSVAK | |
| PSVDLQSVSGDLIKYKRDADVVTRIYDEKYKQKRKSLRYSGIFSDISCDTVTEESDELRPPESPLQQHE | |
| GQESIDKAKTEDTSEKGSKSSNIAKATAQKHVNNHLERSLNEAESTKKRFSFLSLYSYDTSKSSLYSSM | |
| DSKRKPSPPSQRRPKKDDSYQTNSKNHYITASNMQTSHQVSKDLPAPTMVQNKCTLETKKAVRSNRSSI | |
| MVSEVNKASVDNKAAQSPEHSTAKRVLGFFKRRSMKI | |
| C. albicans orf6.4215 | |
| MSTIPSQVEINFNKIHQRSNSSSSTSSYRIPSGNSCIPRTVEMPSLPPTSTHHQHQQMPSSSSHAHIAK | (SEQ ID NO. 8) |
| KIHREVRFGAYILGSTLGEGEFGKVKLGWRKDGKHPSQVAIKLIKRSTITKDSDSEIKIHREINSLKLL | |
| NHPNIVNLVEVMKSGKYIGIVLEYASGGELFDYILQHKYLKENVAKKLFAQLVSGVDYMHAKGLIHRDL | |
| KLENLLLDKHRNVIISDFGFVNSYNRDKNDLMKTSCGSPCYAAPELVLSQTAYEGRKVDIWSLGVILYA | |
| MLAGYLPFDDDPENEDGSDIIKLYHYICKTPLTFPEYVSPLARDLLRKIIVSDPKKRISIDDIRNHPWL | |
| SSHANLLSIRQPEWDKVHSEKQQPIAVEPPQPNKRYSMINERTNSSSLMSPAPRVTHTQPLSSHARSYS | |
| STSISLLYSSPSATPSMANAVTNGEGATTTTTNGSINESNDTLQLSGTPSPKKPSTVSPVRGHQKSASI | |
| SNSYSSASIALKAVVHEENRLHNHQQSQQYIPRSSTISTIVESPTKANTATETETTDGNKILLPPPSKD | |
| AQKLPHAAKKPRPTSYHPSSMSSALIHNNQNPTDVLKMPSPINFPMTQFISTSPPKSNGSLNDSGNFTN | |
| CSPKATSRRNSVVTHVHVNGVLSKENLIHSSSSPDNKRNSVLSYLEDKIDTLELTESHSPSKNTFEEIV | |
| DAAIATPEINQVPVFDSQTSPNGIGLDIKMKEFDETSLVVEKKSKETVSDSKSEESTKETQQQENVVMY | |
| EPIVPVEEYIKKPEEVNSVEAKQPEEVKSEDKSLQGQKSQQQQQQPEKHSADIGKTKVDLKKSASQKKK | |
| VKEESIKKQKDVDSKPIERRHTIAARRHHNDENKENKDVKKRNRFSLLSFYSSYNSSNSNVSLATSKVP | |
| SNSENNTTVLKPTSMNTTRKVLEPSNETNIMRKETKQTNSNGSTTSKSTTSSSSSSAPAASSSSSSSTS | |
| KRASTATKETSAARKVMDFFKRRSVRVG | |
| S. pombe SPAC 140.05 (gi\|7523475) | |
| MNAQPFHNNTSDVQSFQDIISNSYQKPLSLVDSTDRALPDSSLSSLSRSTFQFHKHHLSGNENPQPSSE | (SEQ ID NO. 9) |
| SPYFTNNERLNSSSFPQIHDNQLSPSFNTSYQAIPSSSSNRSRGGPYTPSIRDDSLLALLSFSSNBRLH | |
| SMPSQLQPFNNASSYTTPMAPFTASFSNKVSHSAYPTRRLPSQAKKTSAIERVPVNLNFLQSDNLVVQS | |
| SPQTNFENFEFPKKIPSKEDLETREVLLLPPQTSKLSNKMLDTKSFTDVNKISQQGFVEISSNSSKVTP | |
| NTSLHQSFGIASSSSNNYMQTSSELTSSTEKMNGSHPLQLSNKSLLSIHLMQSKNQGHVSMTGSDKLSS | |
| HVQSETENAPVSKPSKPNTLTEDEKPLQSTKLPGNSLTVGELYQEPKSIQLPELSVSRTTYSAQSSSVK | |
| NCNERIPSAKALKKQKHLVPENKSKLQYVWQKKESLPYANLTSASNTHFFLSENQNDTSERLTRTLRKS | |
| TKNYTFGSYILGRTIGTGEFGKVKLGWPLPKANSTIHRSTPQVVIKIVLSTKQNCQTSRLMREVAILKG | |
| LGNNRPHIVKYLDFVKTKHHFGIVLDYVNGGELFDYILARRRLEDSVACRLFAQLISGVAYLHSRGVVH | |
| RDPYSESY | |

GIN4 Family

The GIN4 fungal-specific kinase family includes three genes from S. cerevisiae GIN4 (SEQ ID NO. 10), HSL1 (SEQ ID NO. 11) and KCC4 (SEQ ID NO. 12); two genes from C. albicans, orf6.4613 (SEQ ID NO. 15) and orf6.6556 (SEQ ID NO. 16); and the CDR2 (SEQ ID NO. 13) and CDR1 (SEQ ID NO. 14) genes from S. pombe.

All three S. cerevisiae members possess similar, and partially redundant, functions from nutrient sensing and developing cell structure to control of cell division. All three genes, GIN4, HSL1 and KCC4, are required during cytokinesis to organize septins within the neck of the bud. The protein products of these genes signal the state of the cytoskeleton to the Swe1 mitotic checkpoint in order to allow mitosis to continue, and also are required for mitotic arrest during nitrogen deprivation.

Cdr1 in S. pombe phosphorylates and negatively regulates the Wee1 mitotic control gene and is involved in mitosis and nutrient sensing.

An inhibitor of one family member would likely inhibit other family members, causing defects in cell cycle or cytokinesis. A GIN4-family inhibitor might also cause fungal cells to ignore stress signals, thereby inducing cell mitosis and proliferation. However, in the absence of nutrients these processes most likely would lead to death of the fungus and its progeny by starvation.

The bold, underlined portion of each family member denotes its kinase domain. Accordingly, the present invention allows the skilled artisan to identify and classify newly-identified kinases from other fungi as belonging to the GIN4 family if the newly-identified kinase domains share at least 55% amino acid sequence identity with any one of the kinase domains of an indicated family member listed below. Preferably, the GIN4 kinase domain has a percentage sequence identity to a kinase domain of any one of SEQ ID NOs. 10-16, of 60%, of 65%, of 70%, of 75%, of 80%, of 85%, of 90%, or of 95%.

| | |
|---|---|
| S. cerevisiae GIN4 | |
| MAINGNSIPAIKDNTIGPWKLGETLGLGSTGKVQLALNGSTGQEAAVKVISKAVFNTGNVSGTSIVGST | (SEQ ID NO. 10) |
| TPDALPYGIEREIIIWCLLNHPNVLRLYDVWETNTDLYLVLEYAEKGELFNLLVERGPLPEHEAIRFFR | |
| QIIIGVSYCHALGIVHRDLKPENLLLDHKYNIKIADFGMAALETEGKLLETSCGSPHYAAPEIVSGIPY | |
| QGFASDVWSCGVILFALLTGRLPFDEEDGNIRTLLLKVQKGEFEMPSDDEISREAQDLIRKILTVDPER | |
| RIKTRDILKHPLLQKYPSIRDSKSIRGLPREDTYLTPLSESNSSIDATILQWLVILWHGRDPEGIKEKL | |
| REPGANAEKTLYALLYRFKCDTQKELIKQQQVKKRQSISSVSVSPSKKVSTTPQRRRNRESLISVTSSR | |
| KKPISFNKFTASSASSSNLTTPGSSKRLSKNFSSKKKLSTIVNQSSPTPASRNKRASVINVEKNQKRAS | |
| IFSTTKKNKRSSRSIKRMSLIPSMKRESVTTKLMSTYAKLAEDDDWEYIEKETKRTSSNFATLIDEIFE | |
| YEKYEQIRKEKEELERKVREAKAREELERRRRKQEEKERARKLLEKEDLKRKQEELKKQIEIDISDLEQ | |
| ELSKHKEEKLDGNIRSISAPMENEEKNINHLEVDIDNILRRRNFSLQTRPVSRLDPGIMFSSPTEEVSP | |
| VEPKRTENERLTTEKKILETIRRSKFLGSSFNIDKELKLSKMEYPSIIAPQRLSEERVVSDSNDGYESL | |
| ILPKDGNGVSQLKDSTATTAPVSDGRLRKISEIRVPQFTRKSRHFSESNKRLSVLSMYSTKESFTNLVD | |
| ILKNGNLDVNNQQSQRIPTPRSADDSEFLFETVNEEAEYTGNSSNDERLYDVGDSTIKDKSALKLNFAD | |
| RFNGSNEAKQTDNLHLPILPPLNGDNELRKQNSQEGDQAHPKIKSMIPESGSSSHTEKEEENEEKEEKK | |
| PEQHKQEEDQEKREKVVDDMEPPLNKSVQKIREKNAGSQAKDHSKDHLKEHKQDKNTAIGNGSFFRKFS | |
| KSSDKTMELYAKISAKQLFNGLEKLLRGWTQYGLKNIKSHPNNLTLTGKLSSDNIFSLRSTLFEVNIYP | |
| RGKMSVVQFKKVSGSFKAVKKLNEVENVLNKEGVLQK | |
| S. cerevisiae HSL1 | |
| MTGHVSKTSHVPKGRPSSLAKKAAKRAMAKVNSNPKRASGHLERVVQSVNDATKRLSQPDSTVSVATKS | (SEQ ID NO. 11) |
| SKRKSRDTVGPWKLGKTLGKGSSGRVRLAKNMETGQLAAIKIVPKKKAFVHCSNNGTVPNSYSSSMVTS | |
| NVSSPSIASREHSNHSQTNPYGIEREIVIMKLISHTNVMALFEVWENKSELYLVLEKVDGGELFDYLVS | |
| KGKLPEREAIHYFKQIVEGVSYCHSFNICHRDLKPENLLLDKKNRRIKIADFGMAALELPNKLLKTSCG | |
| SPHYASPEIVMGRPYHGGPSDVWSCGIVLFALLTGHLPFNDDNIKKLLLKVQSGKYQMPSNLSSEARDL | |
| ISKILVIDPEKRITTQEILKHPLIKKYDDLPVNKVLRKKMRKNMARGKSNSDLHLLNNVSPSIVTLHSK | |
| GEIDESILRSLQILWHGVSRELITAKLLQKPMSEEKLFYSLLLQYKQRHSISLSSSSENKKSATESSVN | |
| EPRIEYASKTANNTGLRSENNDVKTLHSLEIHSEDTSTVNQNNAITGVNTEINAPVLAQKSQFSINTLS | |
| QPESDKAEAEAVTLPPAIPIFNASSSRIFRNSYTSISSRSRRSLRLSNSRLSLSASTSRETVHDNEMPL | |
| PQLPKSPSRYSLSRRAIHASPSTKSIHKSLSRKNIAATVAARRTLQISISASKRSLYSLQSKRSLNLND | |
| LLVFDDPLPSKKPASENVNKSEPHSLESDSDFEILCDQILFGNALDRILEEEEDNEKERDTQRQRQNDT | |
| KSSADTFTISGVSTNKENEGPEYPTKIEKMQFNMSYKPSENMSGLSSFPIFEKENTLSSSYLEEQKPKR | |
| AALSDITNSFNKMNKQEGMRIEKKIQREQLQKKNDRPSPLKPIQHQELRVNSLPNDQGKPSLSLDPRRN | |
| ISQPVNSKVESLLQGLKFKKEPASHWTHERGSLFMSEHVEDEKPVKASDVSIESSYVPLTTVATSSRDP | |
| SVLAESSTIQKPMLSLPSSFLNTSMTFKNLSQILADDGDDKHLSVPQNQSRSVAMSHPLRKQSAKTSLT | |
| PRSNLNANLSVKRNQGSPGSYLSNDLDGISDMTFAMEIPTNTFTAQAIQLMNNDTDNNKINTSPKASSF | |
| TKEKVIKSAAYISKEKEPDNSDTNYIPDYTIPNTYDEKAINIFEDAPSDEGSLNTSSSESDSRASVHRK | |
| AVSIDTMATTNVLTPATNVRVSLYWNNNSSGIPRETTEEILSKLRLSPENPSNTHMQKRFSSTRGSRDS | |
| NALGISQSLQSMFKDLEEDQDGHTSQPLILESSMSYSKRRPSEESVNPKQRVTMLFDEEEEESKKVGGG | |
| KIKEEHTKLDNKISEESSQLVLPVVEKKENANNTENNYSKIPKPSTIKVTKDTAMESNTQTHTKKPILK | |
| SVQNVEVEEAPSSDKKNWFVKLFQNFSSHNNATKASKNHVTNISFDDAHMLTLNEFNKNSIDYQLKNLD | |
| HKFGRKVVEYDCKFVKGNFKFKIKITSTPNASSVITVKKRSKHSNTSSNKAFEKFNDDVERVIRNAGRS | |
| S. cerevisiae KCC4 | |
| MTVANTETHSAAKPSSTIGPWKLGETLGFGSTGKVQLAQHERTGHRTAVKVISKSIFNNNGNHSNDDSV | (SEQ ID NO. 12) |
| LPYNIEREIVIMKLLSHPNVLSLYDVWETNNNLYLILEYAEKGELFNLLVDHGPLPEREAINCFRQIII | |
| GISYCHALGIVHRDLKPENLLLDSFYNIKIADFGMAALQTDADLLETSCGSPHYAAPEIVSGLPYEGFA | |
| SDVWSCGVILFALLTGRLPFDEENGNVRDLLLKVQKGQPEMPNDTEISRDAQDLIGKILVVDPRQRIKI | |
| RDILSHPLLKKYQTIKDSKSIKDLPRENTYLYPLADSNNHTSASIDDSILQNLVVLWHGRHADDIVSKL | |
| KENGTNKEKILYALLYRFKLDSVRGSNKKNRNKIKKTKKNKRSSTLSSSSSLLLNNRSIQSTPRRRTSK | |
| RHSREFSSSRKRSSFLLSSNPTDSSPIPLRSSKRITHINVASANTQATPSGVPNPHKPNSKKRSSKRLS | |
| YMPNTKRSSLTSKSLSNFTNLIDDDDWEYIEKDAKRTSSNFATLIDEIFEPEKFELAKREKAELQRKVQ | |
| EAKRQSVNAQKINEDEFGSEVSDGMKELKKINDKVSSPLINYEFSQQELLQDIDTLLTNRYQLSSYTRP | |
| ISRLDPGLTPVTETLPNNLKEKTALLQDTEKKTIETIRRSKFLGSLLNVRGGLSPGKSELAPIEESPIV | |
| STTPLIYNDRMEPRRISDVEVPHFTRKSKHFTTANNRRSVLSLYAKDSIKDLNEFLIKEDPDLPPQGST | |
| DNESRSEDPEIAESITDSRNIQYDEDDSKDGDNVNNDNILSDFPQGVGISQEYDMKDKNPNQSPISKSA | |
| EPTLVVKLPSLSSFQGKNASGLGLYQREPSKVTLPSLTSNNSSVGENIEDGAEKGTESEKIAASLSDDD | |
| LKEDNDKKDNDTVNAPTTVKKPPNSVLLKKFSKGKILELEIHAKIPEKRLYEGLHKLLEGWKQYGLKNL | |
| VFNITNMIITGKLVNDSILFLRSTLFEIMVLPNGDGRSLIKFNKKTGSTKTLTKLATEIQIILQKEGVL | |
| DK | |
| S. pombe CDR2 (gi\|2058369) | |
| MSTISEVGPWELGLSLGSGGPNSSRLAKHRETGQLAVVKPIVGWSELTSSQQARIEGELVLLRLIEHPN | (SEQ ID NO. 13) |
| VLQLIDVISAQEQLFVVVEYMPGGELFDCMLRKGSFTEQDTAKFLWQILCGLEYCHKLHICHRDLKPEN | |
| LYLDAHGSIKIGEFGNASIQQPGKLLQTSCGSPHYASPEIIMGRSYDGCASDIWSCGIIFFALLTGKLP | |
| FDDDNIRSLLLKVCQGQFEMPSNISPQAQHLLYRMLDVDSSTRITMEQIREHPFLSCFVHPNISIPIIS | |
| APIQPIDPLIVQHLSLVFRCSDDPMPLYEKLASQSPLVEKTLYTLLSRHLHPPSSAAVDRNRAVVDDLL | |
| GTAASNGQQMDEEEIEQAINIPTLAPYPISYAAESVPRPATSASPFLTPVTTSGTFNYSFNATNPQSIL | |
| QRPATTSSAVPQLPKSVTPGLAYPHDSSMLSSNYRPPSALSPRNFNVSINDPEVQLSRRATSLDMSNDF | |
| RMNENDPSIVGNLAASNFPTGMGPPRKRVTSRMSEHTGNRVVSFPRGSAFNPRVTRFNVGNEQFSNNID | |
| NNNYNQPYANATMNNSRRLRTPSGERSMRADLSQSPASYDSLNVPKHRRRQSLFSPSSTKKKLSGSPFQ | |
| PKRSFLRRLFSSEPSCKCVYASLVASELEHEILEVLRRWQLLGIGIADIIYDSVSASISARIKRQNSLN | |
| LKPVRFRISVLAEFFGSQAVFVLESGSSTTFDHLATEFQLIFEDKGFLDNLELSYFQASASRPVSRMSV | |
| SSSPFAVFRQRQSVQS | |
| S. pombe CDR1 (gi\|7708585) | |
| MVKRHKNTIGVWRLGKTLGTGSTSCVRLAKHAKTGDLAAIKIIPIRYASIGMEILMMRLLRHPNILRLY | (SEQ ID NO. 14) |
| DVWTDHQHNYLALEYVPDGELFHYIRKHGPLSEREAAHYLSQILDAVAHCHRFRFRHRDLKLENILIKV | |
| NEQQIKIADFGMATVEPNDSCLENYCGSLHYLAPEIVSHKPYRGAPADVWSCGVILYSLLSNKLPFGGQ | |
| NTDVIYNKIRHGAYDLPSSISSAAQDLLHRHLDVNPSTRITIPEVFSHPFLMGCTSLSSMDSTTPPTPS | |
| LSIDEIDPLVVDCMCVLWKKSSSKKVVRRLQQRDDNDEKYVYKVLSEILRDDMLKKQRFDENKYLSLYD | |
| LIHDNNLFTKASISTTSLVKSNVSTNSRKSSNFEDELARRVSSPLSALNQMSQSPIPIRVSSDKDYDSY | |
| ACHEVVSNPSTLDDDYNYMFVCPPEEYTYSTDNVRTDSLDLQSLPTPTLEQLESVPFNRYGYVRIFPST | |
| TLSSTASGYYTPDSLSTPEPSIDGLTNLDDVQVGGFVQGSGNQNRRPISFPVISNMQPNITNVRSASAP | |
| LCSSPVPSRRYSQYATNARYTPRKVSSGSVLRKISSFFRKD | |
| C. albicans ORF6.4613 | |
| MSTVVNRRSSHQFDSPSNHLDHSSSMNVDKVVQSVTNATKRLSQISTNTNNSNKKRKTQNKIGPWKLGR | (SEQ ID NO. 15) |
| TLGRGSTGRVRLAKNTTTGQLAAVKIVPKSNFKKLENPKYKRSKEDATRLPYGIEREIIINKLISHPNI | |
| MGLYDVWENKNDLYLILEYIEGGELFDYLIKRGKLQEYEAINYFKQIINGINYLHQFNICHRDLKPENL | |
| LLDFNKNIKIADFGMAALEVKEKLLETSCGSPHYASPEIVAGKNYHGAPSDIWSCGIILFALLTGHLPF | |
| DDENIRKLLLKVQSGKFNMPPELSFEAKDLITKHLKVNPRERITIDAILTHPLLAKYPEPTVSYSSTTT | |
| LDINSINIKQIESVDKIDKEILKNLSVLFHNCDEKTIISRLLSPNRCPEKMFYYLLMKYRNEHLSNSNS | |
| FNSSNDVDSARSLPRSTSYVKTTVTDHATGEKHTTVKKIQQSSSIYSNRSLLKKSTSAKGNVLSNITNR | |
| PNTPKQFSASSSFNKKKALHSKTQIYASRSRNASSRSLKSNSSTGRNGNNASVTSVNKIPEITGATVLQ | |
| PIPSMAMNRGDEQQNKTKKNLTGTFGNKSLLNFQLICEEVFENDKENSKPVSKTPVSQLPPPPPPPIET | |
| PTSRTNSVKRGKTWSLARRERELAEQVRQRNEARENKLKAEELARKELEQEKKRIAEEKKRLEQQEREL | |
| DEKQKLQEKQKAALEKLQKHQSAHDFEGLFASNRRSVTDMAPSSGMSSLDPRAHMVSRANTIGSPNLSS | |
| SSVNIDENASKVLHKFGIDVAPSPKRFSRASKTSTSKNLSSFLAPTVSRNLSSQLKTSSSKNLAGYLHG | |
| TTDTNGSAIAAKKKDDSTNEALTIEEFNAKERTSMSPSISKASVNKPNSNQSSYYRSMFSDNGNDDNVT | |
| KVRTGESHLSVQEEEEMDMENAIDEDISLIPNPRFSRFSFGGLLGSNTVANEEGDWTIMNSTLNHSNTV | |
| VRGTHNKSSTMLGLGIKMRDTTTIKEDEEFEDEKPFISVPSSEDDEGNTHKNKRGGLRDSGNYDFDEEH | |
| SVASTANTEYSDVASQGQQMPGSHTIHQLETELSNFDLLSYRVADIGKVNKHKFSIVDSKETLLKNHSS | |
| DEATIEVKEDNNEHDFNDKIKQHYDDNGDSEEDDEDEDEEEEDDDDDDDARSSFEARPHSHNYSLAEIT | |
| SESPVGGGYESPSIANDFKKSRHSTGIFSTTQFPRSPYVVNNNGDSNKDENSQQQTKHMLNDGHKGLIT | |
| SPVQDTFGSKKPVESNSLFRRLSLNPNRAAPKAPAPPPPSAPISSAAKANISQPLSSPTKGHNRFSRIS | |
| IGSKNMLQKEDKSTKSNWFKKFFHSLTTPSAKDQSGNSSSKVASKDIKIIDTSLTAAQLIRVIKYQLEL | |
| KKIEGSISKVDIDEEFGLISGVIPSKFANGRKLKFKIEVIDLINSSSLHVIKMKGNDKGFQSLVNIVTF | |
| IIKKEEQDKISRR | |
| C. albicans ORF6.6556 | |
| MPHSRQPSISSSIMSQSNHNHPQKIGPWKLGKTLGRGATGRVLLATHQTTGQKAAVKVVSKSELQDEET | (SEQ ID NO. 16) |
| EKNGDGLPYGIEREIIIMKLLTHPNVLRLYDVWETSKALYLVLEYVEGGELFDLLVERGPLPEVEAIKY | |
| FRQIILGTAYCHALGICHRDLKPENLLLDSQLNVKLADFGHAALESNGKLLETSCGSPHYAAPEIVSGL | |
| KYHGAASDVWSCGVILFALLTGRLPFDDENIRNLLLKVQAGNFEMPVDEVSREARDLIARMLEVDPMRR | |
| ISTEKILRHPLLTKYPMSNEDLISEKSLPHPQTGYKSLGSVRNIDKQILSNLTILWNDRPEEEIVDCLL | |
| KDGSNPEKTFYALLMRYKHNQEDNTNNNSPKKSTSFNNKVVRSGSKYSLNGTPRRKPASHISVSRPTSF | |
| QYKSNPGAGATANRNSVARHSVASSANNSPRKSPYKSPYRSPYRSPYKSPSKRYSYNQSPTKSPYGRRS | |
| NSQRQFENEPLKAKPRNIYNEIVDAQSNFSLPPSLPPSLPSKDSRYMIDEPNQPQLQQPALSQVPENPI | |
| VDESPDLMQSAKISSGKRNSIIGKNNNNSNSNKRMSKRKSIRASMTTGLKRNSITMKLLSTYAKLSGDD | |
| DWEYMDKQTKRTSATFAALCDKIFNQEDYDEEDEQLVDPEEKEAKEYERLMELERKKHEAELKARRELE | |
| KKKRRQKRRSILSSKKLSIIVKNDADPNNSEQELVDEGIKQPKRQSKNLTALRALSEGNHASEELTLED | |
| VENLKRRSASQPVPKRRQTPVLTRRPVSRLDPLWQAHENEQLDRAKDALEQEWRDSQKRSSTVSRKKVN | |
| RESMISVMDDIVEEDQGRVNRRSTRNTYYERERDYELPEPTVEDSNLTDDYMTEIRKSRLLNSQLNVRD | |
| PLNEKRKSEPKTLISNVQIPSVTRKSRNFTTSNKRLSVLSMYSTKESYRDLNSIINSPDENPEQHQNMN | |
| KPALRTSIADRLDKAGLAEPEYETETDGEDKVSVIDLDDHLADRRTSYYDGSGKRASRASTTKRYNVHS | |
| SSGQRPKSKVPDLPKNDYDDTFVSNSDEVHKRQYKSMVSDESSASDDVFDKIKLPDGKSTKSSIDELAN | |
| GTSTSGHRKPKIRHSQPGPEMLIPHLNGGIESSQPMSKVRGNNSSGHDDSVPPPPPAHKVNKKPLDDKT | |
| NFPPPEVDPKRKGSFFRKLSWGSKKTIENNTNAATNTTTQQQLPSPAESKEEKPKSSFFRWFSSSNTPS | |
| AAEIRKFNTILPKHEMSTALFALLNSWSNFGLKDLRNDQVGYYITGAISKHNSFMLKSCKFRIKINQRD | |
| FNQKSEIVCVRVKGSKVTTDTLFSEIEKVLLKEGVLDK | |

RAN Family

The RAN fungal-specific kinase family includes three genes from S. cerevisiae KSP1 (SEQ ID NO.17), SKS1 (SEQ ID NO.18) and YDR247W (SEQ ID NO.19); the RAN1 (SEQ ID NO. 20) and SPBC16E9.13 (SEQ ID NO. 21) genes from S. pombe, and un-named homologs from Pichia jadinii (gi|1232133) (SEQ ID NO. 22), Nectria haematococca (gi|11256839) (SEQ ID NO. 23) and C. albicans (gi|17271026) (SEQ ID NO. 24).

The RAN kinase family members are implicated in a variety of functions. KSP1, for example, suppresses mutants in SRM1, a GDP exchange factor that is involved in splicing and nuclear export, when it is overexpressed. The SKS1 kinase interacts with a proteasome subunit involved in transcription as well as with telomeres. It may also be used by fungal cells during carbohydrate metabolism in times of nutritional stress. RAN1 regulates mitosis. Furthermore, RNAi interference experiments reveal that the C. albicans' kinase, depicted in SEQ ID NO. 24, is required for normal growth.

The bold, underlined portion of each family member denotes its kinase domain. Accordingly, the present invention allows the skilled artisan to identify and classify newly-identified kinases from other fungi as belonging to the RAN family if the newly-identified kinase domains share at least 55% amino acid sequence identity with any one of the kinase domains of an indicated family member listed below. Preferably, the RAN kinase domain has a percentage sequence identity to a kinase domain of any one of SEQ ID NOs. 17-24, of 60%, of 65%, of 70%, of 75%, of 80%, of 85%, of 90%, or of 95%.

| | |
|---|---|
| S. cerevisiae KSP1 | |
| MTLDYEIYKEGGILNNRYQKIEDISEGSYGYVSLAKDVREKRLVAVKYIFKLEDDGQYDGPQDDENDCD | (SEQ ID NO. 17) |
| SSDCDDDEDTKVDTDRHENENGNASSNNGSSREKKHNLYKHKKSLISSKVKSRLSNNICLEAMYEVDIQ | |
| TKIGRHQNIAALLDFFDSYIIMEYCSGGDLYEAIKADAVPKKTKSITHIITQIMDAIEKVHNKGIYHRD | |
| IKPENILISGIDWTIKLTDWGLATTDKTSMDRNVGSERYHSPELFDSNLDIKERKEPYDCAKVDLWAMG | |
| IVFLNIVFHKNPFSIANQSDKSFCYFAANREALFDVFSTMAYDFFQVLRYSLTIDPANRDLKMKRTELQ | |
| NLSEYTLDDEYYNNLDEGYEETMIDGLPPQPVPPSSAPVSLPTPISSSNKQHMPEFKKDFNFNNVNERK | |
| RSDVSQNQNVASGFFKKPSTQQQKFFNQGYNTTLSTHEPAKSAPKFKFKKRNKYGRTDNQFSKPVNIED | |
| RKKSKILKKSRKPLGIPTPNTHMNNFFHDYKARDEFNTRDFFTPPSVQHRYMEGFSNNNNKQYRQNRNY | |
| NNNNNNSNNNHGSNYNNFNNGNSYIKGWWKMFNKYRRPSSSSYTGKSPLSRYNMSYNHNNNSSINGYAR | |
| RGSTTTVQHSPGAYIPPNARNHHVSPTNQFLRVPQSTAPDISTVLGGKPSYQEHYTQDSMDSEGDHDSD | |
| DVLFTLEEGDHDFVNGMDNLSINDHLPHTTVGSHNEVFVHASTNHNNNGNNNHIDTNSTTNQYHRQYTP | |
| PPLTTSLHINNNNNESNELPDLLKSPASSEAHLNLSSGPIDPILTGNIGNRYSHSSDSKELEQERRLSM | |
| EQKFKNGVYVPPHHRKSFNLGTQVPPMNMKTSNEATLSVSHNSVNFGGSYNSRRSSANESNPLHMNKAL | |
| EKLSSSPGAKSSFVGFPKPLLPRNHSSTTIALQNEDVFADSNNDAIIFEDEEYEGESDKMAHGKMEGGD | |
| NESSSTSPDERQIFGPYEIYAQTFAGSTHDKKLGAGRKTSIQDEMVGSLEQYKNNWLILQQQD | |
| S. cerevisiae SKS1 | |
| MLSDCLLNNFRITAQIGSGAYGLVFHVVDILTSREYAVKTVFKSSSMDEFYNKNGLNNNSQVARTTLLQ | (SEQ ID NO. 18) |
| TQLYHFFKSFQKKLFLPSVDLDSILQLTENELNRLPHYREIAFQLRVQSHGNIVKIHQVLESSIATFIV | |
| HDYYDRDLFTSIVDDKHFVNHGILIKKVFLQLCSALDHCHRLGIYHCDIKPENVLLDRNDNAYLCDFGL | |
| STKSKYLAPNVCVGSSYYMkPERILYCLNTTTNGIHVDECCSSLPTDTGDIWSLGIILINLTCIRNPWL | |
| KAHQKEDNTFQHFANDNNVLKKILPISDELFTVLTKILQLNPYTRIDMKTLMSEVSSLTSFTREGPLSQ | |
| VPILSSEVYMTHIIRNENLFLSDLSHFSADQEQQQQQQQQQQQVQEQEQEQKQEQIQNQEQAQQQQEEE | |
| DAEPESDIPSTYNSDGSMEKYEYTNNHNNSTFLTSSMDSTPYQSDIDDVSASKDCKFQQDTLRNRLLCL | |
| QMNFSTLTDGPNEKWLPDY | |
| S. cerevisiae YDR247W | |
| MMMFHNCRINYLITSQIGEGAYGLVYRALDIRTDRQYAIKAVVQSYGVSKEADMGNDKIHKNSVKLQIC | (SEQ ID NO. 19) |
| KLAKLFKESKNVVRVPSIDLESIENMSEEDFKKLPHYKEISLHLRVHHHKNIVTIHEVLQSAVCTFIVM | |
| DYYPTDLFTSIVDNRHFVTNGLLVKKVFLQICSALNYCHEHGIYHCDIKPENLLLDTEDNVFLCDFGLS | |
| TTSTYIKPNVCIGSSYYMPPERISFDGRVSSSKSGGHKLGKVCPSCNGDLWSLGIILINLTCIRNPWLK | |
| ADKTEDNTYYYFTKDPNILKQILPLSDDFYSLLSKILQVNPKNRMSLQELMKEVSSITSFTNEGPLSKV | |
| PPLSKSVYEKFVSPVDNTNENLSPKSYVYMHDSKAAKNLSYTSSSEEEDGIKEGIDDDNGSRSGSFGTL | |
| DTDTGLHSSFTSTSCESDNECSKISNKFSLFEKKFNELRMSSSSLTN | |
| S.pombe RAN1 (gi\|5689987) | |
| MMRENPELLLGQVLGDSLRFVSIIGAGAYGVVYKAEDIYDGTLYAVKALCKDGLNEKQKKLQARELALH | (SEQ ID NO. 20) |
| ARVSSHPYIITLHRVLETEDAIYVVLQYCPNGDLFTYITEKKVYQGNSHLIKTVFLQLISAVEHCHSVG | |
| IYHRDLKPENIMVGNDVNTVYLADFGLATTEPYSSDPGCGSLFYMSPECQREVKKLSSLSDMLPVTPEP | |
| IESQSSSFATAPNDVWALGIILINLCCKRNPWKRACSQTDGTYRSYVHNPSTLLSILPISRELNSLLNR | |
| IFDRNPKTRITLPELSTLVSNCKNLTRRLRPAPLVSSRYLAYQQQQQQQQMNLQQGIQGYPHQGYMPTQ | |
| NIGFPWPPTPQFVSNWNBCATPTIPVSLQVLTPNSSLKVDPTTPLTAPIHATESFWPSAAAAAAAVHNN | |
| ANSYMPITPTPYPNNAKIFGYPNQPPLTPIPFTGFVLHPAPVGRAADAVDPSRKSL | |
| S.pombe SPBC16E9.13 (gi\|19112587) | |
| MKLLQKKGYKVERPLNKGSYGTVVLAHRLFRTPRCKDLKYAIKCIKKPAYTFLQEVNILRQLSRSRHRN | (SEQ ID NO. 21) |
| IIHFVESFEDNVYYYVVLEYCPLGDLYECILNNDFPNAENQPEMIKNIFLQIIDGVAHLHSHGIYHRDL | |
| KPENFLLSLSEDGSELVVKISDFGLACRDKISYDFGTGSDRYMAPEQFEEVDGAGYSPRAADIWALGIC | |
| LLNLIFARNPFTYPHEKDPIFADYMLDAMTLFDVFPTLSQDTYNVLRACLCVSPEKRSLAKTREAVLAV | |
| TKWTTDDEELESFVNEEEEFRASDFMPAEDNVRCTQSDREPLRTPSVLTPANTIQRGLLPSKLPALSDV | |
| DENISTSSSPRSPASLAPVNNSERSYDSGLGESLNNMHIGKSIATAVPVNTKRSPYSCSAPAIVFPNSI | |
| KGNKDHLKFGRSWCDMDEEDEEDIVSFGSNDDFGASDELSSKHIGLADDWNVLSQWNDNS | |
| Pichia jadinii (gi\|1232133) | |
| MTHTDITGSLINEYRIVKLIGSGAYGLVYQAQNTVTGQQVAIKCISKKSNPSVKKQSDYLTTLLAEHLL | (SEQ ID NO. 22) |
| ERDFSLQGLREMSLKRLSMADNIPCPFVREISIHLQVHQHPNVISIHKILDSQVAVFVVMDYYPEGDLF | |
| VNIVDRQVYARSSGLIKDVFIQLIDVISYCHSKGIYHCDIKPENINCANKGSKVVIGDFGLAVKSKYIQ | |
| SKTCIGSSYYMAPERLCTMNHSLTRLEYPACKQDIWSLGVILINFCCTRNPWMKACEKDATYSAFKKDP | |
| KILMEILDISEELWNILCDCFREEPEERISLFELRDRVLKCRSFTVAGPLSRCDSYEQDMDDALECAVP | |
| ANESSVGSNGSLDLPMDHIIEYAQYLQTLSSVKNTAAGNYTQNQFVLDNNIMDNVSIMSNKSFNMNFA | |
| Nectria haematococca (gi\|1256839) | |
| MQHHAIFGYQTPPASPGFDNPKCTIQQPFAVPRHYPTRPLAPEERLGRVLEGTLQLTEILGTGAYGVVY | (SEQ ID NO. 23) |
| LAVDLKTGGKYAVKCLSKFNADGTQLEPRQFAYQQREIRLHWKASNHANVVQMLKIVNDPDCIYVILEY | |
| CPEGDLFLNITERGQYVGKDELSRNIFLQILDAVEHCHNLGIYHRDLKPENILVTDRGDTVKLADFGLA | |
| TSDDRSEDYGCGSTFYMSPECLDPSARKPYYMCAPNDVWSLGVILVNLTCGRNPWKQASFQDSTYRAYA | |
| GSKDFLKTILPLSDELNEILGRIFEPNPEQRITLNELRTRIMACSRFTMPAVSPPTPPASPDHTTQYVS | |
| TEDAIIDDYDYDSPLSPASSSDDEGSLTSSGSTIDDLDDDFDQERQMPQTPPEYAPHAFDPEEPKEHQL | |
| IYHSQEFVPQKYSGPVPVPVQVPVGVPPQPMLCQPVPPVIQAPVPIQAPCQQHKSYFPIWDMVKYVQHV | |
| PILQHHIPFHQQVPFMPTFQGCY | |
| C. albicans (gi\|7271026) | |
| DLCYANSIIDYNELHLVLIDFGLAMDSATICCNSCRGSSFYMAPERTTNYNTHRLINQLIDMNQYESIE | (SEQ ID NO. 24) |
| INGTTVTKSNCKYLPTLAGDIWSLGVLFINITCSRNPWPIASFDNNQNNEVFKNYMLNNNKAVLSKILP | |
| ISSQFNRLLDRIFKLNPNDRIDLPTLYKEVIRCDFFKDDHYYYAQHQHHHNHNQINNAYNHYQKQPNQA | |
| RPTANQQLYTPPETTTYNSYASDMEEDEISDDEFYSDEEDEDIEDYEEEEEEYFGNEQQQQQQVTTVNG | |
| NFGQVKGTCYYDTKTKTTTYIKPPAAYTLETPSQSVEYC | |

ELM Family

This family includes ELM1 (SEQ ID NO. 25), PAK1 (SEQ ID NO. 26) and TOS3 (SEQ ID NO. 27) from S. cerevisiae; SSP1 (SEQ ID NO. 28) of S. pombe; and orf.7535 (SEQ ID NO. 29) from C. albicans.

SSP1 mutants show defects in cell cycle, cell morphology and osmotic stress response. The enzyme is also known to modulate the actin cytoskeleton. Overexpression of PAK1 reveals an interaction with DNA polymerase and hence with cell cycle or DNA damage repair. PAK1 interaction with Tid3, a member of a centromere protein complex, may indicate a further role in cell cycle. ELM1 is required for the cytoskeletal changes underlying cytokinesis, bud development and pseudohyphal growth in S. cerevisiae. Like members of the GIN4 family, ELM1 is required for proper septin localisation.

The bold, underlined portion of each family member denotes its kinase domain. Accordingly, the present invention allows the skilled artisan to identify and classify newly-identified kinases from other fungi as belonging to the ELM family if the newly-identified kinase domains share at least 38% amino acid sequence identity with any one of the kinase domains of an indicated family member listed below. Preferably, the ELM kinase domain has a percentage sequence identity to a kinase domain of any one of SEQ ID NOs. 25-29, of 40%, of 45%, of 50%, of 55%, of 60%, of 65%, of 70%, of 75%, of 80%, of 85%, of 90%, or of 95%.

| | |
|---|---|
| S. cerevisiae ELM1 | |
| MSPRQLIPTLIPEWAPLSQQSCIREDELDSPPITPTSQTSSFGSSFSQQKPTYSTIIGENIHTILDEIR | (SEQ ID NO. 25) |
| PYVKKITVSDQDKKTINQYTLGVSAGSGQFGYVRKAYSSTLGKVVAVKIIPKKPWNAQQYSVNQVMRQI | |
| QLWKSKGKITTNMSGNEAMRLMNIEKCRWEIFAASRLRNNVHIVRLIECLDSPFSESIWIVTNWCSLGE | |
| LQWKRDDDEDILPQWKKIVISNCSVSTFAKKILEDMTKGLEYLHSQGCIHRDIKPSNILLDEEEKVAKL | |
| SDFGSCIFTPQSLPFSDANFEDCFQRELNKIVGTPAFIAPELCHLGNSKRDFVTDGFKLDIWSLGVTLY | |
| CLLYNELPFFGENEFETYHKIIEVSLSSKINGNTLNDLVIKRLLEKDVTLRISIQDLVKVLSRDQPIDS | |
| RNHSQISSSSVNPVRNEGPVRRFFGRLLTKKGKKKTSGKGKDKVLVSATSKVTPSIHIDEEPDKECFST | |
| TVLRSSPDSSDYCSSLGEEAIQVTDFLDTFCRSNESLPNLTVNNDKQNSDMKTDRSESSSHSSLKIPTP | |
| IKAMIRLKSSPKENGNRTHINCSQDKPSSPLMDRTVGKRTVNNSGARKLAHSSNILNFKAYINSEDSDI | |
| RETVEDVKTYLNFADNGQI | |
| S. cerevisiae PAK1 | |
| MDRSDKKVNVEEVNVPSNLQIELEKSGTSSSVSLRSPTKSSATNLAGMAEGARDNASIASSSVDSLNML | (SEQ ID NO. 26) |
| LERQRVRQLNHPQHQQHISSSLAKTPTTTSSFCSSGSSKNKVKETNRISLTYDPVSKRKVLNTYEIIKE | |
| LGHGQHGKVKLARDILSKQLVAIKIVDRHEKKQRKFFTFIKSSKISENDKIKREIAIMKKCHHKHVVQL | |
| IEVLDDLKSRKIYLVLEYCSRGEVKWCPPDCMESDAKGPSLLSFQETREILRGVVLGLEYLHYQGIIHR | |
| DIKPANLLISGDGTVKISDFGVSLAASSTNSSDSSESLDELELAKTVGTPAFPAPEMCLGEDAFTRYNL | |
| TKENLFRGSCISFMIDIWAVGVTLYCLLFGMLPFFSDFELKLFEKIVNDPLKFPTFKEIQSNKVSKVSC | |
| EEEYEMAKDLLLKLLEKNPQKRMTIPAIKKHPFVSWDFDHVPENDEKLLSSVLEQKLRFQCNQTDQFEP | |
| ISISKHELKNAVSGVGKKIKESVLKSIPLKDPSDLSNKNYLHPTETTRGRGDANVIVSEGSVLSNIKEL | |
| SANDGCLNTDSDTNININDDDHYSGDDNDGHLTKRELERELNKFDDKHEAGNMVNLPINSSFASLDSFY | |
| IDNFAMARMGMSSPEAGDSVSSVPNLPSAPSSTRLGRSPVFSGVTNQPSPIRPVLPQQKSSFCATGRYD | |
| KSHNSLLRNSSSHLTSYNSGRPSSRTGRMNSRNQNLPKIPNSLSKISTTKLTELRVPKDSEIPSPAKNP | |
| NADRLRRFPVKKNTKTPAIKDPPRININSSDKSGSKNSPIKSLYQRMKQSKDNSKTFEVRRGNFFSHFN | |
| GDDDDSSSQSSVTSSGSESDSELSSTSSSCTSGTQSRNSSNNNAYSETESLPFEFGVDSEDGSGVLLRD | |
| LPNEDQIRPFLDIQPCRRMKVKSSLNLEPPSVSSSSSSSSDEDELILNVGTAGHRRRHNSSKLSELSNS | |
| PQKGSNNFMYSNGSVHDSETTITPQNMDDLTLHQALSRSQPISKPGPLVLPKRLDQKKATTETSNLTDI | |
| VEFNGNNDHRKDKNFDKVLYSRDLLKLALSSTNAGRRRSIPSNKIRGRKDASITMSTNVGNDEHARNTS | |
| CHGDKGQENGAIKQRTHERSRSLTVAELNEEKRRSALP | |
| S. cerevisiae TOS3 | |
| MVLLKEPVQPLPRSSLLYNNASNSSSRIKETRKVKLLYNPLTKRQILNNFEILATLGNGQYGKVKLARD | (SEQ ID NO. 27) |
| LGTGALVAIKILNRFEKRSGYSLQLKVENPRVNQEIEVMKRCHHENVVELYEILNDPESTKVYLVLEYC | |
| SRGPVKWCPENKMEIKAVGPSILTFQQSRKVVLDVVSGLEYLHSQGITHRDIKPSNLLISSNGTVKISD | |
| FGVAMSTATGSTNIQSSHEQLLKSRALGTPAFFAPELCSTEKEYSCSTHEIWSLGVTIYCLLFGKLPFN | |
| ANSGLELFDSIINKPLEFPSYEEMLNGATSGITMEEYTDAKDLLKKLLQKDPDKRIKLADIKVHPFMCH | |
| YGKSDAASVLTNLETFHELKVSPPSSCKRVELVSLPVNSSFASLDSVYMENFDHNNLRTGADRNSTYSP | |
| SIYDANTLSPSAYHNIGSRESSYSSFSSFTSSTAFASQISIQDAPAIGDQQCLIGESGSSLRVNSCEFP | |
| QYTTMSPVGEYPFESTEASLSSTLTPVGNVPQRIKAHLVEGKSNSKDDLRIEADASLVFEASDAQRTRR | |
| RMSLYKL | |
| S. pombe SSP1 (gi\|9075860) | |
| MGSVNNEEKTLIEPQRLLRKNTWHPEVDDSEVPPSVFPEYPVHKAIQKTSDSFRKRNYSAGDYVIAPLG | (SEQ ID NO. 28) |
| GEREGSSLTHSWTFQPGKHNQRLYSDNFQEAQRQWKRLQEWGEVKETKKIRKRFDRFSGRKYINHYEII | |
| KELGRGMHGKVKLGRDTVTRELLAIKIIPKTERRPKLGRANASSQKEKVRREIAILKKCVHPNVVRLRE | |
| VIDDPSSTKVYLVLEYHSGGEVPWTDCDSPVLSISEARQYFRDVVLGLEYLHYQGIIHRDIKPANLLLN | |
| SSNCVKISDFGVSYIANAGLNEDIWVELAKTVGTPAFFAPELCWTDLDRPRPKISEAIDVWALGVTLFC | |
| LLFGRCPFNASMEYELFDKIVNERLNIPSTPDIGEEGRDLLKRLLCKDPEQRITLVEVKLHPWTLDGLK | |
| DPEKWLQNTDPSTVSRVEVSTDEVASAISLVGRLRRKLGKLFRFRRPKARVFDSSSSVPSDSSICRPES | |
| SGNSSIGLSASELSDSFNRLAVNESQKDRERKQVHPVEMGRNSSEKKPRCDFGWDYEAFPNDNQDADDA | |
| CSYNTGDSIPQVSKSINGHFETYSRTSMDTDDVASFESPNAKHEESGMPVVTFRNYENYDANPSNFHPV | |
| VPGFVSSPNLHLAGGSDTPIYCIEHSFTPTN | |
| C. albicans ORF.7535 | |
| MSTSLSHQELSTEKGQSCPPIPDSNPLNPKSPALRTTSNSTILNSPIETINVNTSSKSNISGESTINGS | (SEQ ID NO. 29) |
| AYSNSTTVVQPEVFGEAHTTTSTHNSASTRTERNEFPNSHLHQHQQESRNNGESNTPMTSPKHFPTDDL | |
| RHSLFYKAGSAAHHKSATSSKQSSTTSLKDGLNNANTYHFQNTFLDNNVMSDLEESPVPNERNPIQDTG | |
| LGPRSHATKFGVQSTTSLPTTISQSRLPNSNKSSFFPFKSYTSSPVKETKHVFLEYDPITRRKVLNTYE | |
| ILREIGKGEHGKVKLARDLINNELVAIKIVNRKSRKERPSLRMRKNSSAPVINEYELKVKREIAIMKKC | |
| RHKHIVALREVLDDLNSLKIYLVLEYMEKGEIKWKKLQSDVAKPTANKCYDANDNEIPCCGNGRMQQRQ | |
| QSLLTDEDLLSNEFSPNLTFKQSRKIFRDVLLGLEYLHMQGIVHRDIKPANLLVSADNIVKISDFGVSF | |
| ATSLAENDEGYLVNELDLAKTAGTPAFFAPELCQFDDETATEKLSSSTESHAPPKIDYKIDIWALGVTL | |
| YCLLFGKVPFNADTEYDLFQVIVKEPLKFPNSIKAFNPPATVTEEEFELAKDLLSKMLDKNNRTRIEIQ | |
| DIKEHPFTLMDLDNDVDGLHELFHLNGDNPVEPLSFDLDEHDIVSKDEVDNAVIGVGARIKRSLVRAIR | |
| AGGLKDGEIRNKFAALQLEHSRSENSEESSSGYSNYSSSTRLLGYQNGQNYSMILSEGLPVSSATPPPA | |
| LLAAQQKRSSLLSPKSGGISEKNNPHFPSSLAHQIPNTSSPSTCSSSTSMAFQNHFSFAGMRESGKSLL | |
| HDMIESNSNNSSRRGSSAGITVSEAPQIETKRNVGGDLYLKNQSVVETFKGIQLQDDKRRRSSIFSLHS | |
| QIGTNSNKSSLSHELTPTQTGSGASTQQQHQHYSTNYSNIAAPIPVPAPRKQSTSDNEQDIKAPLLHQE | |
| KNVMGKPYLKIGPISIAREDEKSADDHPDSSIISLPLSESFASLDSINDDYLSRKYEEYTNNRKENSKS | |
| EGNVPVISLRRKSSLSESDLTRHVQLKDPFGKFKPDGSEIAEKFKAFNLGNLMKTGGKFALAEHNSSDS | |
| QQIKGNNYQSSDDGIAPKAIVPAISYSSSDSYSSCSSSSFDDEDESDDDEENLTLAFQSKVAPISRANF | |
| LSLTGRAKSHDSNLPTLRQNREKGRQLNPEFQGPIIFHDGLPEFEDVPDGLINSNPIGNNNVNGNSAFN | |
| SGYSYYDNVNPTVVSSNVSTATLTMGMAPSESVETNVEAPAQENAVKVSSPLNPHKNTSSRSDILKDLK | |
| KNTSISFGEILFNDQFNNHYKKDPVYSPFPSAKHLDNDQETIVKESASKFHEHRPTYYRSNSVTIGLLH | |
| RSTHREDDDDVSQTGNDLEQLITKEKQG | |

HAL Family

The HAL kinase family includes 7 genes from S. cerevisiae HAL5 (SEQ ID NO. 30), KKQ8 (SEQ ID NO. 31), NPR1 (SEQ ID NO. 32), PRR2 (SEQ ID NO. 33), PTK1 (SEQ ID NO. 34), PTK2 (SEQ ID NO. 35), SAT4 (SEQ ID NO. 36), YDL025C (SEQ ID NO. 37) and YOR267C (SEQ ID NO. 38). Since many are involved in transporting ions, protons and small molecules, they are intrinsically responsible for generating responses to environmental stress, e.g., to salt, pH, and osmosis, toxins and drug resistance. For instance, HAL5 (SEQ ID NO. 30) and SAT4 kinases are involved reponding to changes in salt and pH, in conjunction with activated potassium transporters. The PTK1 (SEQ ID NO. 34) and PTK2 (SEQ ID NO. 35) HAL kinases are used in polyamine transport, in part by regulation of proton pumps. YOR267C (SEQ ID NO. 38) regulates the PMA1 proton pump. PRR2 (SEQ ID NO. 33) may be involved in the pheromone mating response. Other, non-S. cerevisiae HAL family members include the SPAC29A4 (SEQ ID NO. 39) and SPCC1020 (SEQ ID NO. 40) of S. pombe and gi|3850140 (SEQ ID NO.41) and ORFx1 (SEQ ID NO. 42) isolated from C. albicans.

The bold, underlined portion of each family member denotes its kinase domain. Accordingly, the present invention allows the skilled artisan to identify and classify newly-identified kinases from other fungi as belonging to the HAL family if the newly-identified kinase domains share at least 30% amino acid sequence identity with any one of the kinase domains of an indicated family member listed below. Preferably, the HAL kinase domain has a percentage sequence identity to a kinase domain of any one of SEQ ID NOs. 30-42, of 60%, of 65%, of 70%, of 75%, of 80%, of 85%, of 90%, or of 95%.

| | |
|---|---|
| S. cerevisiae HAL5 | |
| MGDEKLSRHTSLKRARSLSESIKGLFKPSGISGSNNAAAPSSRPGQDQAHSHQTARIITSNVSSPSISP | (SEQ ID NO. 30) |
| VHSPVLQAAPKHHKLGVPNIAKLSLSPSREPSLNSENEMFSQESFISEKDEDEANLLEREDLQNKKEEK | |
| ARAKHVRSKEAYVPHHRYTVGSDEVERQPRERLKNFPQNAGSSNPANSNANHVLDQENNFSIDAMLDYD | |
| EESKLRRRNSLGVRNHSNRTRSRKNSLSTPRSPPMKNGNGGMNSNATNNVGNGTGNRIYMRGRNHSDSI | |
| SASSLPKFQEIECKCILDLGHFKVFENGYHEHSLRVLPIITNNKNVDSGDEKDADASVNSGDDGDNDSE | |
| ANMHKQKSVFSLSGLFKSHKDGNQQQQQQQQQEENGEQINLEKAFSIIPSQRFIKSQTLKKSRTSNLKN | |
| GNNDELMKNDGKNIPQIVNPNAAVGVEELKLINALSEKIRKGLKSENTKGNNGEGRSNSNKQEDSDDTE | |
| GKAGTTNDDTSHKPCSQKYGKSIGVVGAGAYGVVKICARCKTAKDVLPYSTYSNGKKLFFAVKELKPKP | |
| GDQIDKFCTRLTSEFIIGHSLSHPHFEANAMIAGNVSRTTPPK1WFNAPNILKILDLMEYSNSFVEVME | |
| FCASGDLYSLLTRNNISNESNNGSSRLIQTVKEGSGSPLHPLEADCFMKQLLNGVQYNHDHGIAHCDLK | |
| PENILFQPNGLLKICDFGTSSVFQTAWEKHVHFQSGAMGSEPYVAPEEFIRDAEYDPRLVDCWSCGIVY | |
| CTMVHGQYLWKIAIPEKDSLFKSFLSEIKDDGQFYLFEELRHVSSELNRLRKIALYRTFQVDPTKRITI | |
| EQLLQSSWMRKTKCCVVYRHLHTKVSK | |
| S. cerevisiae KKQ8 | |
| MMDSKTLTASMVMQEEKKRQQPVTRRVRSFSESFKMLFRPPRSRDSSPINVTRIPYRSSSTSPKRSSEP | (SEQ ID NO. 31) |
| PRRSTVSAQILDPKNSPIRQRSYTLKCCTPGLSHPFRQTGSGASNSPTRHRSISGEEQEIVNSLPEYKR | |
| SASHTFHGIRRPRSRSSSVSSCDSSNGTTSSSDSQWANDSLLDDSDNDLTPYRGSNKDILKSKDRAPYN | |
| YIDDYNKKALRRATSYPNPLPSKQFYNERLYTRRSHPDEESLESLPRFAGADVQCIIEQNGFKVYEDGS | |
| HEHNIKLSGVIAKLEKGNSLPAHRQGSLSRPRLGITLSGLFKHHKNECDIENALSLLPNVEKSQTNHEK | |
| RTGQSPNDSNRSSPTQGREDYLKIVNPDASLGSDELKLINSLSSRIHKSLQNYLQEKNLKPAECIGEQA | |
| PTFQDNYGHPVGLVGAGAYGEVKLCARLRNEKDSPPFETYHDSKYIYYAVKELKPKPDSDLEKFCTKIT | |
| SEFIIGHSLSHYHKNGKKPAPNILNVFDILEDSSSFIEVMEFCPAGDLYGMLVGKSKLKGRLHPLEADC | |
| FMKQLLHGVKFMHDHGIAHCDLKPENILFYPHGLLKICDFGTSSVFQTAWERRVHAQKGIIGSEPYVAP | |
| EEFVDGEYYDPRLIDCWSCGVVYITMILGHYLWKVASREKDMSYDEFYKEMQRKNQFRVFEELKHVNSE | |
| LATNRKIALYRIFQWEPRKRISVGKLLDMQWMKSTNCCLIYDST | |
| S. cerevisiae NPR1 | |
| MSSLTRLLQEKRKNETSNSSPRTSADTLTTTPESQSLDLHSRNKSSSHIGSVSNSSSSDRNRANVPVPG | (SEQ ID NO. 32) |
| SVTTVTQIYSEEDSSSTAGSSLDDRNQFSSSFLNANFAHTASFYGTSAQSRDRFGSLINDQGTAGLSSH | |
| GGSFAAQNRITSRLSTTSHTSGRAIPSLSSSIPYSVPNSNKDNNSSNSNSSSLSSSWLETYAGGMPNNI | |
| THESNVISSPKVDSVEPRFVISKQKLQKASMDSNNANATQSRSISRSGSFSSQLGNFFFSKNSKESSNS | |
| NSAGMSFSANSNGPSPNIKNPNVTNGSTPIPKPIRARQSSIYSASRQPTGSYTDNFYGSPSSVHDHLPP | |
| SQSVPRSQHSSIGDLKRFFKKSSNSNLSSNSNNVIPNGSPLSSGIAVPSHSHSSSHFAAGNNSYSTSYN | |
| GNGDTIYSHSHGGSGIPFSKRYIKTGADLGAGAGGSVKLAQRISDNKIFAVKEFRTKFENESKRDYVKK | |
| ITSEYCIGTTLNHPNIIETIEIVYEIWRILQVMEYCEYDLFAIVMSIKMSYEEICCCFKQILTGVQYLH | |
| SIGLAHRDLKLDNCVINEKGIVKLIDFGAAVVFSYPFSKNLVEASGIVGSDPYLAPEVCIFAKYDPRPV | |
| DIWSSAIIFACMILKKFPWKIPKLRDNSFKLFCSGRDCDSLSSLVTRTPDPPSYDESHSTEKKKPESSS | |
| NNVSDPNNVNIGPQRLLHSLPEETQHIVGRMIDLAPACRGNIEEIMEDPWIRSIDMCHLVEDGLSFKVV | |
| RGEDHHHTQVDQSEAHIAGLEKKKKKQNNQ | |
| S. cerevisiae PRR2 | |
| MSLSRILRYNQRNNKTTASLTAEHAYSDNWAYSVSLGDPTSVGVNMAAKTGEALNKSYDSVFSSLPVAD | (SEQ ID NO. 33) |
| SVPRTDFTASSRDDENTDVQKLTTSWMEKIDTKMPENISKIDSNIISSPMVSKVEARFIVPKGRLRKNS | |
| TDFTSSFSNSLSLPKSYGKLIFFTSKKNSSSTKKNLANDISDNKHNNNSSNTIGHNIPVTTATATCDEI | |
| ACTSTEHEYNVYEEERMFTTRVYSLEDSVSSLSTNPLDDTYSEAVQVNTRHIEDTESTAHIRKHSYTTS | |
| LSSIKRLFKITSFSNNNSNSCDHQESTVADDCAISSSLKETTSSPVSTGSFSLMIENEDSDRDQIIQAL | |
| YSNIEASTDLVSRKYRDLDVVLGEGSGGKVKLVQRVLDNKVFALKEYRSKKKRESERKYIKNIISEYCI | |
| ASTLKNPNICETLEILYEKGKIFQILEYCEYDLFSLVMSEKMKYEEICCLFKQLINGVKYLHIGLSHR | |
| DLKLDNCVVTRRGILKLIDFGASSVFHYPLSSQMIEANGIVGSDPYLSPEVFYFNEYDPRALDVWSVGI | |
| IFFCHITRRFPWKYPKVKDVQFKAFCSGRGVSSFKDLVTRPATDDSNNYDNDGYEEGVIDMGPNFILHR | |
| LPEETHKIMRRILEVSPFRRITINGILQDGWIKEIETCQVVGAASPNEASLRIINKGNHIHTNIDQRYA | |
| HIGGLHQRT | |
| S. cerevisiae PTK1 | |
| MTVSHNHSTKISQQPISSVSAFKFFGKKLLSSSHGNKLKKKASLPPDFHSTSTNDSESSSPKLPNSLKT | (SEQ ID NO. 34) |
| SRRANSFAHTTMSKRSLSSASTKILPPAGSSTSISRGNRHSSTSRNLSNSKFSSERLVYNPYGVSTPST | |
| SLSSVSTSMKKDPDLGFYLHDGDSKIRMLPIPIVDPNEYLPDEMKEASIQLSDNFVFDDENKTIGWGGS | |
| CEVRKIRSKYRKKDVFALKKLNMIYNETPEKFYNAAPKEFIIAKQLSHHVHITNTFLLVKVPTTVYTTR | |
| GWGFVMELGLRDLFAMIQKSGWRHVALAEKFCIFKQVACGVKFCHDQGIAHRDLKPENVLLSPDGVCKL | |
| TDFGISDWYHHGSTRPVQPCQEVRRDDRLAPYAPPEVMFYDSKKRYDTELQQPYDPRALDCYGLGIILM | |
| TLVNNVIPFLESCSFDTGFRDYCDAYENFIRLHDRAFRNRAITARGREWSITWLEISRTDMHLAWHGGS | |
| LTQKPPPATPSTTSSKTHGSKELRLVLMPTTNMCVRNLLSKPLRTRIRGVSISLQMLLQPHPPQTHSLR | |
| TESPSGQW | |
| S. cerevisiae PTK2 | |
| MAGNGKDKEVDKSPSVSTLKLLGKRLFNSSSHTDNSSLLLSAEQLGNGRSLRKRPTSPSISGSGSGGNS | (SEQ ID NO. 35) |
| PSSSAGARQRSASLHRRKNNASVGFSNGSVSSHKSSVALQDLIKHNNNPYLNSPSDILGTGTGIASTRD | |
| RDRAVLDREKEKERARNKERNTHHAGLPQRSNSMASHHFPNENIVYNPYGISPNHARPDTAFADTLNTN | |
| KENDLSFYMHDGNSKIRMLPLPIANPNDFLPEDMKQYSVHLTDNFVFDTDNKPIGSGGSSEVRKVKSSY | |
| RQKDVYALKKLNMIYHESPEKFYKRCSKEFIIAKHLSHNVHITFEFYLLKVPTTTYTTRGWGFIMELGV | |
| KDLFQLMERTGWKNVPFNEKYCLFKQVAQGIKFCHDNGIAHRDLKPENVLISKEGICKLTDFGISDNYH | |
| VIPHDYTSPVKTCQGMIGSPPYTPPEVMYFDAKKHYPEKFQKPYNPLAMDSYALGIMLITMINNIIPFI | |
| DSCNTDARFREFEVSYDNFINHQNPHFRDKGCRKPGPGSEYSLARNFKNTDATRIAWRLADPNPATRYT | |
| MDDLFNDPFFQQIETCVEPNDDDLVRVPELRKSTSTNDFSENSLDAPHDQEVIHTSNPFLKKETLTSKP | |
| RSMLEIAESPSLKQKSKVKDSAKTKTHDVGDEGGNESTKPKQQDKKENLKKDEVKNGDKDKVIEEATTT | |
| NVDSILEKPTPTSTKVEDNLSEDDSTMKELKSMLNSTPTTPTHNGPTPLPAKAGTQLDKRMSDLSLKSE | |
| TPASTKNFSAPNVSSSSNSLRSLGSPSVSSSKKKKVIHHHLDITNSVTNMSSVSAFISR | |
| S. cerevisiae SAT4 | |
| MTGMNDNNAAIPQQTPRKHALSSKVMQLFRSGSRSSRQGKASSNIQPPSNINTNVPSASKSAKFGLHTP | (SEQ ID NO. 36) |
| TTATPRVVSNPSNTAGVSKPGMYMPEYYQSASPSHSSSSASLNNHIDINTSKSSSAASLTSSVSALSLS | |
| PTSAINISSKSLSPKFSHHSNSNTAITPAPTPTASNINNVNKITNTSAPICGRFLVHKDGTHEHHLKNA | |
| KRQEKLSTMIKNMVGASKLRGEAKSAVPDIIMDPKTTLKSNKNPPTLFAGFMKQVVDMDDKYPEGAPTS | |
| GALNCPERDIYRSDQKDSKNNTHNITTTKKDRQCFAEKYGRCQEVLGKGAFGVVRICQKKNVSSQDGNK | |
| SEKLYAVKEFKRRTSESAEKYSKRLTSEFCISSSLHHTNIVTTLDLFQDAKGEYCEVMEYCAGGDLFTL | |
| VVAAGKLEYMEADCFFKQLIRGVVYNHEMGVCHRDLKPENLLLTHDGVLKITDFGNSECFKHAWEKNIH | |
| LSGGVCGSSPYIAPEEYIKEEFDPRPVDIWACGVIYMAMRTGRQLWSSAEKDDPFYMNYLKGRKEKGGY | |
| EPIESLKRARCRNVIYSMLDPVPYRRINGKQILNSEWGREIKCCHNGRALK | |
| S. cerevisiae YDL025C | |
| MVKETFLHSSSSTSLSSLFRPTKLKMLSAKIFNGGGNQSYSKTDDVSRSSSRSSKKNTDSDQEDQIKYN | (SEQ ID NO. 37) |
| KPNDRRSTIGKSPQGNGALSKESHVVASSTLTGISPTSAKKAPIDYSPSRPLPNNHNPVRTGHTVPHLP | |
| HSIHNPINYIHQGSKDAFHHPHPVRSTAHSNKSTVSSAKSDTPSSNLSYQAHMHPVEILQKQIEDKHFM | |
| DSQASTPGSVELQHNSSSGSDDTSSRKKKSLRLTRFFKKIHNDYHDNHHHHHHHNRGSTPTKPKLNLNT | |
| NENIVESNGKALYETDNPVELLEKYGIPGRKLGEGASGSVSVVERTDGKLFACKMFRKPHLNNEGTNQS | |
| QLANYSKKVTTEPCIGSTLHHENIVETLDHLTEGDTYLLVMEYAPYDFFNLVMSNLMTQDEVNCYFKQL | |
| CHGVNYLHSMGLAHRDLKLDNCVVTKDGILKLIDFGSAVVFQYPYEDTIVKSHGIVGSDPYLAPELLKQ | |
| TSYDPRVADVWSIAIIFYCMVLKRFPWKAPKKSFNSFRLFTEEPEDEDDIVRGPNKILRLLPRHSRTII | |
| GRMLALEPKQRVLWQDVVKDDWLVSVPSCEVDPTSGDLVEKPKNHKHHLVTEEELNELTKQHGNKDSN | |
| S. cerevisiae YOR267C | |
| MPNLLSRNPFHGHHNDHHHDRENSSNNPPQLIRSSKSFLNFIGRKQSNDSLRSEKSTDSMKSTTTTTNY | (SEQ ID NO. 38) |
| TTTNLNNNTHSHSNATSISTNNYNNNYETNHHHNISHGLHDYTSPASPKQTHSMAELKRFFRPSVNKKL | |
| SMSQLRSKKHSTHSPPPSKSTSTVNLNNHYRAQHPHGFTDHYAHTQSAIPPSTDSILSLSNNINIYHDD | |
| CILAQKYGKLGKLLGSGAGGSVKVLVRPTDGATFAVKEFRPRKPNESVKEYAKKCTAEFCIGSTLHHPN | |
| VIETVDVFSDSKQNKYYEVMEYCPIDFFAVVMTGKMSRGEINCCLKQLTEGVKYLHSHGLAHRDLKLDN | |
| CVMTSQGILKLIDFGSAVVFRYPFEDGVTHAHGIVGSDPYLAPEVITSTKSYDPQCVDIWSIGIIYCCM | |
| VLKRFPWKAPRDSDDNFRLYCMPDDIEHDYVESARRHEELLKERKEKRQRFLNHSDCSAINQQQPABES | |
| NLKTVQNQVPNTPASIQGKSDNKPDIVEEETEENKEDDSNNDKESTPDNDKESTIDIKISKNENKSTVV | |
| SANPKKVDADADADCDANGDSNGRVDCKANSDCNDKTDCNANNDCSNESDCNAKVDTNVNTAANANPDM | |
| VPQNNPQQQQQQQQQQQQQQQQQQQQHHHHQHQNQDKAHSIASDNKSSQQHRGPHHKKIIHGPYRLLRL | |
| LPHASRPIMSRILQVDPKKRATLDDIFNDEWFAAIAACTMDSKNKVIRAPGHHHTLVREENAHLETYKV | |
| S. pombe SPAC29A4 (gi\|2239225, ortholog of SAT4) | |
| MGEKDKLHEISSKFASLGLGSLKSTPKARETTEPPPPSSQQPPSTPNGKEAASPSALKQNVRPSLNSVQ | (SEQ ID NO. 39) |
| QTPASIDAVASSSNVSLQSQQPLSKPVVSSKPNQTTAMPPPSNNPSRHVSSTSNKPAAVSPNPAAHHAE | |
| LPSGSVPPSASVSRANSTATTTPHKAGVVSNPAAANVHVLSVAASPNPSTPSNGPAPVSTTATPSRNPV | |
| TRLQRIFSQNSVSRQNSRTGRGAAVANTEETNSTGGSETGGAANSSSTSNPSSAKWSRFTVYDDASHTH | |
| QLRPARRQEKLGKMLKDFLAGNSKKREEERIAKEAADAQHQLSLVQSWINGYGQEKLADKKDPAKVSAS | |
| FVEKYGRCQEVIGRGAFGVVRIAHKVDPQNSGSETLYAVKEFRRKPAESQKKYTKRLTSEFCISSSLRH | |
| PNVIHTLDLIQDGKGDYCEVMELCSGGDLYTLIMAAGRLEPMEADCFFKQLMRGVDYLHDMGVAHRDLK | |
| PENLLLTVSGSLKITDFGNGECFRMAWEKEAHMTCGLCGSAPYIAPEEYTESEFDPRAVDVWACGVIYM | |
| AMRTGRRLWRVAKKSEDEYYSRYLHDRKNESGYEPIEMLERSRCRNTLYNILHPNPTYRLTAKQIMKSE | |
| WVRSITLCEAGNAGL | |
| S. pombe SPCC1020 (gi\|3130053) | |
| MSVTPPNVQFNLNGDSDHKSDNSSSSLENKLDTELKITSPPRNPPQRLHPVDFSEHADTDDDMNHPLPR | (SEQ ID NO. 40) |
| VQSPVHIKNHIDPKLAEDRYRSSAARHFEPISIPPSAITSEDEDDYHGSANSSTVLPPRTENALHAASP | |
| KPSGSTGYTSPALSQNSGSGGEGESDEGSFNTQHHRSPIFQAYPSSEDLVGDPNDPYRRTRRAPIKTNP | |
| HDIPSQFIFRKLGLHHGKHGHHGHSGSLSLKSLVPNHHDKHDKHDKHEKMHSSLDLRRFFKSHQKTDKE | |
| KKPSVSKSKSSANLQDDHFGLFKKYGKFGRMLGSGAGGSVRIMKRSSDGKIFAVKEFRARRPTETEREY | |
| ARKVTAEFCIGSALHHTNIIETLDIVEENKKFYEVMEYAPYDMFSIVMSGKMTMPEVYCCFKQLLSGVA | |
| YLHSMGLAHRDLKLDNLVVDSNCFVKIIDFGSAVVFKYPFEADIVEATGVVGSDPYLAPETLVRKLYDP | |
| RAVDIWSSAIIFCCHALRRFPWKYPKLSDNSFRLFCMKQPSNNAESPSDILADIKKQRLVEQGCEPIRK | |
| TDESHSPNSKTDNSSTHKQELYGPWRLLRLLPRETRAVIAHMLELDPVKRYDIHRVFAINWINDISMCH | |
| MENGKVIHSPTHVHNLVASEESPAPPAKH | |
| C. albicans (gi\|3850140) | |
| MTKEHSIRNIFKKDKTPDNGSATATPSSSHTGLSKLFHKESKPITPPMKRTPSVSSLKRRNTNPSQTSG | (SEQ ID NO. 41) |
| ISLNHNHHHHQDSQNHNDATTSGGNIHSSTPVNRSRSNSDRLGHVPPTGRKVLSKAETFTHLQQLDTRN | |
| AAKNQLRNHRIPSNHLSSPLSAAPHSDKIVYNPYGLNKTATQERPKNTSFYLSGVNDGERVLSNPVASP | |
| NDYLPAELQQQHVNLLEDFEIDVGTKKLGDGGSSDVRIINSCHHKKDLYALKKFTLLSKETDEDFYKRV | |
| SEEYKIHRKAAISRHVVDAFAILRIQSQSNLTRGWGMVMEFCGGGDLFSVIVKPGWKSTPLAEKYCLFK | |
| QIAYGVKFLHDHDIVHRDLKPENVLLDANGLAKLCDFGVSEFGHEVPEDFSSPVKLSTAYVGSPPYAPP | |
| EVMLLKEKSSTEIKAFAYDPFKMDCWGLGMLLFCLVYGGVPFQQSSPNDHAFRDYKFSHKRFCTDHATF | |
| KSNQGYPRGPGSEFKLAAKFENNGASRVAWKLCDPSENTRYTMDMLFDDPWFQSVEMCIYESPDQEVNP | |
| FVLPGTGENIDTHSVSGYSSVNNSQAPSRRGTFTSRPVGSGAGSGYNSHDESSNGLSSSFRSMLDLKDI | |
| PQKITKTDEPLPSNSSVHSNDSSSAPAKSKLDHPSSPGSLLSPSTPALQSIPADRVAQTTSPINASLPA | |
| VEESDIEHESESEIQGDETESSGLQVLPPIDDVVAASPSSLTHEPQEQVLDSVESCISLPPNRDQAFAG | |
| KDGEMCSLVDLKPAALKSATDLQLGADGMCNLGYKIKKHHHTEVSNVSNSSRR | |
| C. albicans ORFx1 | |
| MSSLTKLLHESTSTLASPVLSRNTSEVTFKDQGRRTPEILNISDTVDAKSPGITIDVSKPKPSPIDTDG | (SEQ ID NO. 42) |
| MNVEPQAVHGFDPSPNTKVSFSSPFSPTSPFTRQSSNSFSSNQAFQNTRGAVASPRYIKNNSVSHSSVF | |
| MGGESLSSSIPYSAPGGGRGNPASHSGNTGSLQRENSFSSLNTSDSNSSAHIPNLPNGQPINSIHIQSP | |
| QVSASSIDSRFVVSKQRIAQAQAQASLSSSQRS&SQSGLSFFFSQKSKPAVKRDSTTDLGAFYNNSYQD | |
| RDAPIVSGSPNSLSSAESTVSYGSSAPTRHNSMANLKRFFKKSTPTTSQPVGTSNLSSSLRSASSGASG | |
| ANNIPNSLNGQTNNGYQSPSSFSASTSNTSYSQSPGTNSSSVTRSSTLQNKVNYHERRQSVSGIVNNSQ | |
| QLPFSKRYHSKNAESLGAGAGGSVRLLTRVSDGLTFAVKEFRAKYQNESKRDYAKKITGEYCIGSTLKH | |
| PNIIETVEICYENERIHQVMEYCDFDLFAIVMSNKMSREEINCCFKQILAGVHYLHSMGLAHRDLKLDN | |
| CVIDKRGIVKIIDFGSAVVFSYPFTKTLIEAQGIVGSDPYLAPEVCVFNKYDPRPVDVWSVAIIYCCMM | |
| LKKFPWKVPKLSDSSFKLFASRGEFIPISEHLKKTPNDMEKSNSNGSSGGGLSNLEDISEALEDEITAG | |
| AKQKPSTTGQNGATANGKDHTSSETGANRLLLALPEDCRRLIGRMVELAPACRITIDEVLNDSWLKSVN | |
| MCTVEESSPGVFEVIKCEDHEHTQVDQSKAHIAAFEKNKKK | |

DNA encoding any one of these kinase domains or full-length sequences can be cloned into an expression vector and expressed in a suitable expression system. The resultant kinase protein can then be purified, tested for kinase activity and then exposed to test compounds to determine if that activity changes, in particular if the kinase activity reduced. Similarly, a gene encoding any fungal kinase that has the indicated sequence identity across its kinase domain, with one of the above-described fungal kinase family members, may be isolated, expressed and also used to determine which test compounds modulate kinase activity. Such compounds can be used to inhibit fungi and can be used to treat fungal infections.

A fungus that contains a kinase having a sequence of any one of SEQ ID NOs. 1-42, or that contains a kinase that has a minimum sequence identity across its kinase domain, may be exposed to the test compound(s) to determine if inhibiting kinase activity has a biological consequence, such as death or retardation of growth of the fungus. Accordingly, a test compound used to target a fungus containing a kinase classified to one of these families, should not have any effect, i.e., a modulatory, inhibitory, or deleterious effect upon kinase activity in a non-fungal organism.

In general, determining kinase activity is a well known method in the field to which the art of this invention pertains. Indeed, there are many kits, reagents and products available commercially that perform this type of assay. In general, it is possible to determine the extent of kinase activity by determining the rate, level or ability of the kinase to phosphorylate a substrate protein. Determining phosphorylation levels and patterns of a kinase is well within the capability of the skilled artisan. Such a test can be performed on an isolated or purified kinase or from a preparation of lysed fungal cells. A control kinase, such as that from a non-fungal organism, can be tested alongside the target fungal kinase. Ideally, the phosphorylation pattern and levels of the non-fungal kinase substrate is unchanged after exposure to a test compound, while the fungal form is altered to reflect reduced or even abolished kinase activity. The art is also replete with techniques for determining the activity of a variety of kinases. For instance, there exist assays for determining Src-family protein tyrosine kinase activity by measuring the consumption of the exogenous substrate, enolase; there exist protein kinase C assays, histone kinase assays, and in vitro kinase assay protocols. An in vivo kinase activity kit is available commercially, through Clontech to determine the activity of kinases in signal transduction pathways. A compilation of kinase assay protocols is available as of Jun. 7, 2002, from the website, http://bric.postech.ac.kr/protocols/general_methology/general_protocol_{—}42 .html.

Very generally, the amount of a radioactively-labeled ATP molecule, e.g. [gamma-32P] ATP, that is recorded by a scintillation counter, for example, after a kinase reaction with substrate is used to indicate the activity of the kinase. The specific activity is typically related in cpm/pmol and when assaying a purified kinase, the catalytic rate is best expressed during its linear range in mol phosphate transferred from ATP to substrate/min/mg of kinase. Highly active kinases transfer on the order of micromol phosphate/min/mg of kinase. Indeed, U.S. Pat. No. 6,399,319, describes a protein kinase assay for identification of fungicides, by exposing the kinase to a phosphate donor ([gamma-33 P]ATP) and substrate. The amount of radioactive phosphate measured after the reaction is terminated is an indicator of kinase activity with and without the presence of a potential candidate fungicide.

There exist, therefore, several established assays that one may employ to determine the extent, if any, to which a compound has antifungal properties. For example, one may screen compounds for their ability to eradicate or slow down the growth of a fungus in culture, by staining the fungus or measuring the density, or concentration, of spore formation after a period of incubation with a test compound.

Accordingly, a method of identifying a compound having antifungal properties, may comprise (a) culturing a fungus sample that contains at least one of SEQ ID NOs. 1-42; (b) treating the fungus sample with a test compound; and then (c) determining the level of activity of the fungus in comparison to an untreated control fungus sample. An antifungal agent would be one that brings about a decrease in the level of fungus activity (measured by activity, viability, growth status or amount of fungus) of the treated fungus.

Chitin-Stain Antifungal Assay

Chitin is typically present in fungal hyphae and, therefore, is a good indicator of the abundance or growth of a fungus. Thus, staining for chitin levels is a quick and convenient method for determining fungal growth in the presence of a test compound, i.e., a potential fungicide.

Accordingly, one may use a iodine-potassium iodide solution (2 gm KI in 100 ml water and add 0.2 gm 12) in 1% sulfuric acid to stain for the presence of chitin in a cultured fungal sample before and after exposure to the test compound. Typically, a high concentration of chitosans stain dark violet, whereas lower amounts stain light blue. With this particular assay, however, cellulose may also be stained. For this reason, if necessary, cellulose can be removed by flooding with Schweitzer's reagent (saturated copper hydroxide in ammonium hydroxide) prior to iodine-potassium iodide treatment.

Agar Dilution Antifungal Assay

Another assay for antifungal activity is described in U.S. Pat. No. 5,837,726. In that document antifungal properties were determined by an agar dilution assay on microtiter plates. This assay monitors the growth of fungal spores in the presence of a test substance. In short, a spore preparation of a fungus is prepared by streaking out spores from a fungal stock suspension onto an agar plate for germination. After incubating the spores for 37° C. for a period of time, the spores are washed, counted and stored at 40° C. From this preparation, a spore suspension of known concentration can be made.

The next step involves aliquoting serial dilutions of a test substance into a microtiter plate to which agar is added and allowed to solidify. The spore suspension is then also aliquoted into each well and the plate incubated at 35° C. for 48 hours. The concentration of spore germination in the microtiter plate is determined by taking OD₆₅₀ readings in a microtiter plate reader. According to the methodology of the '726 patent, at OD₆₅₀, a value of “0” reflects 100% growth inhibition (or 0% growth); a value of 1 corresponds to 75% growth inhibition; a value of 2 corresponds to 50% growth inhibition; a value of 3 corresponds to 25% growth inhibition; and a value of 4 corresponds to no growth inhibition.

What is claimed is:

1. A method for identifying a compound that inhibits the activity of at least one of KIN1 kinase, KIN4 kinase, GIN4 kinase, RAN kinase, ELM kinase, or HAL kinase in a fungus comprising determining the activity of the kinase before and after exposing the fungus to a test compound, wherein a reduction in kinase activity in the presence of the test compound indicates that the test compound is an antifungal agent, wherein the test compound has minimal toxicity to a non-fungal organism, and wherein the kinase domain of the KIN1 kinase has at least 46% sequence identity to a kinase domain of any one of SEQ ID NOs. 1-5, wherein the kinase domain of the KIN4 kinase has at least 55% sequence identity to a kinase domain of any one of SEQ ID NOs. 6-9, wherein the kinase domain of the GIN4 kinase has at least 55% sequence identity to a kinase domain of any one of SEQ ID NOs. 10-16, wherein the kinase domain of the RAN kinase has at least 55% sequence identity to a kinase domain of any one of SEQ ID NOs. 17-24, wherein the kinase domain of the ELM kinase has at least 38% sequence identity to a kinase domain of any one of SEQ ID NOs. 25-29, and wherein the kinase domain of the HAL kinase has at least 30% sequence identity to a kinase domain of any one of SEQ ID NOs. 30-42.

2. The method of claim 1, wherein the test compound reduces fungal growth.

3. The method of claim 1, wherein the test compound eradicates the fungus.

4. The method of claim 1, wherein the kinase activity is determined by comparing protein phosphorylation patterns in the fungus in the presence and absence of the test compound.

5. The method of claim 1, wherein the non-fungal organism is a mammal, animal, tree, or plant.

6. The method of claim 5, wherein the mammal is a goat, sheep, cattle, horse, cat, dog, pig, rat, mouse, primate, pig, or a human.

7. The method of claim 1, wherein the non-fungal organism is a fish, bird, or a reptile.

8. The method of claim 5, wherein the plant is selected from the group consisting of barley, wheat, corn, rice, cotton, oak, tomato, potato, Dutch elm, and Chestnut.

9. The method of claim 1, wherein the kinase domain of KIN1 kinase, KIN4 kinase, GIN4 kinase, RAN kinase, ELM kinase, and HAL kinase has between 80-90% sequence identity to a kinase domain of any one of SEQ ID NOs. 1-5, 6-9, 10-16, 17-24, 25-29, 30-42 respectively.

10. A method of identifying a compound having antifungal properties, comprising:
(a) culturing a fungus sample,
(b) treating the fungus sample with a test compound;
(c) determining, after the treating of step (b), the level of activity of the fungus the sample in comparison to an untreated control fungus sample;
wherein a decrease in the level of fungus activity of the treated fungus, compared with the control sample, indicates that the test compound is an antifungal agent, and wherein the fungus comprises at least one of a KIN1 kinase, a KIN4 kinase, a GIN4 kinase, a RAN kinase, an ELM kinase, or a HAL kinase.

11. The method of claim 10, wherein the kinase domain of the KIN1 kinase has at least 46% sequence identity to a kinase domain of any one of SEQ ID NOs. 1-5, wherein the kinase domain of the KIN4 kinase has at least 55% sequence identity to a kinase domain of any one of SEQ ID NOs. 6-9, wherein the kinase domain of the GIN4 kinase has at least 55% sequence identity to a kinase domain of any one of SEQ ID NOs. 10-16, wherein the kinase domain of the RAN kinase has at least 55% sequence identity to a kinase domain of any one of SEQ ID NOs. 17-24, wherein the kinase domain of the ELM kinase has at least 38% sequence identity to a kinase domain of any one of SEQ ID NOs. 25-29, and wherein the kinase domain of the HAL kinase has at least 30% sequence identity to a kinase domain of any one of SEQ ID NOs. 30-42.

12. The method of claim 10, wherein the step of determining fungal activity in the fungus sample comprises at least one of determining chitin content or performing an agar dilution assay.

13. The method of claim 12, wherein a decrease in chitin staining or reduction in optical density indicates a reduction in growth of the fungus in the fungus sample.

14. A method for treating a subject having a fungal infection, comprising administering to a subject a compound capable of inhibiting kinase activity in a fungus, wherein the kinase comprises an amino acid sequence selected from the group consisting of SEQ ID NOs. 1-42, and wherein the compound does not inhibit kinase activity of a kinase that is endogenous to the subject.

15. A method of claim 14, wherein the compound reduces growth of the fungus or eradicates the fungus

16. The method of claim 14, wherein the subject is a plant selected from the group consisting of barley, wheat, corn, rice, cotton, oak, Dutch elm, and Chestnut.

17. The method of claim 14, wherein the fungus is located on the skin, hide or external surface of the subject.

18. The method of claim 14, wherein the subject is a mammal.

19. The method of claim 18, wherein the mammal is a goat, sheep, cattle, horse, cat, dog, pig, rat, mouse, primate, pig, or a human.

20. The method of claim 14, wherein the subject is a fish, bird, or a reptile.

21. The method of claim 14, wherein the compound is administered to the subject by spraying, injecting, ingesting, inhaling, swallowing or applying a topical cream, gel, liquid, powder, pellet, aerosol or fluid suspension containing the compound to the fungus on the subject.

22. The method of claim 14, wherein the fungus is an Ascomycetes, Zygomycota, Deuteromycota, Mycophycophyta, Ascomycota, Gasteromycetes, Myxomycota, Oomycota or Hymenomycetes fungus.

23. The method of claim 1, 10, or 14, wherein the fungus is an Aspergillus flavus, Aspergillus fumigatus, Aspergillus glaucus group, Aspergillus nidulans, Aspergillus niger, Aspergillus terreus group, Blastomyces dermatitidis, Candida albicans, Candida tropicalis, Candida glabrata, Candida parapsilosis, Candida krusei, Candida lusitaniae, Coccidioides immitis, Histoplasma capsulatum var. capsulatum, Paracoccidioides brasiliensis, Sporothrix schenckii, Absidia, Apophysomyces, Cokeromyces, Cunninghamella, Mucor, Rhizomucor, Rhizopus, Saksenaea, Syncephalastrum, Mortierella, Basidiobolus, Conidiobolus, Trichophyton, Microsporum gallinae, Microsporum canis mycorrhiza, arbuscular mycorrhiza, vesicular-arbuscular mycorrhiza or Ectomycorrhiza.

24. A pharmaceutical composition, suitable for administration to a subject, comprising a compound that inhibits activity of a kinase in a fungus but does not inhibit any kinase endogenous to a subject infected with the fungus.

25. The pharmaceutical composition of claim 24, wherein the compound inhibits a kinase that has kinase domain amino acid sequence that has (i) at least 46% sequence identity to a kinase domain of any one of SEQ ID NOs. 1-5, or (ii) at least 55% sequence identity to a kinase domain of any one of SEQ ID NOs. 6-9, or (iii) at least 55% sequence identity to a kinase domain of any one of SEQ ID NOs. 10-16, or (iv) at least 55% sequence identity to a kinase domain of any one of SEQ ID NOs. 17-24, or (v) at least 38% sequence identity to a kinase domain of any one of SEQ ID NOs. 25-29, and or (vi) at least 30% sequence identity to a kinase domain of any one of SEQ ID NOs. 30-42.

26. The method of claim 10, wherein the fungus sample is a fungus or fungus extract.
